(19) United States
(12) Patent Application Publication
(10) Pub. No.: US 2009/0215777 A1
(43) Pub. Date: Aug. 27, 2009

(54) Pyrimidine Derivatives As HSP90 Inhibitors

(75) Inventors: Gianni Chessari, Cambridge (GB); Miles Stuart Congreve, Cambridge (GB); Owen Callaghan, Cambridge (GB); Suzanna Ruth Cowan, Cambridge (GB); Christopher William Murray, Cambridge (GB); Alison Jo-Anne Woolford, Cambridge (GB); Michael Alistair O'Brien, Hitchin (GB); Andrew James Woodhead, Cambridge (GB)

Correspondence Address: HESLIN ROTHENBERG FARLEY & MESITI PC, 5 COLUMBIA CIRCLE, ALBANY, NY, 12203, US

(73) Assignee: Astex Therapeutics Ltd., Cambridge (GB)

(21) Appl. No.: 11/914,728

(22) PCT Filed: May 19, 2006
(86) PCT No.: PCT/GB2006/001851

§ 371 Date: Jul. 8, 2008

Related U.S. Application Data
(60) Provisional application No. 60/682,687, filed on May 19, 2005.

(30) Foreign Application Priority Data
May 19, 2005 (GB) 0510252.0

(51) Int. Cl. A61K 31/5377; C07D 239/42; A61K 31/505; C07D 413/12; C07D 251/42; A61K 31/53

(52) U.S. Cl. 514/235.8; 544/332; 514/275; 544/122; 544/211; 514/245

(57) ABSTRACT

The invention provides a compound for use as an inhibitor of Hsp90, the compound having the formula (I): or salts, tautomers, solvates or N-oxides thereof; wherein: A is N or a group CR³; R¹ is a monocyclic or bicyclic carbocyclic or heterocyclic ring of 5 to 10 ring members of which up to two ring members may be heteroatoms selected from N, O and S and the remainder are carbon atoms, the carbocyclic or heterocyclic ring being optionally substituted by one or more substituent groups independently selected from R¹⁰; and R², R³ and R¹⁰ are as defined in the claims.

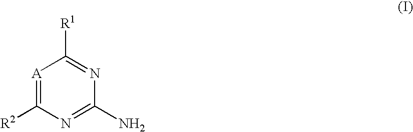

This invention relates to compounds that inhibit or modulate the activity of the heat shock protein Hsp90, to the use of the compounds in the treatment or prophylaxis of disease states or conditions mediated by Hsp90, and to novel compounds having Hsp90 inhibitory or modulating activity. Also provided are pharmaceutical compositions containing the compounds and novel chemical intermediates.

### BACKGROUND OF THE INVENTION

In response to cellular stresses including heat, toxins, radiation, infection, inflammation, and oxidants, all cells produce a common set of heat shock proteins (Hsps) (Macario & de Macario 2000). Most heat shock proteins act as molecular chaperones. Chaperones bind and stabilize proteins at intermediate stages of folding and allow proteins to fold to their functional states. Hsp90 is the most abundant cytosolic Hsp under normal conditions. There are two human isoforms of Hsp90, a major form Hsp90α and minor form Hsp90β. Hsp90 binds proteins at a late stage of folding and is distinguished from other Hsps in that most of its protein substrates are involved in signal transduction. Hsp90 has a distinct ATP binding site, including a Bergerat fold characteristic of bacterial gyrase, topoisomerases and histidine kinases. It has been shown that ATP bound at the N-terminal pocket of Hsp90 is hydrolysed. This ATPase activity results in a conformational change in Hsp90 that is required to enable conformational changes in the client protein.

A dimerization domain and a second ATP binding site, which may regulate ATPase activity, is found near the c-terminus of Hsp90. Dimerization of HSP90 appears critical for ATP hydrolysis. Activation of Hsp90 is further regulated through interactions with a variety of other chaperone proteins and can be isolated in complex with other chaperones including Hsp70, Hip, Hop, p23, and p50cdc37. Many other co-chaperone proteins have also been demonstrated to bind HSP90. A simplified model has emerged in which ATP binding to the amino terminal pocket alters Hsp90 conformation to allow association with a multichaperone complex. First the client protein is bound to an Hsp70/Hsp40 complex. This complex then associates with Hsp90 via Hop. When ADP is replaced by ATP, the conformation of Hsp90 is altered, Hop and Hsp70 are released and a different set of co-chaperones is recruited including p50cdc37 and p23. ATP hydrolysis results in the release of these co-chaperones and the client protein form the mature complex. Ansamycin antibiotics herbimycin, geldanamycin (GA) and 17-allylamino-17-desmethoxygeldanamycin (17-AAG) are ATP binding site inhibitors that block the binding of ATP and prevent conversion to the mature complex (Grenert et. al., 1997. J Biol. Chem., 272:23834-23850).

Despite Hsp90 being ubiquitously expressed, GA has a higher binding affinity for Hsp90 derived from tumour vs. normal cell lines (Kamal et. al, Nature 2003; 425: 407-410). GA also shows more potent cytotoxic activity in tumour cells and is sequestered at higher concentrations within tumours in xenograft mouse models (Brazidec J. Med. Chem. 2004, 47, 3865-3873).

There is some evidence that Hsp90 is found primarily within “activated” multichaperone complexes in the tumour cells as opposed to “latent” complexes in normal cells. One component of the multichaperone complex is the cdc37 co-chaperone. Cdc37 binds Hsp90 at the base of the ATP binding site and could affect the off rates of inhibitors bound to Hsp90 in the “activated” state (Roe et. al., Cell 116, (2004), pp. 87-98). The client protein bound to the Hsp90-Hsp70 form of the chaperone complex is believed to be more susceptible to ubiquitination and targeting to the proteasome for degradation. E3 ubiquitin ligases have been identified with chaperone interacting motifs and one of these (CHIP) was shown to promote the ubiquitination and degradation of Hsp90 client proteins (Connell et al., 2001. Xu et. al., 2002).

### Hsp90 Client Proteins

The number of reported Hsp90 client proteins now exceeds 100. Since many of its client proteins are involved in cell signalling proliferation and survival, Hsp90 has received major interest as an oncology target. Two groups of client proteins, in particular cell signalling protein kinases and transcription factors, suggest Hsp90 regulation may have potential benefit as an anticancer therapy.

Hsp90 protein kinase client proteins implicated in cell proliferation and survival include the following:

c-Src

Cellular Src (c-Src) is a receptor tyrosine kinase required for mitogenesis initiated by multiple growth factor receptors including the receptors for epidermal growth factor receptor (EGFR), platelet-derived growth factor receptor (PDGFR), colony stimulating factor-1 (CSF-1R), and the basic fibroblast growth factor (bFGFR). C-Src is also overexpressed and activated in many of the same human carcinomas that overexpress EGFR and ErbB2. Src is also required for the maintenance of normal bone homeostasis through its regulation of osteoclast function.

p185erbB2

ErbB2 (Her2/neu) is a receptor tyrosine kinase overexpressed in a variety of malignancies including breast, ovarian, prostate, and gastric cancers. ErbB2 was originally identified as an oncogene and inhibition of Hsp90 results in the polyubiquitination and degradation of erbB2.

### Polo Mitotic Kinase

Polo-like kinases (Plks) are important regulators of cell cycle progression during M-phase. Plks are involved in the assembly of the mitotic spindle apparatus and in the activation of CDK/cyclin complexes. Plk1 regulates tyrosine dephosphorylation of CDKs through phosphorylation and activation of Cdc25C. CDK1 activation in turn leads to spindle formation and entry into M phase.

### Akt (PKB)

Akt is involved in pathways that regulate cell growth by stimulating cell proliferation and suppressing apoptosis. Hsp90 inhibition by ansamycins results in a reduction in the Akt half life through ubiquitination and proteasomal degradation. Binding of cdc37 to Hsp90 is also required for the down-regulation of Akt. Following ansamycin treatment cancer cells arrest in the G2/M phase of the cell cycle 24 hours after treatment and proceed to apoptosis 24-48 hours later. Normal cells also arrest 24 hours after ansamycin treatment, but do not proceed on to apoptosis.

c-Raf, B-RAF, Mek

The RAS-RAF-MEK-ERK-MAP kinase pathway mediates cellular responses to growth signals. RAS is mutated to an oncogenic form in approximately 15% of human cancers. The three RAF genes are serine/threonine kinases that are regulated by binding RAS.

### EGFR

The epidermal growth factor receptor (EGFR) is implicated in cell growth, differentiation, proliferation, survival, apoptosis, and migration. Overexpression of EGFR has been found in many different cancers and activating mutations of its kinase domain appear to be pathogenic in a subset of adenocarcinoams of the lung.

Flt3

FMS-like tyrosine kinase 3 (FLT3) is a receptor tyrosine kinase involved in cell proliferation, differentiation and apoptosis. Flt3 activation also leads to the activation of phosphatidylinositol 3-kinase (PI3K) and RAS signal-transduction cascades.

c-Met

c-met is a receptor tyrosine kinase which binds hepatocyte growth factor (HGF) and regulates both cell motility and cell growth. c-met is overexpressed in tumours, including thyroid, stomach, pancreatic and colon cancer. HGF is also detected around the tumours, including liver metastases. This suggests that c-met and HGF play an important role in invasion and metastasis.

### Cdk1, Cdk4, Cdk6

Cdk1, Cdk2, Cdk4, and Cdk6 drive the cell cycle. The activity of CDKs is regulated on by their binding to specific subunits such as cyclins, inhibitory and assembly factors. The substrate specificity and timing of CDK activities is dictated by their interaction with specific cyclins. Cdk4/cyclin D and Cdk6/cyclin D are active in the G1 phase, Cdk2/cyclin E and Cdk2/cyclin A in S phase, and Cdk2/cyclin A and Cdk2/cyclin B in G2/M phase.

Cyclin-dependent kinase type 4 (Cdk4), plays a key role in allowing cells to traverse G1 to S-phase transition of the cell cycle and is constitutively activated in many human cancers. The Cdk4 activator, cyclin D1, is overexpressed and a Cdk4 inhibitor, p16, is deleted in a variety of human tumours.

Cdk1/Cdk2 have been developed which reversibly block normal cells in either the G1/S-phase or at the G2/M border. G2/M arrest is generally less well tolerated by the cells and consequently, they undergo apoptotic cell death. Since Hsp90 also is known to affect cell survival pathways this effect may be further amplified with an Hsp90 inhibitor.

### Wee-1

The WEE1 protein kinase carries out the inhibitory phosphorylation of CDC2 on tyrosine 15 (Tyr15). This is required for activation of the G2-phase checkpoint in response to DNA damage.

Hsp90 transcription factors implicated in cell proliferation and survival include the following:

### Mutant p53

P53 is a tumour suppressor protein that causes cell cycle arrest and induces apoptosis. P53 is mutated in approximately half of all cancers. Mutant p53, associates with Hsp90 and, is down-regulated in cancer lines treated with Hsp90 inhibitors, while wild type p53 levels were unaffected.

### Progesterone Receptor/Estrogen Receptor/Androgen Receptor

In the absence of hormones, Progesterone and androgen receptors are bound by Hsp90 in an inactive form. Upon binding with their cognate hormones, the receptors undergo conformational changes and dissociation from hsp90. The ligand bound receptors are then capable of dimerisation, phosphorylation, and nuclear translocation. The activated receptors then bind to hormone-response elements (HREs) within the regulatory regions of target genes involved in maintaining cell proliferation.

### Hif-1a

Hypoxia inducible factor-1a (HIF-1a) is a transcription factor that controls the expression of genes which play a role in angiogenesis. HIF-1a is expressed in the majority of metastases and is known to associate with Hsp90. Ansamycin treatment of renal carcinoma cell lines leads to the ubiquitination and proteasomal degradation of HIF-1a.

Hsp90 inhibitors are capable of affecting a large number of targets significant to signal transduction in tumour cell proliferation. Signal transduction inhibitors which regulate the activities of a single target, may not be as efficacious due to signalling pathway redundancy and the rapid development of resistance.

By regulating multiple targets involved in cell signalling and cell proliferation HSP90 inhibitors may prove beneficial in the treatment of a wide spectrum of proliferative disorders.

### HERG

In the late 1990s a number of drugs, approved by the US FDA, had to be withdrawn from sale in the US when it was discovered they were implicated in deaths caused by heart malfunction. It was subsequently found that a side effect of these drugs was the development of arrhythmias caused by the blocking of hERG channels in heart cells. The hERG channel is one of a family of potassium ion channels the first member of which was identified in the late 1980s in a mutant Drosophila melanogaster fruitfly (see Jan, L. Y. and Jan, Y. N. (1990). A Superfamily of Ion Channels. Nature, 345(6277):672). The biophysical properties of the hERG potassium ion channel are described in Sanguinetti, M. C., Jiang, C., Curran, M. E., and Keating, M. T. (1995). A Mechanistic Link Between an Inherited and an Acquired Cardiac Arrhythmia: HERG encodes the Ikr potassium channel. Cell, 81:299-307, and Trudeau, M. C., Warmke, J. W., Ganetzky, B., and Robertson, G. A. (1995). HERG, a Human Inward Rectifier in the Voltage-Gated Potassium Channel Family. Science, 269:92-95.

The elimination of hERG blocking activity remains an important consideration in the development of any new drug.

### PRIOR ART

WO 03/075828 (Zetiq) discloses a class of aminopyrimidine compounds for use in treating cancer.

WO 02/36586 (AstraZeneca) discloses quinolinylaminopyrimidines for use in treating pain. Also disclosed are 6-substituted 2-amino-4-chloropyrimidines as chemical intermediates.

WO 01/62233 (Hoffmann La Roche) discloses substituted aminopyrimidines as adenosine receptor modulators useful in treating conditions and diseases such as pain, anxiety, Parkinson's disease and Alzheimer's disease.

WOO 2004/089286 (IRM LLC) discloses substituted pyrimidines having protein kinase inhibitory activity for use in treating proliferative disorders such as leukaemia.

WO 2004/017920 (University of Connecticut) discloses biphenyl and biphenyl-like cannabinoids for therapeutic use.

WO 03/106450 (Bayer) discloses phenylaminopyrimidines for treating cardiovascular disease.

WO 01/68612 (Cocensys) discloses aryl substituted pyrimidines as anticonvulsants.

WO 99/11633 (Boehringer Ingelheim) discloses triazines having adenosine receptor antagonist activity.

U.S. Pat. No. 5,863,924, U.S. Pat. No. 5,863,924 and U.S. Pat. No. 5,952,331 (all to Syntex) disclose aryl primidines as 5-HT_{2β} antagonists.

US 2004/204386 (Cell Therapeutics) discloses 4-anilino-6-phenylpyrimidine derivatives as LPAATβ inhibitors for the treatment of proliferative disorders. A number of phenyl)-2-pyrimidinamines are disclosed as synthetic intermediates.

WO 2004/039796 (Bayer) discloses 4-chloro-6-(2,4-difluorophenyl)-2-pyrimidinamine as a synthetic intermediate in the preparation of phenylaminopyrimidine Rho-Kinase II inhibitors intended for the treatment of cardiovascular disease.

WO 2002/096867 (LG Biomedical) discloses phenol and hydroxynaphthalene based compounds as protein kinase inhibitors. The compound 4-(5-bromo-2-methoxyphenyl)-6-chloro-2-pyrimidinamine is disclosed as a synthetic intermediate. Also disclosed are various 6-unsubstituted and substituted 2-pyrimidineamines bearing a biphenyl or heteroaryl-phenyl substituent at the 4-position thereof.

### SUMMARY OF THE INVENTION

The invention provides compounds that have Hsp90 inhibiting or modulating activity and which it is envisaged will be useful in preventing or treating disease states or conditions mediated by Hsp90.

Thus, for example, it is envisaged that the compounds of the invention will be useful in alleviating or reducing the incidence of cancer.

In a first aspect, the invention provides a compound for use as an inhibitor of Hsp90, the compound having the formula (I):

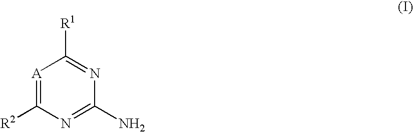

or salts, tautomers, solvates or N-oxides thereof; wherein:
A is N or a group CR³;
R¹ is a monocyclic or bicyclic carbocyclic or heterocyclic ring of 5 to 10 ring members of which up to two ring members may be heteroatoms selected from N, O and S and the remainder are carbon atoms, the carbocyclic or heterocyclic ring being optionally substituted by one or more substituent groups independently selected from R¹⁰;
R² is selected from:
hydrogen
halogen;
trifluoromethyl;
cyano;
amino;
mono- and di-C₁₋₄ hydrocarbylamino;
an acyclic C₁₋₁₀ hydrocarbyl group optionally substituted by one or more substituents independently selected from R¹¹ and wherein one or more carbon atoms of the acyclic C₁₋₁₀ hydrocarbyl group may optionally be replaced by O, S, SO, SO₂, NR_{C}, X¹C(X²), C(X²)X¹ or X¹C(X²)X¹;
a group R^{d}-R^{e} wherein R^{d} is O, CO, X¹C(X²), C(X²)X¹, X¹C(X²)X¹, S, SO, SO₂, NR^{c}, SO₂NR^{c} or NR^{c}SO₂; and R^{e} is selected from hydrogen, carbocyclic and heterocyclic groups having from 3 to 12 ring members, and a C₁₋₁₀ hydrocarbyl group optionally substituted by one or more substituents R¹, and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; provided that R^{d}-R^{e} is not hydroxy;
   R³ is selected from R² and monocyclic carbocyclic and heterocyclic groups having 3 to 7 ring members, wherein the monocyclic carbocyclic and heterocyclic groups are optionally substituted by one or more substituent groups independently selected from R¹⁰;
   R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₁₂ hydrocarbyl group (such as a C₁₋₁₀ hydrocarbyl group) optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; wherein the carbocyclic and heterocyclic groups of R¹⁰ may be unsubstituted or substituted by one or more further groups selected from R¹⁰, which further groups are not themselves further substituted;
   R^{c} is selected from R^{b}, hydrogen and C₁₋₄ hydrocarbyl; and

### X¹ is O, S or NR^{c} and X² is ═O, ═S or ═NR^{c}; and

R¹¹ is selected from hydroxy, oxo, halogen, cyano, nitro, carboxy, amino, mono- or di-C₁₋₄ hydrocarbylamino, carbocyclic and heterocyclic groups having from 3 to 12 ring members; wherein the carbocyclic and heterocyclic groups of R¹¹ may be unsubstituted or substituted by one or more further groups selected from R¹⁰.

In another aspect, the invention provides a compound for use in medicine having the formula (Ia):

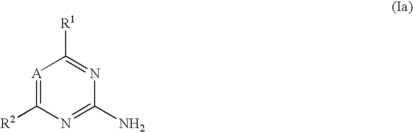

or salts, tautomers, solvates or N-oxides thereof; wherein:
A is N or a group CR³;
R¹ is a monocyclic or bicyclic carbocyclic or heterocyclic ring of 5 to 10 ring members of which up to two ring members may be heteroatoms selected from N, O and S and the remainder are carbon atoms, the carbocyclic or heterocyclic ring being optionally substituted by one or more substituent groups independently selected from R¹⁰;
R² is selected from:
hydrogen
halogen;
trifluoromethyl;
cyano;
amino;
mono- and di-C₁₋₄ hydrocarbylamino;
an acyclic C₁₋₁₀ hydrocarbyl group optionally substituted by one or more substituents R¹¹ and wherein one or more carbon atoms of the acyclic C₁₋₁₀ hydrocarbyl group may optionally be replaced by O, S, SO, SO₂, NR^{c}, X¹C(X²), C(X²)X¹ or X¹C(X²)X¹;
a group R^{d}-R^{e} wherein R^{d} is O, CO, X¹C(X²), C(X²)X¹, X¹C(X²)X¹, S, SO, SO₂, NR^{c}, SO₂NR^{c} or NR^{c}SO₂; and R^{e} is selected from hydrogen, carbocyclic and heterocyclic groups having from 3 to 12 ring members, and a C₁₋₁₀ hydrocarbyl group optionally substituted by one or more substituents R¹¹, and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; provided that R^{d}-R^{e} is not hydroxy;
   R³ is selected from R² and monocyclic carbocyclic and heterocyclic groups having 3 to 7 ring members, wherein the monocyclic carbocyclic and heterocyclic groups are optionally substituted by one or more substituent groups independently selected from R¹⁰;
   R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₁₂ hydrocarbyl group (such as a C₁₋₁₀ hydrocarbyl group) optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; wherein the carbocyclic and heterocyclic groups of R¹⁰ may be unsubstituted or substituted by one or more further groups selected from R¹⁰, which further groups are not themselves further substituted;
   R^{c} is selected from R^{b}, hydrogen and C₁₋₄ hydrocarbyl; and

### X¹ is O, S or NR^{c} and X² is ═O, ═S or ═NR^{c}; and

R¹¹ is selected from hydroxy, oxo, halogen, cyano, nitro, carboxy, amino, mono- or di-C₁₋₄ hydrocarbylamino, carbocyclic and heterocyclic groups having from 3 to 12 ring members; wherein the carbocyclic and heterocyclic groups of R¹¹ may be unsubstituted or substituted by one or more further groups selected from R¹⁰; provided that the compound is other than:
(a-i) the compounds 4-chloro-6-phenyl-pyrimidin-2-ylamine, 4-(5-methyl-3-phenyl-isoxazol-4-yl)-pyrimidin-2-ylamine, 4-(2-thienyl)-2-pyrimidinamine, 2-amino-4-phenyl-6-methyl-1,3,5-triazine; 2-amino-4-(carboxymethyl)-6-phenyl-1,3,5-triazine; 2-amino-4-cyano-6-phenyl-1,3,5-triazine; 2-amino-4-phenylamino-6-phenyl-1,3,5-triazine; 2-amino-4-(carboxymethyl)-6-phenyl-1,3,5-triazine;
(a-ii) a compound wherein R² is an optionally substituted quinolinylamino group and R¹ is 4-fluorophenyl or 4-chlorophenyl;
(a-iii) a compound wherein A is CR³ wherein R³ is cyano or halogen;
(a-iv) a compound wherein R¹ is unsubstituted phenyl and R² is an acyclic hydrocarbyl group linked to an optionally substituted pyridyl group;
(a-v) a compound wherein R¹ is unsubstituted 3-pyridyl and R² is other than chlorine, amino or methyl;
(a-vi) a compound wherein R¹ is optionally substituted naphthyl, indolyl, quinolinyl or isoquinolinyl;
(a-vii) a compound wherein A is N, R² is amino and R¹ is a 3-phenoxyphenyl or 3-phenylsulphanylphenyl group;
(a-viii) a compound wherein A is CR³, R¹ is optionally substituted furanyl and R² contains a pyridylalkyl moiety;
(a-ix) a compound wherein A is CR³, R² is an optionally substituted alkyl group and R¹ is an optionally substituted bicyclic group;
(a-x) a compound wherein A is CR³, R² is alkyl and R¹ is an indolyl group;
(a-xi) a compound wherein A is CR³, R² is methyl and R¹ is selected from 5-chlorothiophen-2-yl, 3-fluorophenyl, 3-chlorophenyl, 3-methoxyphenyl, 3-n-triphenyl, 3-chloro-4-fluorophenyl, 3,5-difluorophenyl, 3,5-dichlorophenyl, 4-amino-5-chloro-2-methoxphenyl and 3-trifluoromethylphenyl;
(a-xii) a compound wherein A is CR³, R² is isopropyl and R¹ is 4-amino-5-chloro-2-methoxphenyl;
(a-xiii) a compound wherein R¹ is optionally substituted quinolinyl or dihydroquinolinyl;
(a-xiv) a compound wherein R¹ is a 2-hydroxy-5-aroyl-phenyl or 2-hydroxy-5-heteroaroyl-phenyl group;
(a-xv) a compound wherein A is CH, R² is hydrogen, and R¹ is other than a 3-pyridyl group substituted at the 2-position thereof with an aryl or heteroaryl group, and wherein the 4-, 5- and 6-positions of the 3-pyridyl group are optionally substituted;
(a-xvi) the compound irsogladine (6-(2,5-dichloro-phenyl)-[1,3,5]triazine-2,4-diamine; and
(a-xvii) a compound wherein A is N and R² is a substituted or unsubstituted amino group, alkanoylamino group or a saturated 5- or 6-membered heterocyclic group.

In a further aspect, the invention provides a novel compound per se of the formula (Ib):

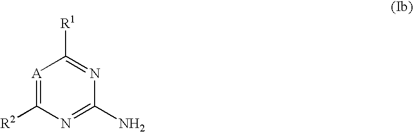

or salts, tautomers, solvates or N-oxides thereof; wherein:
A is N or a group CR³;
R¹ is a monocyclic or bicyclic carbocyclic or heterocyclic ring of 5 to 10 ring members of which up to two ring members may be heteroatoms selected from N, O and S and the remainder are carbon atoms, the carbocyclic or heterocyclic ring being optionally substituted by one or more substituent groups independently selected from R₁₀;
R² is selected from:
hydrogen
halogen;
trifluoromethyl;
cyano;
amino;
mono- and di-C₁₋₄ hydrocarbylamino;
an acyclic C₁₋₁₀ hydrocarbyl group optionally substituted by one or more substituents R¹¹ and wherein one or more carbon atoms of the acyclic C₁₋₁₀ hydrocarbyl group may optionally be replaced by O, S, SO, SO₂, NR^{c}, X¹C(X²), C(X²)X¹ or X¹C(X²)X¹;
a group R^{d}-R^{e} wherein R¹ is O, CO, X¹C(X²), C(X²)X, X¹C(X²)X¹, S, SO, SO₂, NR^{c}, SO₂NR^{c} or NR^{c}SO₂; and R^{e} is selected from hydrogen, carbocyclic and heterocyclic groups having from 3 to 12 ring members, and a C₁₋₁₀ hydrocarbyl group optionally substituted by one or more substituents R¹¹, and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; provided that R^{d}-R^{e} is not hydroxy;
   R³ is selected from R² and monocyclic carbocyclic and heterocyclic groups having 3 to 7 ring members, wherein the monocyclic carbocyclic and heterocyclic groups are optionally substituted by one or more substituent groups independently selected from R¹⁰;
   R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR_{c}SO₂; and R^{b} is selected from hydrogen, carbocyclic and heterocyclic groups having from 3 to 12 ring members, and a C₁₋₁₂ hydrocarbyl group (such as a C₁₋₁₀ hydrocarbyl group) optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; wherein the carbocyclic and heterocyclic groups of R¹⁰ may be unsubstituted or substituted by one or more further groups selected from R¹⁰, which further groups are not themselves further substituted;
   R^{c} is selected from R^{b}, hydrogen and C₁₋₄ hydrocarbyl; and

### X¹ is O, S or NR^{c} and X² is ═O, ═S or ═NR^{c}; and

R¹¹ is selected from hydroxy, oxo, halogen, cyano, nitro, carboxy, amino, mono- or di-C₁₋₄ hydrocarbylamino, carbocyclic and heterocyclic groups having from 3 to 12 ring members; wherein the carbocyclic and heterocyclic groups of R¹¹ may be unsubstituted or substituted by one or more further groups selected from R¹⁰; provided that the compound is other than:
(a-i) the compounds 4-chloro-6-phenyl-pyrimidin-2-ylamine, 4-(5-methyl-3-phenyl-isoxazol-4-yl)-pyrimidin-2-ylamine, 4-(2-thienyl)-2-pyrimidinamine, 2-amino-4-phenyl-6-methyl-1,3,5-triazine; 2-amino-4-(carboxymethyl)-6-phenyl-1,3,5-triazine; 2-amino-4-cyano-6-phenyl-1,3,5-triazine; 2-amino-4-phenylamino-6-phenyl-1,3,5-triazine; 2-amino-4-(carboxymethyl)-6-phenyl-1,3,5-triazine;
(a-ii) a compound wherein R² is an optionally substituted quinolinylamino group and R¹ is 4-fluorophenyl or 4-chlorophenyl;
(a-iii) a compound wherein A is CR³ wherein R³ is cyano or halogen;
(a-iv) a compound wherein R¹ is unsubstituted phenyl and R² is an acyclic hydrocarbyl group linked to an optionally substituted pyridyl group;
(a-v) a compound wherein R¹ is unsubstituted 3-pyridyl and R² is other than chlorine, amino or methyl;
(a-vi) a compound wherein R¹ is optionally substituted naphthyl or indolyl;
(a-vii) a compound wherein A is N, R² is amino and R¹ is a 3-phenoxyphenyl or 3-phenylsulphanylphenyl group;
(a-viii) a compound wherein A is CR³, R¹ is optionally substituted furanyl and R² contains a pyridylalkyl moiety;
(a-ix) a compound wherein A is CR³, R² is methyl and R¹ is an optionally substituted bicyclic group;
(a-x) a compound wherein A is CR³, R² is alkyl and R¹ is an indolyl group;
(a-xi) a compound wherein A is CR³, R² is methyl and R¹ is selected from 3-fluorophenyl, 3-chlorophenyl, 3-methoxyphenyl, 3-n-triphenyl, 3-chloro-4-fluorophenyl, 3,5-difluorophenyl, 4-amino-5-chloro-2-methoxphenyl and 3-trifluoromethylphenyl;
(a-xii) a compound wherein A is CR³, R² is isopropyl and R¹ is 4-amino-5-chloro-2-methoxphenyl;
(a-xiii) a compound wherein R¹ is optionally substituted dihydroquinolinyl;
(a-xiv) a compound wherein R¹ is a 2-hydroxy-5-aroyl-phenyl or 2-hydroxy-5-heteroaroyl-phenyl group;
(a-xv) a compound wherein A is CH, R² is hydrogen, and R¹ is other than a 3-pyridyl group substituted at the 2-position thereof with an aryl or heteroaryl group, and wherein the 4-, 5- and 6-positions of the 3-pyridyl group are optionally substituted;
(a-xvi) the compound irsogladine (6-(2,5-dichloro-phenyl)-[1,3,5]triazine-2,4-diamine;
(a-xvii) a compound wherein A is N and R² is a substituted or unsubstituted amino group, alkanoylamino group or a saturated 5- or 6-membered heterocyclic group;
(b-i) 4-chloro-6-(4-fluorophenyl)pyrimidinyl-2-amine and 4-chloro-6-(4-chlorophenyl)pyrimidinyl-2-amine;
(b-ii) 2,4-diamino-4-phenyltriazine;
(b-iii) 2-benzoylamino-6-amino-4-phenyltriazine;
(b-iv) 2-amino-4-n-butylamino-6-phenyl-[1,3,5]-triazine;
(b-v) 6-{4-[ethyl-(2-methoxy-ethyl)-amino]-2-methyl-phenyl}-[1,3,5]triazine-2,4-diamine;
(b-vi) a compound wherein A is N and R² is C₁₋₄ alkoxy or C₁₋₂ alkoxy-C₁₋₂ alkoxy;
(b-vii) 4-methylsulphanyl-6-(4-phenoxy-phenyl)-[1,3,5]triazin-2-ylamine;
(b-viii) a compound wherein R¹ is methanesulphinyl;
(b-ix) a compound wherein R¹ is furanyl;
(b-x) a compound wherein R¹ is a phenyl group bearing a hydroxy or benzyloxy substituent at the 2-position thereof and an optionally substituted aryl, heteroaryl, amide, ester, aroyl or heteroaroyl group at the 5-position thereof;
(b-xi) a compound wherein R¹ is a phenyl group bearing a hydroxy or benzyloxy substituent at the 4-position thereof and an optionally substituted aryl or heteroaryl group at the 3-position thereof;
(b-xii) a compound wherein R¹ is a 2,5-disubstituted phenyl group bearing an alkyl, halogen or alkoxy substituent at the 2-position thereof and a halogen or alkoxy substituent at the 5-position thereof;
(b-xiii) a compound wherein R² is chlorine and R¹ is a dimethoxyphenyl or trimethoxyphenyl group; and
(b-xiv) the compounds:
4-chloro-6-(2,3,5-trichlorophenyl)-2-pyrimidinamine;
4-chloro-6-(2,4-difluorophenyl)-2-pyrimidinamine;
4-(4-amino-5-chloro-2-methoxyphenyl)-6-methyl-2-pyrimidinamine;
4-chloro-6-(2-methylphenyl)-2-pyrimidinamine;
4-chloro-6-(2-methoxyphenyl)-2-pyrimidinamine;
4-(2-chlorophenyl)-6-ethoxy-2-pyrimidinamine.

The invention also provides inter alia:
A compound of the formula (I), (Ia), (Ib), (II), (IIIa), (III), (IV), (IVa), (V), (VI), (VII) or (VIII) or any sub-groups or examples thereof as defined herein for use in the prophylaxis or treatment of a disease state or condition mediated by Hsp90.
The use of a compound of the formula (I), (Ia), (Ib), (II), (Ia), (III), (IV), (IVa), (V), (VI), (VII) or (VIII) or any sub-groups or examples thereof as defined herein for the manufacture of a medicament for the prophylaxis or treatment of a disease state or condition mediated by Hsp90.
A method for the prophylaxis or treatment of a disease state or condition mediated by Hsp90, which method comprises administering to a subject in need thereof a compound of the formula (I), (Ia), (Ib), (II), (IIa), (III), (IV), (IVa), (V), (VI), (VII) or (VIII) or any sub-groups or examples thereof as defined herein.
A compound of the formula (I), (Ia), (Ib), (II), (IIa), (III), (IV), (IVa), (V), (VI), (VII) or (VIII) or any sub-groups or examples thereof as defined herein for use in alleviating or reducing the incidence of a disease state or condition mediated by Hsp90.
The use of a compound of the formula (I), (Ia), (Ib), (II), (IIa), (III), (IV), (IVa), (V), (VI), (VII) or (VIII) or any sub-groups or examples thereof as defined herein for the manufacture of a medicament for alleviating or reducing the incidence of a disease state or condition mediated by Hsp90.
A method for alleviating or reducing the incidence of a disease state or condition mediated by Hsp90, which method comprises administering to a subject in need thereof a compound of the formula (I), (Ia), (Ib), (II), (Ia), (III), (IV), (IVa), (V), (VI), (VII) or (VIII) or any sub-groups or examples thereof as defined herein.

A compound of the formula (I), (Ia), (Ib), (II), (IIa), (III), (IV), (IVa), (V), (VI), (VII) or (VIII) or any sub-groups or examples thereof as defined herein for use in treating a disease or condition comprising or arising from abnormal cell growth in a mammal.

The use of a compound of the formula (I), (Ia), (Ib), (II), (IIa), (III), (IV), (IVa), (V), (VI), (VII) or (VIII) or any sub-groups or examples thereof as defined herein for the manufacture of a medicament for treating a disease or condition comprising or arising from abnormal cell growth in a mammal.
A method for treating a disease or condition comprising or arising from abnormal cell growth in a mammal, which method comprises administering to the mammal a compound of the formula (I), (Ia), (Ib), (II), (IIa), (III), (IV), (IVa), (V), (VI), (VII) or (VIII) or any sub-groups or examples thereof as defined herein in an amount effective in inhibiting abnormal cell growth.
A compound of the formula (I), (Ia), (Ib), (II), (Ia), (III), (IV), (IVa), (V), (VI), (VII) or (VIII) or any sub-groups or examples thereof as defined herein for use in alleviating or reducing the incidence of a disease or condition comprising or arising from abnormal cell growth in a mammal.

The use of a compound of the formula (I), (Ia), (Ib), (II), (IIa), (III), (IV), (IVa), (V), (VI), (VII) or (VIII) or any sub-groups or examples thereof as defined herein for the manufacture of a medicament for alleviating or reducing the incidence of a disease or condition comprising or arising from abnormal cell growth in a mammal.
A method for alleviating or reducing the incidence of a disease or condition comprising or arising from abnormal cell growth in a mammal, which method comprises administering to the mammal a compound of the formula (I), (Ia), (Ib), (II), (IIa), (III), (IV), (IVa), (V), (VI), (VII) or (VIII) or any sub-groups or examples thereof as defined herein in an amount effective in inhibiting abnormal cell growth.
A method for treating a disease or condition comprising or arising from abnormal cell growth in a mammal, the method comprising administering to the mammal a compound of the formula (I), (Ia), (Ib), (II), (IIa), (III), (IV), (IVa), (V), (VI), (VII) or (VIII) or any sub-groups or examples thereof as defined herein in an amount effective to inhibit Hsp90 activity.
A method for alleviating or reducing the incidence of a disease or condition comprising or arising from abnormal cell growth in a mammal, the method comprising administering to the mammal a compound of the formula (I), (Ia), (Ib), (II), (IIa), (III), (IV), (IVa), (V), (VI), (VII) or (VIII) or any sub-groups or examples thereof as defined herein in an amount effective to inhibit Hsp90 activity.
A compound of the formula (I), (Ia), (Ib), (II), (IIa), (III), (IV), (IVa), (V), (VI), (VII) or (VIII) or any sub-groups or examples thereof as defined herein for use as an inhibitor of Hsp90.
A method of inhibiting Hsp90, which method comprises contacting the Hsp90 with an Hsp90-inhibiting compound of the formula (I), (Ia), (Ib), (II), (Ia), (III), (IV), (IVa), (V), (VI), (VII) or (VIII) or any sub-groups or examples thereof as defined herein.
A compound of the formula (I), (Ia), (Ib), (II), (IIa), (III), (IV), (IVa), (V), (VI), (VII) or (VIII) or any sub-groups or examples thereof as defined herein for use in modulating a cellular process (for example cell division) by inhibiting the activity of Hsp90.
A method of modulating a cellular process (for example cell division) by inhibiting the activity of Hsp90 using a compound of the formula (I), (Ia), (Ib), (II), (IIa), (III), (IV), (IVa), (V), (VI), (VII) or (VIII) or any sub-groups or examples thereof as defined herein.
A compound of the formula (I), (Ia), (Ib), (II), (IIa), (III), (IV), (IVa), (V), (VI), (VII) or (VIII) or any sub-groups or examples thereof as defined herein for use in the prophylaxis or treatment of a disease state as described herein.
The use of a compound of the formula (I), (Ia), (Ib), (II), (IIa), (III), (IV), (IVa), (V), (VI), (VII) or (VIII) or any sub-groups or examples thereof as defined herein for the manufacture of a medicament, wherein the medicament is for any one or more of the uses defined herein.
A pharmaceutical composition comprising a compound of the formula (I), (Ia), (Ib), (II), (IIa), (III), (IV), (IVa), (V), (VI), (VII) or (VIII) or any sub-groups or examples thereof as defined herein and a pharmaceutically acceptable carrier.
A pharmaceutical composition comprising a compound of the formula (I), (Ia), (Ib), (II), (IIa), (III), (IV), (IVa), (V), (VI), (VII) or (VIII) or any sub-groups or examples thereof as defined herein and a pharmaceutically acceptable carrier in a form suitable for oral administration.
A pharmaceutical composition comprising a compound of the formula (I), (Ia), (Ib), (II), (IIa), (III), (IV), (IVa), (V), (VI), (VII) or (VIII) or any sub-groups or examples thereof as defined herein and a pharmaceutically acceptable carrier in a form suitable for parenteral administration, for example by intravenous (i.v.) administration.
A pharmaceutical composition comprising a compound of the formula (I), (Ia), (Ib), (II), (IIa), (III), (IV), (IVa), (V), (VI), (VII) or (VIII) or any sub-groups or examples thereof as defined herein and a pharmaceutically acceptable carrier in a form suitable for intravenous (i.v.) administration by injection or infusion.
A compound of the formula (I), (Ia), (Ib), (II), (IIa), (III), (IV), (IVa), (V), (VI), (VII) or (VIII) or any sub-groups or examples thereof as defined herein for use in medicine.
A compound as defined herein for any of the uses and methods set forth above, and as described elsewhere herein.

A compound of the formula (I), (Ia), (Ib), (II), (IIa), (III), (IV), (IVa), (V), (VI), (VII) or (VIII) or any sub-groups or examples thereof as defined herein for use in treatment or prophylaxis of a disease state or condition in a patient who has been screened and has been determined as suffering from, or being at risk of suffering from, a disease or condition which would be susceptible to treatment with a compound having activity against Hsp90.
The use of a compound of the formula (I), (Ia), (Ib), (II), (IIa), (III), (IV), (IVa), (V), (VI), (VII) or (VIII) or any sub-groups or examples thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against Hsp90.
A method for the diagnosis and treatment of a disease state or condition mediated by Hsp90, which method comprises (i) screening a patient to determine whether a disease or condition from which the patient is or may be suffering is one which would be susceptible to treatment with a compound having activity against Hsp90; and (ii) where it is indicated that the disease or condition from which the patient is thus susceptible, thereafter administering to the patient a compound of the formula (I), (Ia), (Ib), (II), (IIa), (III), (IV), (IVa), (V), (VI), (VII) or (VIII) or any sub-groups or examples thereof as defined herein.

Where they do not already apply, any one or more of the following optional provisos may apply in any combination to each of the formulae (I), (Ia), (Ib), (II), (Ia), (III), (IV), (IVa), (V), (VI), (VII) or (VIII) and sub-groups and embodiments thereof as defined herein unless the context indicates otherwise.

Any one or more, in any combination, of provisos (a-i) to (a-xvii) above.

Any one or more, in any combination, of provisos (b-i) to (b-xiv) above.

Any one of more, in any combination, of provisos (c-i) to (c-xxv) as follows:

(c-i) R² is other than a substituted or unsubstituted arylthio, aryloxy, heteroaryl thio or heteroaryloxy group.

(c-ii) R² is other than an optionally substituted phenylamino group wherein the phenyl moiety of the phenylamino group is linked to an optionally substituted pyridylthio, pyridylsulphinyl or quinolinyloxy group.

(c-iii) R² is other than an optionally substituted quinolinylamino group.

(c-iv) R¹ is other than 4-fluorophenyl.

(c-v) R¹ is other than 4-chlorophenyl.

(c-yl) R¹ is other than 4-fluorophenyl when R² is chloro.

(c-vii) R¹ is other than 4-chlorophenyl when R² is chloro.

(c-viii) When A is N, R¹ is other than a 2-hydroxyphenyl phenyl group optionally bearing another substituent.

(c-ix) When A is N, R¹ is other than an aryl or heteroaryl group bearing a hydroxy group ortho to the point of attachment to the A-containing ring.

(c-x) A is other than CR³ where R³ is a cyano group.

(c-xi) R¹ is other than a phenyl group bearing one or more substituents of which one is apara substituent containing an optionally substituted piperazine group.

(c-xii) When A is N and R² is amino or alkoxy, R¹ is other than a xylyl group.

(c-xiii) R² is other than a substituted or unsubstituted arylamino group.

(c-xiv) R¹ is other than an optionally substituted diaminophenyl group.

(c-xv) R¹ is other than unsubstituted 3-pyridyl.

(c-xvi) R² is other than cyclohexylamino.

(c-xvii) when A is N, R² is other than optionally substituted amino.

(c-xviii) when A is N, R¹ and R² are each other than a group comprising an optionally substituted para-aminophenyl moiety wherein the amino component of the para-aminophenyl moiety together with one or more substituent groups thereon and/or one or more carbon atoms of the phenyl ring of the aminophenyl moiety may optionally form part of one or two further ring systems.

(c-xix) R¹ is other than unsubstituted phenyl, tolyl, xylyl or mesityl.

(c-xx) When R² is selected from hydrogen, optionally substituted alkyl, alkoxy, acyl, alkenyl, amino, dialkylamino, carboxyl, alkoxycarbonyl, carboxyalkyl, carbamoyl, benzyl, styryl, cinnamyl, mercapto, cyano and halogen, R¹ is other than unsubstituted phenyl, tolyl, xylyl or mesityl.

(c-xxi) R¹ is other than an optionally substituted phenoxyphenyl, phenylsulphanylphenyl, phenylaminophenyl or benzylphenyl group;

(c-xxii) When R² is aminocarbonyl, R¹ is other than an optionally substituted phenyl ring bearing one or more substituents wherein at least one of the said substituents is selected from optionally substituted alkoxy, optionally substituted alkylsulphanyl, optionally substituted alkylamino or optionally substituted alkyl.

(c-xxiii) R² is other than methylsulphanyl, methylsulphonyl and trifluoromethanesulphonyloxy.

(c-xxiv) R¹ is other than an optionally substituted tetrahydroquinoline group.

(c-xxv) R¹ is other than a phenyl group bearing a hydroxy or benzyloxy group at the 2-position thereof and a carbonyl group-containing substituent at the 5-position thereof, wherein the carbonyl group is directly attached to the phenyl group.

Furthermore, each of the formulae herein relating to compounds per se explicitly exclude from the scope thereof any specific compounds disclosed in the documents identified as (d-i) to (d-xxviii) below, the contents of each of which are incorporated herein by reference.
(d-i) US 2004/204386
(d-ii) WO 2005/108397
(d-iii) WO 2005/033085
(d-iv) WO 2004/039796
(d-v) WO 2002/096867
(d-vi) U.S. Pat. No. 5,958,934
(d-vii) U.S. Pat. No. 5,863,924
(d-viii) U.S. Pat. No. 5,952,331
(d-ix) WO 97/44326
(d-x) Biorganic & Medicinal Chemistry Letters (2005), 15(16), 3670-74
(d-xi) Tetrahedron, (1979), 35(4), 551-6
(d-xii) WO 2005/051366
(d-xiii) WO2005/108397
(d-xiv) WO2005/033085
(d-xv) WO2002/096886
(d-xvi) WO99/42455
(d-xvii) WO2004/089286
(d-xviii) US2004/118317
(d-xix) WO2004/054986
(d-xx) WO2004/039786
(d-xxi) WO2004/017920
(d-xxii) WO2003/075828
(d-xxiii) WO2002/036586
(d-xxiv) WO2001/062233
(d-xxv) U.S. Pat. No. 5,863,924
(d-xxvi) WO97/44326
(d-xxvii) EP742212
(d-xxviii) EP397474

Moreover, each of the specific compounds disclosed in any of the above documents (d-i) to (d-xxviii) may serve as the basis for an additional optional proviso excluding the specific compound from the scope of any one or more of formulae (I), (Ia), (Ib), (II), (IIa), (III), (IV), (IVa), (V), (VI), (VII) or (VIII) and sub-groups and embodiments thereof as defined herein, and such additional provisos may apply in any combination to any of the said formulae.

### GENERAL PREFERENCES AND DEFINITIONS

In this section, as in all other sections of this application, unless the context indicates otherwise, references to a compound of formula (I) includes all subgroups of formula (I) as defined herein, including formulae (Ia), (Ib), (II), (IIa), (III), (IV), (IVa), (V), (VI), (VII) or (VIII) and the term ‘subgroups’ includes all preferences, embodiments, examples and particular compounds defined herein.

Moreover, a reference to a compound of formula (I) and sub-groups thereof includes ionic forms, salts, solvates, isomers, tautomers, N-oxides, esters, prodrugs, isotopes and protected forms thereof, as discussed below:—preferably, the salts or tautomers or isomers or N-oxides or solvates thereof:—and more preferably, the salts or tautomers or N-oxides or solvates thereof.

The following general preferences and definitions shall apply to each of R¹ to R³, R¹⁰, R¹¹, R^{a}, R^{b}, R^{c}, R^{d}, R^{e}, X¹ and X² and their various sub-groups, sub-definitions, examples and embodiments unless the context indicates otherwise.

Any references to formula (I) herein shall also be taken to refer to and any sub-group of compounds within formula (I) and any preferences and examples thereof unless the context requires otherwise.

In this application, the moiety:

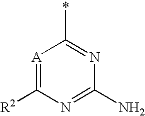

in which the asterisk shows the point of attachment of the monocyclic or bicyclic group R¹, may be referred to for convenience as the “pyrimidine or triazine ring” or the “pyrimidine or triazine moiety” or the “pyrimidine or triazine group”.

Where references are made to the possibility of one or more substituents being present on a particular moiety (e.g. a ring or a hydrocarbyl group) and a list of possible substituents is given, it is to be understood that the substituents present on the moiety are independently selected from the list and hence can be the same or different, unless the context indicates otherewise.

References to “carbocyclic” and “heterocyclic” groups as used herein shall, unless the context indicates otherwise, include both aromatic and non-aromatic ring systems. Thus, for example, the term “carbocyclic and heterocyclic groups” includes within its scope aromatic, non-aromatic, unsaturated, partially saturated and fully saturated carbocyclic and heterocyclic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, for example 5 to 7, and preferably 5 or 6 ring members. Examples of bicyclic groups are those containing 8, 9, 10, 11 and 12 ring members, and more usually 9 or 10 ring members.

The term “bicyclic” as used herein refers to groups that have two rings joined together in such as way that at least one ring member is shared by both rings. Thus, the bicyclic group can be a fused ring (two ring members shared by both rings), spirocyclic (one ring member shared by both rings) or a bridged ring (three or more ring members shared by both rings).

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or substituted with one or more substituents, for example one or more groups R¹⁰ as defined herein.

The term “aroyl” as used herein (see for example proviso (a-xiv) above) refers to a group of the formula —C(O)—Ar where Ar is a substituted or unsubstituted aryl group such as substituted or unsubstituted phenyl group. Analogously, the term “heteroaroyl” as used herein refers to a group of the formula —C(O)—Het where Het is a substituted or unsubstituted heteroaryl group.

The term “non-aromatic group” embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C≡C or N═C bond. The terms “fully saturated” and “saturated” refer to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl. A further example of a cycloalkenyl group is cyclohexenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings or, by way of a further example, two fused five membered rings. Each ring may contain up to about four heteroatoms typically selected from nitrogen, sulphur and oxygen. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a a 5- or 6-membered ring containing 1, 2 or 3 ring heteroatoms;
e) a pyrazole ring fused to a a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

Particular examples of bicyclic heteroaryl groups containing a five membered ring fused to another five membered ring include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring include but are not limited to benzfuran, benzthiophene, benzimidazole, benzoxazole, isobenzoxazole, benzisoxazole, benzthiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), triazolopyrimidine (e.g. [1,2,4]triazolo[1,5-a]pyrimidine), benzodioxole and pyrazolopyridine (e.g. pyrazolo[1,5-a]pyridine) groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

One sub-group of heteroaryl groups comprises pyridyl, pyrrolyl, furanyl, thienyl, imidazolyl, oxazolyl, oxadiazolyl, oxatriazolyl, isoxazolyl, thiazolyl, isothiazolyl, pyrazolyl, pyrazinyl, pyridazinyl, pyrimidinyl, triazinyl, triazolyl, tetrazolyl, quinolinyl, isoquinolinyl, benzfuranyl, benzthienyl, chromanyl, thiochromanyl, benzimidazolyl, benzoxazolyl, benzisoxazole, benzthiazolyl and benzisothiazole, isobenzofuranyl, indolyl, isoindolyl, indolizinyl, indolinyl, isoindolinyl, purinyl (e.g., adenine, guanine), indazolyl, benzodioxolyl, chromenyl, isochromenyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

Examples of polycyclic aryl and heteroaryl groups containing an aromatic ring and a non-aromatic ring include tetrahydronaphthalene, tetrahydroisoquinoline, tetrahydroquinoline, dihydrobenzthiene, dihydrobenzfuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups include unsubstituted or substituted (by one or more groups R¹⁰) heterocyclic groups having from 3 to 12 ring members, typically 4 to 12 ring members, and more usually from 5 to 10 ring members. Such groups can be monocyclic or bicyclic, for example, and typically have from 1 to 5 heteroatom ring members (more usually 1, 2, 3 or 4 heteroatom ring members) typically selected from nitrogen, oxygen and sulphur.

When sulphur is present, it may, where the nature of the adjacent atoms and groups permits, exist as —S—, —S(O)— or —S(O)₂—.

The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic amide moieties (e.g. as in pyrrolidone), cyclic thioamides, cyclic thioesters, cyclic ester moieties (e.g. as in butyrolactone), cyclic sulphones (e.g. as in sulpholane and sulpholene), cyclic sulphoxides, cyclic sulphonamides and combinations thereof (e.g. morpholine and thiomorpholine and its S-oxide and S,S-dioxide). Further examples of heterocyclic groups are those containing a cyclic urea moiety (e.g. as in imidazolidin-2-one),

In one sub-set of heterocyclic groups, the heterocyclic groups contain cyclic ether moieties (e.g as in tetrahydrofuran and dioxane), cyclic thioether moieties (e.g. as in tetrahydrothiophene and dithiane), cyclic amine moieties (e.g. as in pyrrolidine), cyclic sulphones (e.g. as in sulpholane and sulpholene), cyclic sulphoxides, cyclic sulphonamides and combinations thereof (e.g. thiomorpholine).

Examples of monocyclic non-aromatic heterocyclic groups include 5-, 6- and 7-membered monocyclic heterocyclic groups. Particular examples include morpholine, piperidine (e.g. 1-piperidinyl, 2-piperidinyl, 3-piperidinyl and 4-piperidinyl), pyrrolidine (e.g. 1-pyrrolidinyl, 2-pyrrolidinyl and 3-pyrrolidinyl), pyrrolidone, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazine, and N-alkyl piperazines such as N-methyl piperazine. Further examples include thiomorpholine and its S-oxide and S,S-dioxide (particularly thiomorpholine). Still further examples include azetidine, piperidone, piperazone, and N-alkyl piperidines such as N-methyl piperidine.

One preferred sub-set of non-aromatic heterocyclic groups consists of saturated groups such as azetidine, pyrrolidine, piperidine, morpholine, thiomorpholine, thiomorpholine S,S-dioxide, piperazine, N-alkyl piperazines, and N-alkyl piperidines.

Another sub-set of non-aromatic heterocyclic groups consists of pyrrolidine, piperidine, morpholine, thiomorpholine, thiomorpholine S,S-dioxide, piperazine and N-alkyl piperazines such as N-methyl piperazine.

One particular sub-set of heterocyclic groups consists of pyrrolidine, piperidine, morpholine and N-alkyl piperazines (e.g. N-methyl piperazine), and optionally thiomorpholine.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Preferred non-aromatic carbocyclic groups are monocyclic rings and most preferably saturated monocyclic rings.

Typical examples are three, four, five and six membered saturated carbocyclic rings, e.g. optionally substituted cyclopentyl and cyclohexyl rings.

One sub-set of non-aromatic carbocylic groups includes unsubstituted or substituted (by one or more groups R¹⁰ which may the same or different) monocyclic groups and particularly saturated monocyclic groups, e.g. cycloalkyl groups. Examples of such cycloalkyl groups include cyclopropyl, cyclobutyl, cyclopentyl, cyclohexyl and cycloheptyl; more typically cyclopropyl, cyclobutyl, cyclopentyl and cyclohexyl, particularly cyclohexyl.

Further examples of non-aromatic cyclic groups include bridged ring systems such as bicycloalkanes and azabicycloalkanes although such bridged ring systems are generally less preferred. By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged ring systems include bicyclo[2.2.1]heptane, aza-bicyclo[2.2.1]heptane, bicyclo[2.2.2]octane, aza-bicyclo[2.2.2]octane, bicyclo[3.2.1]octane and aza-bicyclo[3.2.1]octane. A particular example of a bridged ring system is the 1-aza-bicyclo[2.2.2]octan-3-yl group.

Where reference is made herein to carbocyclic and heterocyclic groups, the carbocyclic or heterocyclic ring can, unless the context indicates otherwise, be unsubstituted or substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₁₂ hydrocarbyl group (such as a C₁₋₁₀ hydrocarbyl group) optionally substituted by one or more substituents selected from hydroxy, oxo, halogen, cyano, nitro, carboxy, amino, mono- or di-C₁₋₈ non-aromatic hydrocarbylamino (e.g. mono- or di-C₁₋₄ hydrocarbylamino), carbocyclic and heterocyclic groups having from 3 to 12 ring members and wherein one or more carbon atoms of the C₁₋₁₂ hydrocarbyl group (or C₁₋₁₀ hydrocarbyl group) may optionally be replaced by O, S, SO, SO₂, NR^{c}, X¹C(X²), C(X²)X¹ or X¹C(X²)X¹;
R^{c} is selected from R^{b}, hydrogen and C₁₋₄ hydrocarbyl; and
X¹ is O, S or NR^{c} and X² is ═O, ═S or NR^{c}.

Where the substituent group R¹⁰ comprises or includes a carbocyclic or heterocyclic group, the said carbocyclic or heterocyclic group may be unsubstituted or may itself be substituted with one or more further substituent groups independently selected from R¹⁰. In one sub-group of compounds of the formula (I), such further substituent groups R¹⁰ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R¹⁰.

The substituents R¹⁰ may be selected such that they contain no more than 20 non-hydrogen atoms, for example, no more than 15 non-hydrogen atoms, e.g. no more than 12, or 11, or 10, or 9, or 8, or 7, or 6, or 5 non-hydrogen atoms.

Where the carbocyclic and heterocyclic groups have a pair of substituents on the same or adjacent ring atoms, the two substituents may be linked so as to form a cyclic group. Thus, two adjacent groups R¹⁰, together with the carbon atoms or heteroatoms to which they are attached may form a 5-membered heteroaryl ring or a 5- or 6-membered non-aromatic carbocyclic or heterocyclic ring, wherein the said heteroaryl and heterocyclic groups contain up to 3 heteroatom ring members selected from N, O and S. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group.

Examples of such linked substituent groups include:

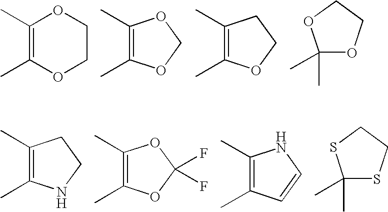

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone and consisting of carbon and hydrogen atoms, except where otherwise stated.

In certain cases, as defined herein, one or more of the carbon atoms making up the carbon backbone may be replaced by a specified atom or group of atoms.

Examples of hydrocarbyl groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or, where stated, substituted by one or more substituents as defined herein. The examples and preferences expressed below apply to each of the hydrocarbyl substituent groups or hydrocarbyl-containing substituent groups referred to in the various definitions of substituents for compounds of the formula (I) unless the context indicates otherwise.

The prefix “C_{x-y}” (where x and y are integers) as used herein refers to the number of carbon atoms in a given group. Thus, a C₁₋₄ hydrocarbyl group contains from 1 to 4 carbon atoms, and a C₃₋₆ cycloalkyl group contains from 3 to 6 carbon atoms, and so on.

The term “acyclic hydrocarbyl” (e.g. as in “acyclic C₁₋₅ hydrocarbyl”) as used herein refers to non-cyclic hydrocarbyl groups and in particular to alkyl, alkenyl and alkynyl groups as defined herein.

The term “mono- or di-C₁₋₅ hydrocarbylamino” as used herein refers to a monosubstituted or disubstituted amine group bearing either one or two hydrocarbyl substituent groups that each contain from 1 to 5 carbon atoms.

Preferred non-aromatic hydrocarbyl groups are saturated groups such as alkyl and cycloalkyl groups.

Generally by way of example, the hydrocarbyl groups can have up to ten carbon atoms (and more typically up to eight carbon atoms), unless the context requires otherwise. Within the sub-set of hydrocarbyl groups having 1 to 10 carbon atoms, particular examples are C₁₋₈ hydrocarbyl groups or C₁₋₆ hydrocarbyl groups, such as C₁₋₄ hydrocarbyl groups (e.g. C₁₋₃ hydrocarbyl groups or C₁₋₂ hydrocarbyl groups or C₂₋₃ hydrocarbyl groups or C₂₋₄ hydrocarbyl groups), specific examples being any individual value or combination of values selected from C₁, C₂, C₃, C₄, C₅, C₆, C₇, C₈, C₉ and C₁₀ hydrocarbyl groups.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups or C₂₋₃ alkyl groups or C₂₋₄ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 10 carbon atoms, more typically 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 10 carbon atoms, more typically 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 10 carbon atoms, more typically 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 10 carbon atoms, more typically 2 to 8 carbon atoms, particular examples are C₂₋₆ alkynyl groups, such as C₂₋₄ alkynyl groups.

Examples of carbocyclic aryl groups include substituted and unsubstituted phenyl groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

The terms C₁₋₁₂ hydrocarbyl, C₁₋₁₀ hydrocarbyl and C₁₋₈ hydrocarbyl as used herein encompass alkyl, alkenyl, alkynyl, cycloalkyl, cycloalkenyl, phenyl, benzyl and phenylethyl groups wherein the preferences for and examples of each of the aforesaid groups are as defined above. Within this definition, particular hydrocarbyl groups are alkyl, cycloalkyl, phenyl, benzyl and phenylethyl (e.g. 1-phenylethyl or 2-phenylethyl) groups, one subset of hydrocarbyl groups consisting of alkyl and cycloalkyl groups and in particular C₁₋₄ alkyl and cycloalkyl groups such as methyl, ethyl, n-propyl, isopropyl, n-butyl, isobutyl, tert-butyl, cyclopropyl and cyclobutyl.

The term C₁₋₄ hydrocarbyl as used herein encompasses alkyl, alkenyl, alkynyl, cycloalkyl and cycloalkenyl groups wherein the preferences for and examples of the aforesaid groups are as defined above. Within this definition, particular C₁₋₄ hydrocarbyl groups are alkyl and cycloalkyl groups, such as methyl, ethyl, n-propyl, isopropyl, n-butyl, isobutyl, tert-butyl, cyclopropyl and cyclobutyl.

When present, and where stated, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or trifluoromethyl. In one embodiment preferred substituents include monocyclic carbocyclic and heterocyclic groups having 3-7 ring members, more usually 3, 4, 5 or 6 ring members.

Where stated, one or more carbon atoms of a hydrocarbyl group may optionally be replaced by O, S, SO, SO₂, NR^{c}, X¹C(X²), C(X²)X¹ or X¹C(X²)X¹ (or a sub-group thereof) wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}). Further examples include ureas, carbonates and carbamates (C—C—C replaced by X¹C(X²)X¹).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members, more usually 5 to 6 ring members.

The term “aza-cycloalkyl” as used herein refers to a cycloalkyl group in which one of the carbon ring members has been replaced by a nitrogen atom. Thus examples of aza-cycloalkyl groups include piperidine and pyrrolidine. The term “oxa-cycloalkyl” as used herein refers to a cycloalkyl group in which one of the carbon ring members has been replaced by an oxygen atom. Thus examples of oxa-cycloalkyl groups include tetrahydrofuran and tetrahydropyran. In an analogous manner, the terms “diaza-cycloalkyl”, “dioxa-cycloalkyl” and “aza-oxa-cycloalkyl” refer respectively to cycloalkyl groups in which two carbon ring members have been replaced by two nitrogen atoms, or by two oxygen atoms, or by one nitrogen atom and one oxygen atom. Thus, in an oxa-C₄₋₆ cycloalkyl group, there will be from 3 to 5 carbon ring members and an oxygen ring member. For example, an oxa-cyclohexyl group is a tetrahydropyranyl group.

The definition “R^{a}-R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NC)NR^{c}, NR^{c}C(NR^{c}NR^{c}, S, SO, SO₂ NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₁₀ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkylalkoxy (e.g. C₃₋₆ cycloalkyl-C₁₋₂ alkoxy such as cyclopropylmethoxy). The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups may be substituted by a monocyclic group such as pyrrolidine, piperidine, morpholine and piperazine and N-substituted derivatives thereof such as N-benzyl, N—C₁₋₄ acyl and N—C₁₋₄ alkoxycarbonyl. Particular examples include pyrrolidinoethoxy, piperidinoethoxy and piperazinoethoxy.

When R^{a} is a bond and R^{b} is a C₁₋₁₀ hydrocarbyl group, examples of hydrocarbyl groups R^{a}-R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl and trifluoromethyl), or hydroxy (e.g. hydroxymethyl and hydroxyethyl), C₁₀ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}-R^{b} where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}-R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₁₀ hydrocarbyl group, or a carbocyclic or heterocyclic group. Examples of R^{a}-R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino, cyclopentylamino and cyclohexylamino).

### Specific Embodiments of and Preferences for R¹, R², R³, R¹⁰, R¹¹, R^{a}, R^{b}, R^{e}, R^{d}, R^{e}, X¹ and X²

### R¹

R¹ is a monocyclic or bicyclic carbocyclic or heterocyclic ring of 5 to 10 ring members of which up to two ring members may be heteroatoms selected from N, O and S and the remainder are carbon atoms, the carbocyclic or heterocyclic ring being optionally substituted by one or more substituent groups independently selected from R¹⁰.

Examples of monocyclic or bicyclic carbocyclic or heterocyclic rings are as set out in the General Preferences and Definitions section above. The rings can be unsubstituted or substituted by one or more substituent groups independently selected from R¹⁰ as defined herein.

Preferably, the carbocyclic or heterocyclic rings are substituted.

Preferably, the carbocyclic and heterocyclic rings are linked to the pyrimidine or triazine moiety via a carbon atom of the carbocyclic or heterocyclic rings.

The carbocyclic or heterocyclic rings can be monocyclic or bicyclic. Examples of such rings are 5-membered rings, 6-membered rings, 5.6-fused bicyclic rings and 6.6-fused bicyclic rings.

Preferably, the monocyclic and bicyclic rings are aryl or heteroaryl rings. An aryl or heteroaryl bicyclic ring may have two aromatic rings or one aromatic ring and one non-aromatic ring. In the latter case, it is preferred that the point of attachment of the group R¹ to the remainder of the molecule is on an aromatic ring.

In one embodiment, R¹ is a monocyclic ring and more particularly is a monocyclic aryl ring, i.e. a 6-membered carbocyclic aryl ring.

Particular examples of 6-membered rings are optionally substituted phenyl and pyridyl rings. The phenyl and pyridyl rings typically have up to 3 substituents selected from the group R¹⁰ as defined herein. Optionally substituted phenyl rings are particularly preferred.

In one sub-group of compounds, the monocyclic or bicyclic heterocyclic ring R¹ is unsubstituted or is substituted by one, two or three (preferably one or two) substituents selected from a group R^{10ao} consisting of halogen, hydroxy, amino and a group R^{a}-R^{b} where R^{a} is selected from a bond, O, CO, C(O)O, C(O)NR^{c}, NR^{c}C(O), NR^{c}C(O)O, NR^{c}, SQ, SO₂, SONR^{c}, and SO₂NR^{c}; and R^{b} is selected from hydrogen; carbocyclic and heterocyclic groups having 5 or 6 ring members; and C₁₋₁₀ hydrocarbyl (e.g. C₁₋₈ hydrocarbyl such as C₁₋₈ alkyl or C₃₋₇ cycloalkyl) optionally substituted by one or more substituents selected from hydroxy, oxo, cyano, amino, mono- or di-C₁₋₈ non-aromatic hydrocarbylamino, (e.g. mono- or di-C₁₋₄ hydrocarbylamino), carboxy, and carbocyclic and heterocyclic groups having from 3 to 7 ring members, and wherein one or more of the carbon atoms of the C₁₋₁₀ hydrocarbyl (e.g. C₁₋₈ hydrocarbyl such as C₁₋₈ alkyl or C₃₋₇ cycloalkyl) group may optionally be replaced by O, S, C(O)O, C(O)NR^{c} or NR^{c}.

In another sub-group of compounds, the monocyclic or bicyclic heterocyclic ring R¹ is unsubstituted or is substituted by one, two or three (preferably one or two) substituents selected from a group R^{10a} consisting of halogen, hydroxy, amino and a group R^{a}-R^{b} where R^{a} is selected from a bond, O, CO, C(O)O, C(O)NR^{c}, NR^{c}C(O), NR^{c}C(O)O, NR^{c}, SO, SO₂, SONR^{c}, and SO₂NR^{c}; and R^{b} is selected from hydrogen; carbocyclic and heterocyclic groups having 5 or 6 ring members; and C₁₋₁₀ hydrocarbyl (e.g. C₁₋₈ hydrocarbyl such as C₁₋₈ alkyl or C₃₋₇ cycloalkyl) optionally substituted by one or more substituents selected from hydroxy, oxo, amino, mono- or di-C₁₋₈ non-aromatic hydrocarbylamino, (e.g. mono- or di-C₁₋₄ hydrocarbylamino), carboxy, and carbocyclic and heterocyclic groups having from 3 to 7 ring members, and wherein one or more of the carbon atoms of the Cl₁₋₁₀ hydrocarbyl (e.g. C₁₋₈ hydrocarbyl such as C₁₋₈ alkyl or C₃₋₇ cycloalkyl) group may optionally be replaced by O, S, C(O)O, C(O)NR^{c} or NR^{c}.

Within this sub-group of compounds and sub-groups, preferences and examples thereof, where it is stated that one or more of the carbon atoms of the C₁₋₈ hydrocarbyl group may optionally be replaced by O, S, C(O)O, C(O)NR^{c} or NR^{c}, the orientation of the ester and amide groups may be in either direction unless indicated to the contrary.

In groups R¹⁰, R^{10a} and R^{10a}, the carbocyclic and heterocyclic groups having from 3 to 7 ring members and the carbocyclic and heterocyclic groups having 5 or 6 ring members, may each be unsubstituted or may bear one or more further substituents R^{10′}, R^{10ao′} or R₁₀ₐₐ respectively, wherein R^{10′}, R^{10ao′} and R₁₀ₐₐ correspond to R¹⁰, R^{10ao} and R^{10a} respectively except that they do not include a moiety containing a substituted carbocyclic group or substituted heterocyclic group.

For example, in the above sub-groups, when R^{b} is a carbocyclic or heterocyclic group, the carbocyclic or heterocyclic group may be substituted by one or more substituents R^{10′}, R^{10ao′} or R^{10aa} as defined herein. For example, when R^{b} is a carbocyclic or heterocyclic group, the carbocyclic or heterocyclic group may be substituted by one or more substituents selected from CO₂R¹⁴ wherein R¹⁴ is hydrogen or C₁₋₆ alkyl;

C₁₋₄ alkyl optionally substituted by hydroxy or C₁₋₂ alkoxy;
C₁₋₄ alkoxy optionally substituted by hydroxy or C₁₋₂ alkoxy; or
a group [sol], CH₂[sol], C(O)[sol], OCH₂CH₂[sol] or OCH₂CH₂CH₂[sol] where [sol] is as defined below.

In another sub-group, the carbocyclic or heterocyclic group R¹ is substituted by one, two or three (preferably one or two) substituents independently selected from a group R^{10b} consisting of halogen, OH, NH₂, CH₂OH, C₁₋₆ alkyl, CH₂NH₂, O—C₁₋₆-alkyl, aryl, heteroaryl, C₃₋₇ cycloalkyl, heterocyclyl, O-heteroaryl, O—C₃₋₇ cycloalkyl, O-heterocycloalkyl, C(═O)C₁₋₆ alkyl, C(═O)OC₁₋₆ alkyl, C(═O)NH₂, C(═O)NHC₁₋₆ alkyl, C(═O)N(C₁₋₆ alkyl)₂, NH(C₁₋₆ alkyl), N(C₁₋₆ alkyl)₂, NC(═O)C₁₋₆ alkyl, C₆ aryl, O—C₆ aryl, C(═O)C₆aryl, C(═O)OC₆aryl, C(═O)NH₂, C(═O)NHC₆aryl, NH(C₆ aryl), NC(═O)C₆ aryl, C₅₋₆ heterocyclyl, OC₅₋₆ heterocyclyl, C(═O)C₅₋₆ heterocyclyl, C(═O)OC₅₋₆ heterocyclyl, C(═O)NHC₅₋₆ heterocyclyl, C(═O)N(C₅₋₆ heterocyclyl)₂, NH(C₅₋₆ heterocyclyl), N(C₅₋₆ heterocyclyl)₂, NC(═O)C₅₋₆ heterocyclyl, C(═O)NHC₁₋₆ alkyl, C₅₋₆ aryl, S(═O)C₁₋₆ alkyl, S(═O)N—C₁₋₆ alkyl and SO₂N—C₁₋₆ alkyl; and a group [sol], CH₂[sol], CH₂CH₂[sol] or OCH₂CH₂[sol] where [sol] is selected from the following groups:

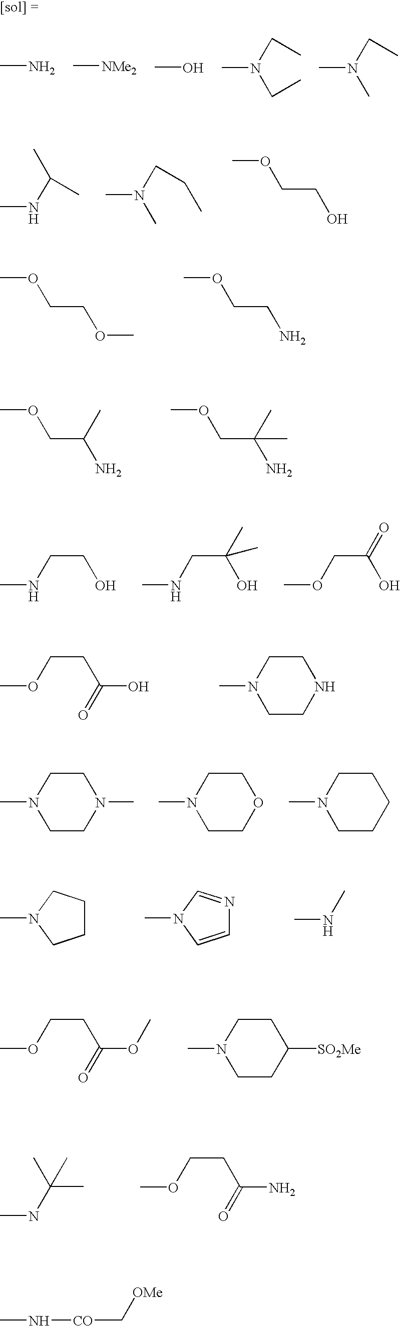

In another sub-group of compounds, the carbocyclic or heterocyclic ring R¹ is substituted by 1, 2 or 3 substituents R^{10c} is selected from:
hydroxy;
cyano;
halogen;
CO₂R¹⁴ wherein R¹⁴ is hydrogen or C₁₋₆ alkyl;
C₁₋₄ alkyl optionally substituted by hydroxy, fluorine, cyano or C₁₋₂ alkoxy;
C₁₋₄ alkoxy optionally substituted by hydroxy, fluorine, cyano or C₁₋₂ alkoxy; or a group [sol], CH₂[sol], C(O)[sol], OCH₂CH₂[sol] or OCH₂CH₂CH₂[sol] where [sol] is selected from: hydroxy, amino, C₁₋₄ alkylamino, di-C₁₋₄ alkylamino, 2-hydroxy-ethoxy, 2-methoxy-ethoxy, 2-amino-ethoxy, 2-amino-propoxy, 2-amino-2-methylpropoxy, 2-hydroxy-ethylamino, 2-hydroxy-propylamino, 2-hydroxy-2-methyl-propylamino, a group —O—(CH₂)ₚ(CR¹⁷R¹⁸)_{q}C(O)R¹⁶, 1-piperazino, 4-methyl-1-piperazino, 4-morpholino, 1-piperidino, 1-pyrrolidino, 1-imidazolyl, 4-methylsulphonyl-1-piperidino, 2-dimethylamino-1-hydroxyethyl, 1-dimethylamino-2-hydroxyethyl,

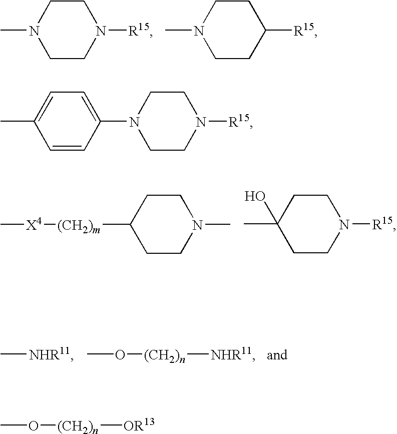

wherein p is 0, 1 or 2 and q is 0 or 1, provided that the sum of p and q is 1, 2 or 3:
R¹⁶ is OH, NH₂, NHMe or C₁₋₄ alkoxy;
R¹⁷ and R¹⁸ are each independently selected from hydrogen and methyl;
X⁴ is NH or O, m is 0 or 1, n is 1, 2 or 3;
R¹ is hydrogen, COR¹², C(O)OR¹² or R¹²;
R¹² is C₁₋₆ alkyl, C₃₋₆ cycloalkyl, aryl, aryl-C₁₋₆ alkyl or CH₂R¹⁵;
R¹⁵ is selected from hydrogen, C₁₋₆ alkyl, C₃₋₆ cycloalkyl, hydroxy-C₁₋₆ alkyl, piperidine, N—C₁₋₆ alkylpiperazine, piperazine, morpholine, COR¹³ and
C(O)OR¹³; and
   R¹³ is C₁₋₆ alkyl.

In another sub-group of compounds, the carbocyclic or heterocyclic ring R¹ is substituted by 1, 2 or 3 substituents R^{10cc} selected from:

halogen;
CO₂R¹⁴ wherein R¹⁴ is hydrogen or C₁₋₆ alkyl;
C₁₋₆ alkyl optionally substituted by hydroxy, cyano or C₁₋₂ alkoxy;
C₁₋₆ alkoxy optionally substituted by hydroxy, cyano or C₁₋₂ alkoxy; or a group [sol], CH₂[sol], C(O)[sol], OCH₂CH₂[sol] or OCH₂CH₂CH₂[sol] where [sol] is selected from: hydroxy, amino, C₁₋₄ alkylamino, di-C₁₋₄ alkylamino, 2-hydroxy-ethoxy, 2-methoxy-ethoxy, 2-amino-ethoxy, 2-amino-propoxy, 2-amino-2-methylpropoxy, 2-hydroxy-ethylamino, 2-hydroxy-propylamino, 2-hydroxy-2-methyl-propylamino, a group —O—(CH₂)ₚ(CR¹⁷R¹⁸)_{q}C(O)R¹⁶, 1-piperazino, 4-methyl-1-piperazino, 4-morpholino, 1-piperidino, 1-pyrrolidino, 1-imidazolyl, 4-methylsulphonyl-1-piperidino, 2-dimethylamino-1-hydroxyethyl, 1-dimethylamino-2-hydroxyethyl,

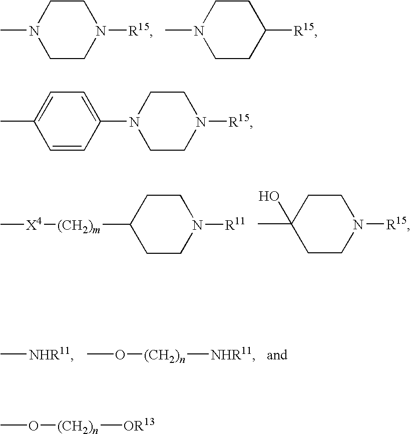

wherein p is 0, 1 or 2 and q is 0 or 1, provided that the sum of p and q is 1, 2 or 3:
R¹⁶ is OH; NH₂; NHMe; or C₁₋₄ alkoxy optionally substituted by a group R²¹ wherein R²¹ is selected from C₃₋₆ cycloalkyl, piperidine-4-yl, N—C₁₋₄ alkanoylpiperidin-4-yl, N—C₁₋₄alkoxycarbonyl-piperidin-4-yl, N—C₁₋₆ alkylpiperazine, piperazine, morpholine and tetrahydropyran;
R¹⁷ and R¹⁸ are each independently selected from hydrogen and methyl;

### X⁴ is NH or O, m is 0 or 1, n is 1, 2 or 3;

R¹¹ is hydrogen, COR¹², C(O)OR¹² or R¹²;
R¹² is C₁₋₆ alkyl, C₃₋₆ cycloalkyl, aryl, aryl-C₁₋₆ alkyl or CH₂R¹⁵;
R¹⁵ is selected from hydrogen, C₁₋₆ alkyl, C₃₋₆ cycloalkyl, hydroxy-C₁₋₆ alkyl, piperidine, N—C₁₋₆ alkylpiperazine, piperazine, morpholine, COR¹³ and C(O)OR¹³; and
R¹³ is C₁₋₆ alkyl.

More particular examples of substituents on the monocyclic and bicyclic rings are one or more substituents (e.g. 1, 2 or 3 substituents) independently selected from halogen (particularly fluorine and chlorine); amino; mono- and di-C₁₋₄alkylamino; optionally substituted (e.g. unsubstituted) monocyclic aryl and heteroaryl groups of 5 or 6 ring members and containing up to 1 heteroatom ring member; C₁₋₄ alkoxy (e.g. methoxy); C₁₋₄ alkyl; benzamido; and C₁₋₄ alkanoylamino; wherein the C₁₋₄ alkoxy, C₁₋₄ alkyl and C₁₋₄ alkanoylamino moieties are optionally substituted by hydroxy, halogen, C₁₋₂ alkoxy or a 5- or 6-membered saturated carbocyclic or heterocyclic group (e.g. piperidine, morpholine, pyrrolidine, piperazine and N—C₁₋₄ alkylpiperazine).

Where the carbocyclic and heterocyclic rings are monocyclic, it is preferred that the rings have a substituent on at least one of the ring atoms adjacent the point of attachment of the ring to the pyrimidine or triazine ring. Thus, for example, where the ring is a substituted phenyl ring, the phenyl ring preferably has a substituent on at least one of the 2- and 6-positions thereof. In one embodiment, the 2-position is substituted and the 6-position is unsubstituted, and in another embodiment, both the 2- and 6-positions are substituted. In a further embodiment, the 2- and 4-positions are both substituted. In other embodiments, the phenyl ring is trisubstituted with substituents at the 2- and 4-positions and at either of the 5 or 6-positions. In a still further embodiment, the phenyl ring is disubstituted with substituents at the 2- and 3-positions.

In one sub-group of compounds of the formula (I), R¹ is a phenyl group having substituents at the 2- and 4-positions thereof and optionally one or two further ring positions, wherein one of the 2- and 4-substituents is selected from R¹⁰ (and more preferably is selected from R^{10a0}, R^{10a}, R^{10b} R^{10c} or R^{10cc}) and the other of the 2- and 4-substituents is selected from halogen (particularly fluorine and chlorine); amino; mono- and di-C₁₋₄alkylamino; C₁₋₄ alkoxy (e.g. methoxy); C₁₋₄ alkyl; and C₁₋₄ alkanoylamino; wherein the C₁₋₄ alkoxy, C₁₋₄ alkyl and C₁₋₄ alkanoylamino moieties are optionally substituted by hydroxy, halogen or C₁₋₂ alkoxy.

In another sub-group of compounds of the formula (I), R¹ is a phenyl group having substituents at the 2- and 6-positions thereof and optionally one or two further ring positions, wherein one of the 2- and 6-substituents is selected from R¹⁰ (and more particularly is selected from R^{10a0}, R^{10a}, R^{10b} R^{10c} or R^{10cc}) and the other of the 2- and 6-substituents is selected from halogen (particularly fluorine and chlorine); amino; mono- and di-C₁₋₄alkylamino; C₁₋₄ alkoxy (e.g. methoxy); C₁₋₄ alkyl; and C₁₋₄ alkanoylamino; wherein the C₁₋₄ alkoxy, C₁₋₄ alkyl and C₁₋₄ alkanoylamino moieties are optionally substituted by hydroxy, halogen or C₁₋₂ alkoxy.

Particular examples of the group R¹ are set out in Table 1 below. In the table, the point of attachment of the group to the pyrimidine or triazine ring is indicated by means of an asterisk.

TABLE 1

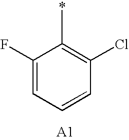

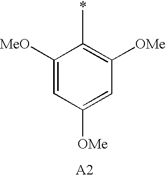

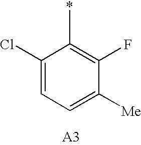

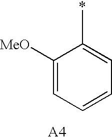

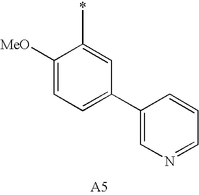

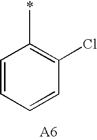

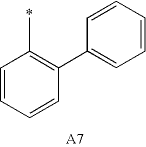

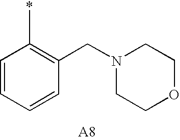

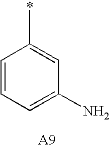

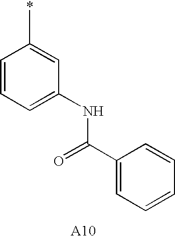

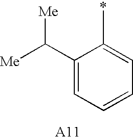

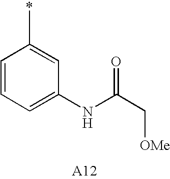

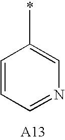

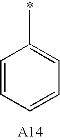

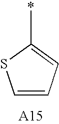

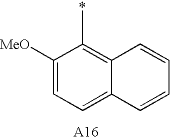

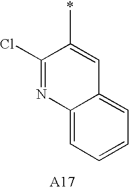

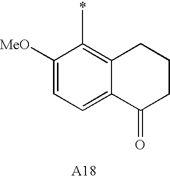

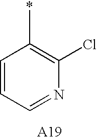

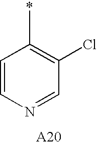

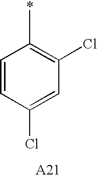

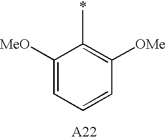

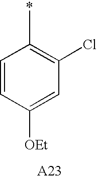

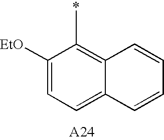

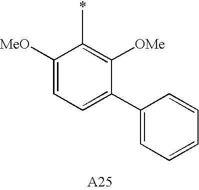

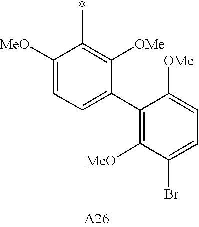

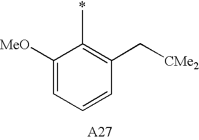

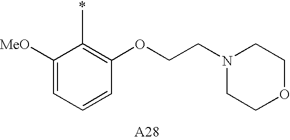

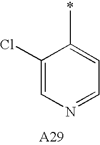

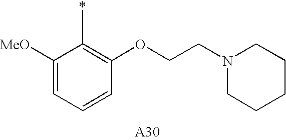

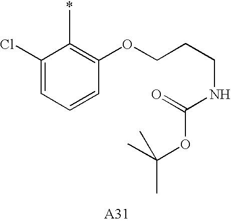

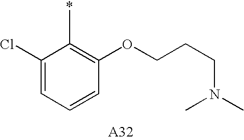

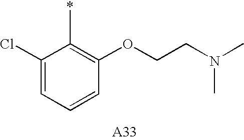

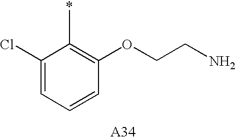

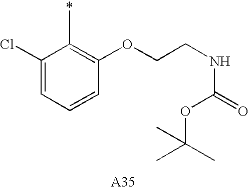

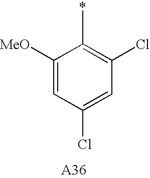

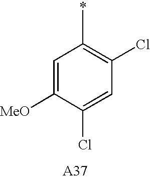

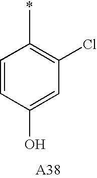

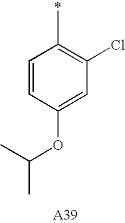

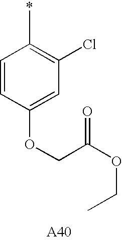

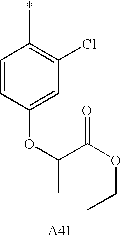

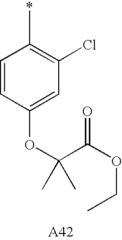

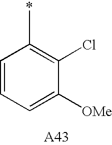

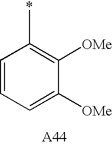

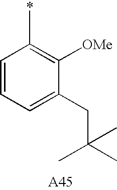

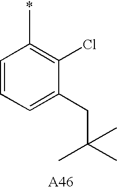

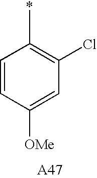

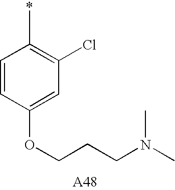

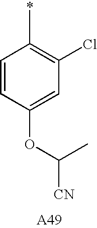

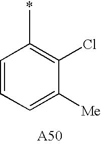

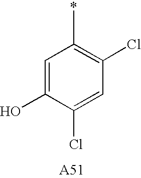

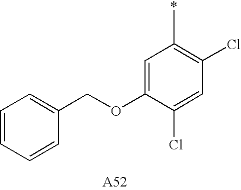

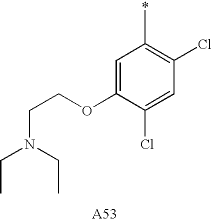

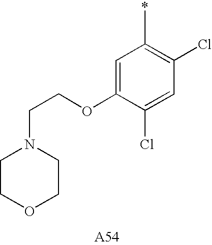

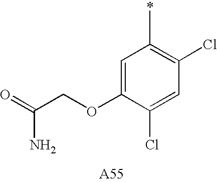

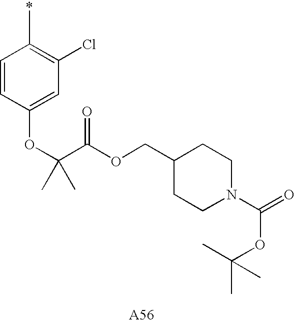

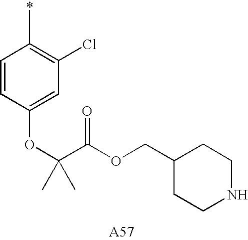

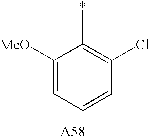

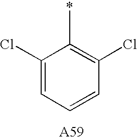

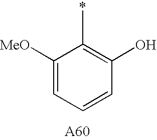

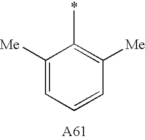

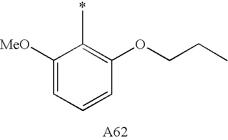

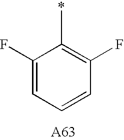

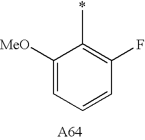

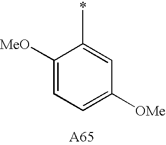

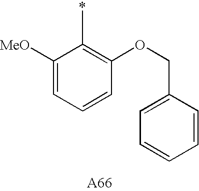

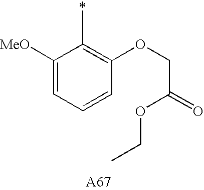

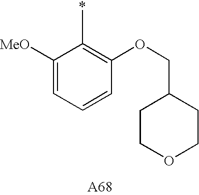

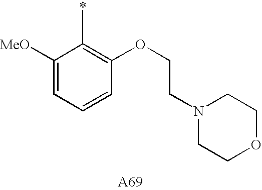

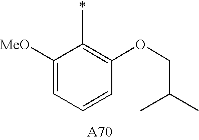

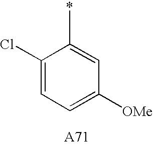

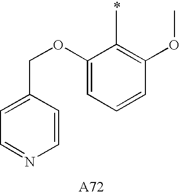

One sub-group of groups R¹ in Table 1 consists of groups A1 to A22.

Another sub-group of groups R¹ in Table 1 consists of groups A1 to A72.

One set of preferred groups R¹ in Table 1 consists of groups A2, A6, A21, A22 and A23.

Another set of preferred groups R¹ in Table 1 consists of groups A2, A6, A21, A22, A23, A35, A36, A37, A39, A40, A41, A42, A46, A47, A49, A51, A52, A53, A54, A55, A56 and A57.

### R²

R² is selected from:
hydrogen
halogen;
trifluoromethyl;
cyano;
amino;
mono- and di-C₁₋₄ hydrocarbylamino;
an acyclic C₁₋₁₀ hydrocarbyl group optionally substituted by one or more substituents independently selected from R¹¹ and wherein one or more carbon atoms of the acyclic C₁₋₁₀ hydrocarbyl group may optionally be replaced by O, S, SO, SO₂, NR^{c}, X¹C(X²), C(X²)X¹ or X¹C(X²)X¹;
   a group R^{d}-R^{e} wherein R^{d} is O, CO, X¹C(X²), C(X²)X¹, X¹C(X²)X¹, S, SO, SO₂, NR^{c}, SO₂NR^{c} or NR^{c}SO₂; and R^{e} is selected from hydrogen, carbocyclic and heterocyclic groups having from 3 to 12 ring members, and a C₁₋₁₀ hydrocarbyl group optionally substituted by one or more substituents independently selected from R¹¹, and wherein one or more carbon atoms of the C₁₋₁₀ hydrocarbyl group may optionally be replaced by O, S, SO, SO2, NR^{c}, X¹C(X²), C(X²)X¹ or X¹C(X²)X¹; provided that R^{d}-R^{e} is not hydroxy.

More particularly, R² is selected from:
hydrogen;
chlorine; fluorine; and bromine (preferably chlorine);
cyano;
amino;
mono- and di-C₁₋₄ hydrocarbylamino;
an acyclic C₁₋₈ hydrocarbyl group optionally substituted by one or more substituents independently selected from hydroxy, oxo, halogen, cyano, carboxy, amino, mono- or di-C₁₋₄ hydrocarbylamino, monocyclic carbocyclic and heterocyclic groups having from 3 to 7 ring members and wherein one or more carbon atoms of the acyclic C₁₋₈ hydrocarbyl group may optionally be replaced by O, S, SO, SO₂, NR^{c}, X¹C(X¹), C(X²)X¹ or X¹C(X¹)X¹;
   a group R^{d}-R^{e} wherein R^{d} is O, CO, X¹C(X²), C(X²)X¹, X¹C(X²)X¹, S, SO, SO₂, NR^{c}, SO₂NR^{c} or NR^{c}SO₂; and R^{e} is selected from hydrogen, monocyclic carbocyclic and heterocyclic groups having from 3 to 7 ring members, and an acyclic C₁₋₈ hydrocarbyl group optionally substituted by one or more substituents independently selected from hydroxy, oxo, halogen, cyano, carboxy, amino, mono- or di-C₁₋₄ hydrocarbylamino, carbocyclic and heterocyclic groups having from 3 to 7 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; provided that R^{d}-R^{e} is not hydroxy.

In one embodiment, R² is selected from:
hydrogen;
chlorine; fluorine; and bromine (preferably chlorine);
cyano;
amino;
mono- and di-C₁₋₄ hydrocarbylamino; and
   an acyclic C₁₋₈ hydrocarbyl group optionally substituted by one or more substituents independently selected from hydroxy, oxo, halogen, cyano, carboxy, amino, mono- or di-C₁₋₄ hydrocarbylamino, imidazolyl, and non-aromatic monocyclic carbocyclic and heterocyclic groups having from 3 to 7 ring members and wherein one or more carbon atoms of the acyclic C₁₋₈ hydrocarbyl group may optionally be replaced by O, S, SO, SO₂, NR^{c}, X¹C(X²), C(X²)X¹ or X¹C(X²)X¹.

Within this embodiment, more particular compounds are those wherein R² is selected from:
hydrogen;
chlorine; fluorine; and bromine (preferably chlorine);
cyano;
amino;
mono- and di-C₁₋₄ hydrocarbylamino; and
CH₂[sol] or OCH₂CH₂[sol] where [sol] is as defined herein.

Another subgroup within R² consists of hydrogen; amino; halogen; C₁₋₄ alkoxy; C₁₋₄ alkyl optionally substituted by fluorine, C₁₋₂ alkoxy, carbamoyl, N—C₁₋₂alkylcarbamoyl or C₁₋₂ alkoxycarbonyl; and C₁₋₄ alkylthio optionally substituted by C₁₋₂ alkoxy, carbamoyl, N—C₁₋₂ alkylcarbamoyl or C₁₋₂ alkoxycarbonyl.

Particular examples of R² are hydrogen, C₁₋₄ alkyl (e.g. methyl) and halogen (e.g. chlorine), with halogen (e.g. chlorine) being particularly preferred when A is a group CR³.

When A is N, R² is preferably other than halogen.

### A

A is selected from N and CR³.

When A is N, it is preferred that R² is other than halogen; i.e. the compound is other than a compound of the formula:

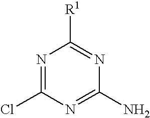

In one embodiment, A is N.

In another embodiment, A is CR³.

When A is CR³, R³ is selected from R² and monocyclic carbocyclic and heterocyclic groups having 3 to 7 ring members, wherein the monocyclic carbocyclic and heterocyclic groups are optionally substituted by one or more substituent groups independently selected from R¹⁰.

Particular examples and preferences for R² are as set out above and apply also to R³.

In one embodiment, R³ is selected from:
hydrogen;
hydroxy;
chlorine; fluorine; bromine;
cyano;
amino;
mono- and di-C₁₋₄ hydrocarbylamino;
a group [sol], CH₂[sol] or OCH₂CH₂[sol] where [sol] is as defined herein; and monocyclic non-aromatic carbocyclic and heterocyclic groups having 3 to 7 ring members and containing up to 2 heteroatom ring members selected from O, N and S (and S-oxides and S,S-dioxides thereof), wherein the non-aromatic monocyclic carbocyclic and heterocyclic groups are optionally substituted by one or more substituent groups independently selected from R¹⁰ as defined herein.

In one preferred embodiment, R³ is hydrogen.

Particular examples of the moiety:

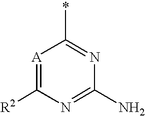

are set out in Table 2. The point of attachment to the monocylic or bicyclic carbocyclic or heterocyclic group R¹ is indicated by means of an asterisk.

TABLE 2

| | |
|---|---|
| 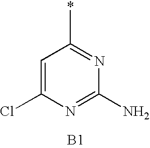 | 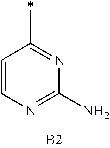 |
| 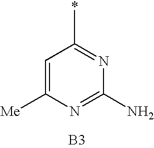 | 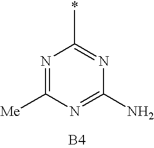 |
| 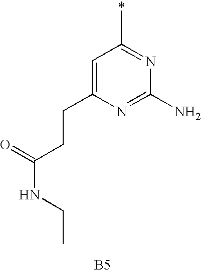 | 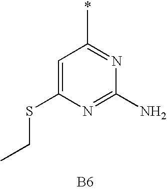 |
| 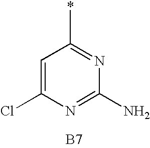 | 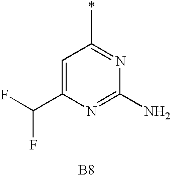 |
| 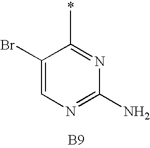 | 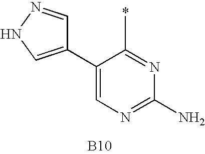 |
| 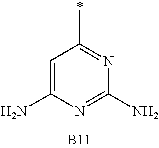 | 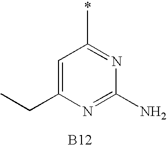 |
| 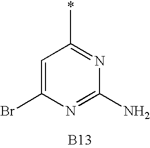 | 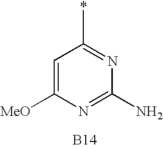 |
| 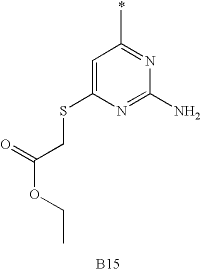 | 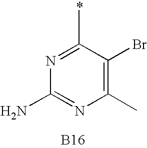 |

One set of groups consists of groups B1 to B4 and B7 in Table 1.

Another set of groups consists of groups B1 to B16 in Table 1.

Preferred groups are groups B1, B3 and B4.

### Particular and Preferred Sub-Groups of the Formula (I)

In this section, references to the groups R², R³, R⁴, R⁵, R⁶, R¹⁰, R^{10a0}, R^{10a}, R^{10b}, R^{10c} and R_{10cc} include references also to each of the sub-groups, preferences and examples thereof as defined in the preceding sections of this application, unless the context indicates otherwise.

One sub-group of compounds of the invention can be represented by the general formula (II):

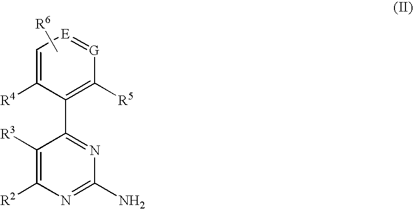

or salts, tautomers, solvates and N-oxides thereof; wherein:
E and G are each selected from nitrogen and CR⁶;
R² and R³ are as defined herein; and
R⁴, R⁵ and R⁶ are the same or different and are each hydrogen or a substituent R¹⁰, R^{10a0}, R^{10a}, R^{10b} R^{10c} or R^{10cc} as defined herein.

In one sub-group of compounds, one of R⁴ and R⁵ is hydrogen and the other is a substituent R¹⁰, R^{10a0}, R^{10a}, R^{10b} R^{10c} or R^{10cc} as defined herein. Within this sub-group, one particular group of compounds is the group of compounds wherein E and G are both CH and R⁶ is hydrogen, i.e. the ring is a 2-monosubstituted phenyl ring.

In another sub-group of compounds, R⁴ and R⁵ are each a substituent R¹⁰, R^{10a0}, R^{10a}, R^{10b} R^{10c} or R^{10cc} as defined herein. Within this sub-group, particular compounds are those wherein E and G are both CH and R⁶ is hydrogen or a substituent R¹⁰, R^{10a0}, R^{10a}, R^{10b} R^{10c} or R^{10cc}, i.e. the ring is a 2,6-disubstituted phenyl ring or a trisubstituted phenyl ring.

In another sub-group of compounds, E is CR^{10a}, G is CH, and R⁵ and R⁶ are both hydrogen, i.e. the ring is a 2,4-disubstituted phenyl ring.

In a further sub-group of compounds, R⁴, E and G are each independently selected from CR^{10a}, R⁵ is hydrogen, i.e. the ring is a 2,4,5-trisubstituted phenyl ring.

In formula (II), when R⁴ and R⁵ are each a substituent, one substituent may be selected from the full range of substituents R¹⁰, R^{10a0}, R^{10a}, R^{10b} R^{10c} or R^{10cc} as defined herein and the other may be the same or different and may be selected from a group of smaller substituents, for example a substituent selected from the group R^{10c} consisting of fluoro, chloro, bromo, trifluoromethyl, difluoromethoxy, trifluoromethoxy, cyano, methyl, ethyl, cyclopropyl, hydroxy, methylsulphonyl, amino, methylamino, dimethylamino, methoxy, ethoxy, hydroxymethyl, hydroxyethyl, ethoxycarbonyl, methoxycarbonyl, aminocarbonyl, methoxymethyl, carboxy, C₁₋₂ alkoxycarbonyl, aminocarbonyl, acetyl and methylsulphonyl. Within group R^{10c}, one sub-set of substituents consists of methyl, ethyl, chloro, fluoro, hydroxy, methylsulphonyl, amino, methylamino, dimethylamino, cyano, methoxy, ethoxy, hydroxymethyl, cyclopropyl, hydroxyethyl, ethoxycarbonyl, methoxycarbonyl, aminocarbonyl, oxo, methoxymethyl and acetyl.

In another embodiment, both R⁴ and R⁵ may be substituents selected from R^{10c} and sub-sets thereof as defined herein.

A further sub-group of compounds of the invention can be represented by the general formula (IIa):

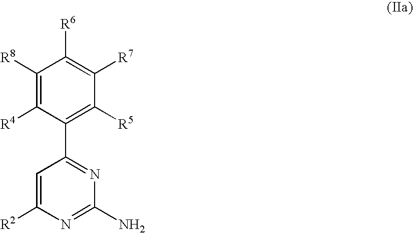

or salts, tautomers, solvates or N-oxides thereof; wherein:
(A) R² and R⁴ each are chlorine, and R⁵ to R⁸ each are hydrogen; or
(B) R² is C₁₋₆ alkylthio; R⁴ and R⁵ each are methoxy; and R⁶, R⁷ and R⁸ each are hydrogen; or
(C)R² is chlorine; R⁴ is chlorine; R⁶ is hydrogen or chlorine; R⁷ and R⁸ each are hydrogen; and R⁵ is a group R^{10cc} where R^{10cc} is selected from:
halogen;
CO₂R¹⁴ wherein R¹⁴ is hydrogen or C₁₋₆ alkyl;
C₁₋₆ alkyl optionally substituted by hydroxy, cyano or C₁₋₂ alkoxy;
C₁₋₆ alkoxy optionally substituted by hydroxy, cyano or C₁₋₂ alkoxy; or
a group [sol], CH₂[sol], C(O)[sol], OCH₂CH₂[sol] or OCH₂CH₂CH₂[sol] where [sol] is selected from: hydroxy, amino, C₁₋₄ alkylamino, di-C₁₋₄ alkylamino, 2-hydroxy-ethoxy, 2-methoxy-ethoxy, 2-amino-ethoxy, 2-amino-propoxy, 2-amino-2-methylpropoxy, 2-hydroxy-ethylamino, 2-hydroxy-propylamino, 2-hydroxy-2-methyl-propyl amino, a group —O—(CH₂)ₚ(CR¹⁷R¹⁸)_{q}C(O)R¹⁶, 1-piperazino, 4-methyl-1-piperazino, 4-morpholino, 1-piperidino, 1-pyrrolidino, 1-imidazolyl, 4-methylsulphonyl-1-piperidino, 2-dimethylamino-1-hydroxyethyl, 1-dimethylamino-2-hydroxyethyl,

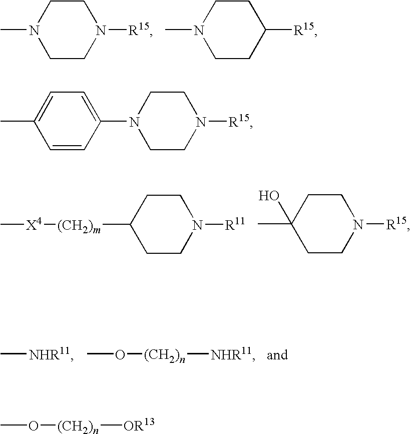

wherein p is 0, 1 or 2 and q is 0 or 1, provided that the sum of p and q is 1, 2 or 3:
R¹⁶ is OH; NH2; NHMe; or C₁₋₄ alkoxy optionally substituted by a group R²¹ wherein R²¹ is selected from C₃₋₆ cycloalkyl, piperidine-4-yl, N—C₁₋₄ alkanoylpiperidin-4-yl, N—C₁₋₄alkoxycarbonyl-piperidin-4-yl, N—C₁₋₆ alkylpiperazine, piperazine, morpholine and tetrahydropyran;
   R¹⁷ and R¹⁸ are each independently selected from hydrogen and methyl;
X⁴ is NH or O, m is 0 or 1, n is 1, 2 or 3;
R¹¹ is hydrogen, COR¹², C(O)OR¹² or R¹²;
R¹² is C₁₋₆ alkyl, C₃₋₆ cycloalkyl, aryl, aryl-C₁₋₆ alkyl or CH₂R¹⁵;
R¹⁵ is selected from hydrogen, C₁₋₆ alkyl, C₃₋₆ cycloalkyl, hydroxy-C₁₋₆ alkyl, piperidine, N—C₁₋₆ alkylpiperazine, piperazine, morpholine, COR¹³ and
C(O)OR¹³; and
R¹³ is C₁₋₆ alkyl; or
   (D) R² is chlorine; R⁴ is chlorine; R⁵, R⁷ and R⁸ each are hydrogen; and R⁶ is a group R^{10cc}; or
   (E) R² is chlorine; R⁴ is chlorine or methoxy; R⁶ is chlorine; R⁵ and R⁸ each are hydrogen; and R⁷ is a group R^{10cc}; or
   (F) R² is chlorine; R⁴ is chlorine; R⁵, R⁶ and R⁷ each are hydrogen; and R⁸ is a group R^{10cc}.

In one embodiment (Embodiment (A)) of formula (IIa), R² and R⁴ each are chlorine, and R⁵ to R⁸ each are hydrogen.

In another embodiment (Embodiment (B)) of formula (IIa), R² is C₁₋₆ alkylthio; R⁴ and

R⁵ each are methoxy; and R⁶, R⁷ and R⁸ each are hydrogen.

In a further embodiment (Embodiment (C)) of formula (IIa), R² is chlorine; R⁴ is chlorine; R⁶ is hydrogen or chlorine; R⁷ and R⁵ each are hydrogen; and R⁵ is a group R^{10cc}.

In one sub-group of compounds within Embodiment (C), R⁶ is hydrogen and in another sub-group of compounds, R⁶ is chlorine.

Particular compounds within Embodiment (C) are those wherein R^{10cc} is:

fluorine;
chlorine;
C₁₋₆ alkyl optionally substituted by hydroxy, cyano or C₁₋₂ alkoxy;
C₁₋₆ alkoxy optionally substituted by hydroxy, cyano or C₁₋₂ alkoxy; or
a group [sol], CH₂[sol], OCH₂CH₂[sol] or OCH₂CH₂CH₂[sol] where [sol] is selected from: hydroxy, amino, C₁₋₄ alkylamino, di-C₁₋₄ alkylamino, 2-hydroxy-ethoxy, 2-methoxy-ethoxy, a group —O—(CH₂)ₚ(CR¹⁷R¹⁸)_{q}C(O)R¹⁶, 1-piperazino, 4-methyl-1-piperazino, 4-morpholino, 1-piperidino, 1-pyrrolidino, 1-imidazolyl and 4-methylsulphonyl-1-piperidino and NHR¹¹;
wherein p is 0, 1 or 2 and q is 0 or 1, provided that the sum of p and q is 1, 2 or 3;
R¹⁶ is OH, NH₂, NHMe or C₁₋₄ alkoxy;
R¹¹ is hydrogen, COR¹², C(O)OR¹² or R¹²;
R¹² is C₁₋₆ alkyl or CH₂R¹⁵;
R¹⁵ is selected from hydrogen, C₁₋₆ alkyl, hydroxy-C₁₋₆ alkyl, piperidine, N—C₁₋₄ alkylpiperazine, piperazine, morpholine, COR¹³ and C(O)OR¹³; and
R¹³ is C₁₋₄ alkyl.

More particular compounds within Embodiment (C) are compounds wherein the moiety

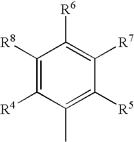

is any one of groups A1, A31, A32, A33, A34, A35, A36, A58 and A59 as set forth in Table 1 herein,

In another embodiment (Embodiment (D)) of formula (IIa), R² is chlorine; R⁴ is chlorine; R⁵, R⁷ and R⁸ each are hydrogen; and R⁶ is a group R^{10cc}.

Within Embodiment (D), one sub-group of compounds is the group in which R^{10cc} is:
chlorine;
C₁₋₆ alkyl optionally substituted by hydroxy, cyano or C₁₋₂ alkoxy;
C₁₋₆ alkoxy optionally substituted by hydroxy, cyano or C₁₋₂ alkoxy; or
a group [sol], CH₂[sol], C(O)[sol], OCH₂CH₂[sol] or OCH₂CH₂CH₂[sol] where [sol] is selected from: hydroxy, amino, C₁₋₄ alkylamino, di-C₁₋₄ alkylamino, 2-hydroxy-ethoxy, 2-methoxy-ethoxy, 2-amino-ethoxy, 2-amino-propoxy, 2-amino-2-methylpropoxy, 2-hydroxy-ethylamino, 2-hydroxy-propylamino, 2-hydroxy-2-methyl-propylamino, a group —O—(CH₂)ₚ(CR¹⁷R¹⁸)_{q}C(O)R¹⁶, 1-piperazino, 4-methyl-1-piperazino, 4-morpholino, 1-piperidino, 1-pyrrolidino, 1-imidazolyl, 4-methylsulphonyl-1-piperidino, 2-dimethylamino-1-hydroxyethyl, 1-dimethylamino-2-hydroxyethyl, NHR¹¹, and —O—(CH₂)ₙ—OR¹³
wherein p is 0, 1 or 2 and q is 0 or 1, provided that the sum of p and q is 1, 2 or 3:
R¹⁶ is OH; NH₂; NHMe; or C₁₋₄ alkoxy optionally substituted by a group R²¹ wherein
R²¹ is selected from C₃₋₆ cycloalkyl, piperidine-4-yl, N—C₁₋₄ alkanoylpiperidin-4-yl, N—C₁₋₄alkoxycarbonyl-piperidin-4-yl, N—C₁₋₆ alkylpiperazine, piperazine, morpholine and tetrahydropyran;
R¹⁷ and R¹⁸ are each independently selected from hydrogen and methyl;

### X⁴ is NH or O, m is 0 or 1, n is 1, 2 or 3;

R¹¹ is hydrogen, COR¹², C(O)OR¹² or R¹²;
R¹² is C₁₋₆ alkyl, C₃₋₆ cycloalkyl, or CH₂R¹⁵;
R¹⁵ is selected from hydrogen, C₁₋₆ alkyl, C₃₋₆ cycloalkyl, hydroxy-C₁₋₆ alkyl, piperidine, N—C₁₋₆ alkylpiperazine, piperazine, morpholine, COR¹³ and C(O)OR¹³; and
R¹³ is C₁₋₆ alkyl.

Within this sub-group, particular compounds are those wherein R^{10cc} is hydroxy; chlorine; C₁₋₄ alkoxy; or a group —O—(CH₂)ₚ(CR¹⁷R¹⁸)_{q}C(O)R¹⁶.

More particularly R^{10cc} is hydroxy, methoxy, ethoxy, isopropoxy, chlorine, or —O—(CH₂)ₚ(CR¹⁷R¹⁸)_{q}C(O)R¹⁶ where R¹⁶ is selected from NH₂ and C₁₋₂ alkoxy optionally substituted by a piperidine-4-yl or N—C₁₋₄ alkanoylpiperidin-4-yl group.

In one set of compounds within Embodiment (D), the moiety

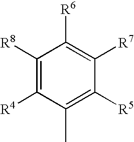

is any one of groups A21, A23, A38, A39, A40, A41, A42, A47, A48, A49, A56 and A57 in Table 1.

In another embodiment (Embodiment (E)) of formula (IIa), R² is chlorine; R⁴ is chlorine or methoxy; R⁶ is chlorine; R⁵ and R⁸ each are hydrogen; and R⁷ is a group R^{10cc}.

Preferably R⁴ is chlorine.

In one group of compounds within Embodiment (E), R^{10cc} is chlorine;

C₁₋₆ alkyl optionally substituted by hydroxy, cyano or C₁₋₂ alkoxy;
C₁₋₆ alkoxy optionally substituted by hydroxy, cyano or C₁₋₂ alkoxy; or
a group [sol], CH₂[sol], C(O)[sol], OCH₂CH₂[sol] or OCH₂CH₂CH₂[sol] where [sol] is selected from: hydroxy, amino, C₁₋₄ alkylamino, di-C₁₋₄ alkylamino, 2-hydroxy-ethoxy, 2-methoxy-ethoxy, 2-amino-ethoxy, 2-amino-propoxy, 2-amino-2-methylpropoxy, 2-hydroxy-ethylamino, 2-hydroxy-propylamino, 2-hydroxy-2-methyl-propylamino, a group —O—(CH₂)ₚ(CR¹⁷R¹⁸)_{q}C(O)R¹⁶, 1-piperazino, 4-methyl-1-piperazino, 4-morpholino, 1-piperidino, 1-pyrrolidino, 1-imidazolyl, 4-methylsulphonyl-1-piperidino, 2-dimethylamino-1-hydroxyethyl, 1-dimethylamino-2-hydroxyethyl, NHR¹¹, and —O—(CH₂)ₙ—OR¹³
wherein p is 0, 1 or 2 and q is 0 or 1, provided that the sum of p and q is 1, 2 or 3:
R¹⁶ is OH; NH₂; NHMe; or C₁₋₄ alkoxy optionally substituted by a group R²¹ wherein
R²¹ is selected from C₃₋₆ cycloalkyl, piperidine-4-yl, N—C₁₋₄ alkanoylpiperidin-4-yl, N—C₁₋₄alkoxycarbonyl-piperidin-4-yl, N—C₁₋₆ alkylpiperazine, piperazine, morpholine and tetrahydropyran;
R¹⁷ and R¹⁸ are each independently selected from hydrogen and methyl;

### X⁴ is NH or O, m is 0 or 1, n is 1, 2 or 3;

R¹¹ is hydrogen, COR¹², C(O)OR¹² or R¹²;
R¹² is C₁₋₆ alkyl, C₃₋₆ cycloalkyl, or CH₂R¹⁵;
R¹⁵ is selected from hydrogen, C₁₋₆ alkyl, C₃₋₆ cycloalkyl, hydroxy-C₁₋₆ alkyl, piperidine, N—C₁₋₆ alkylpiperazine, piperazine, morpholine, COR¹³ and C(O)OR¹³; and
R¹³ is C₁₋₆ alkyl.

Within this group of compounds, one sub-set consists of the compounds wherein R^{10cc} is hydroxy; chlorine; C₁₋₄ alkoxy; or a group —O—(CH₂)ₚ(CR¹⁷R¹⁸)_{q}C(O)R¹⁶.

Particular compounds within Embodiment (E) are those wherein the moiety

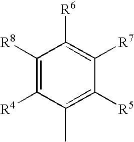

is any one of groups A37, A51, A52, A53, A54 and A55 in Table 1.

In another embodiment (Embodiment (F)) of formula (IIa), R² is chlorine; R⁴ is chlorine; R⁵, R⁶ and R⁷ each are hydrogen; and R⁸ is a group R_{10cc}.

Within Embodiment (F), particular compounds are those wherein R⁸ is C₁₋₆ alkoxy or C₁₋₆ alkyl, and more particularly wherein R⁸ is methoxy or C₁₋₅ alkyl.

Examples of compounds within Embodiment (F) are those wherein the moiety

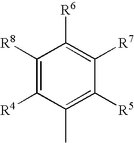

is any one of groups A43, A46 and A50 in Table 1 herein.

Another sub-group of compounds of the invention can be represented by the general formula (III):

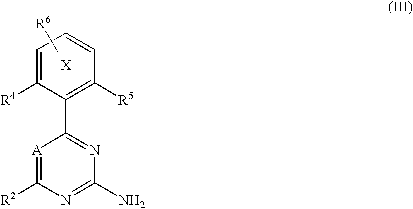

or salts, tautomers, solvates and N-oxides thereof; wherein:
ring X is a phenyl or pyridyl ring;

### A is CH or N;

R² is as defined herein; and
R⁴, R⁵ and R⁶ are each independently selected from R¹⁰, R^{10a0}, R^{10a}, R^{10b} R^{10c} or R^{10cc} as defined herein.

In one preferred group of compounds of the invention, A is CR³.

The ring X is preferably a phenyl ring.

Another sub-group of compounds of the invention can be represented by the general formula (IV):

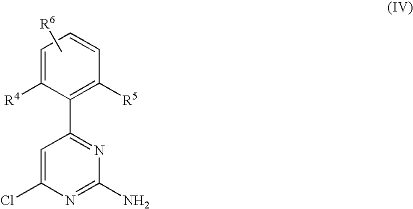

or salts, tautomers, solvates and N-oxides thereof; wherein:
R⁴, R⁵ and R⁶ are each independently selected from R¹⁰, R^{10a0}, R^{10a}, R^{10b} R^{10c} or R^{10cc} as defined herein

Within this sub-group, the phenyl ring can be, for example, 2-monosubstituted, 3-monosubstituted, 4-monosubstituted, 2,4-disubstituted, 2,5-disubstituted, 2,6-disubstituted, 2,3,6-trisubstituted and 2,4,6-trisubstituted.

In one embodiment, R⁶ is located para with respect to the pyrimidine ring.

Particular examples of R⁶ are hydrogen, methoxy and chlorine.

Within formula (IV), one group of compounds can be represented by the formula (IVa):

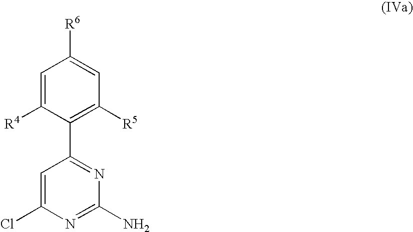

or salts, tautomers, solvates and N-oxides thereof; wherein:
R⁴ is a substituent R^{10a0}, R^{10a}, R^{10b} R^{10c} or R^{10cc};
R⁵ is a substituent R^{10a0}, R^{10a}, R^{10b} R^{10c} or R^{10cc};

R⁶ is hydrogen or a substituent R^{10a0}, R^{10a}, R^{10b} R^{10c} or R^{10cc}.

In one sub-set of compounds within formula (IVa), R⁶ is hydrogen.

A further sub-group of compounds of the invention can be represented by the general formula (V):

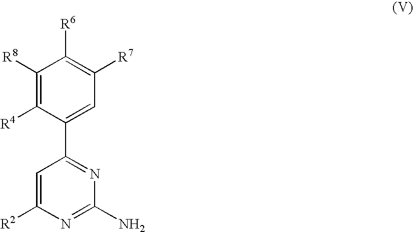

or salts, tautomers, solvates and N-oxides thereof; wherein:
R² is as defined herein;
R⁴ is a substituent R^{10a0}, R^{10a}, R^{10b} R^{10c} or R^{10cc};

R⁶ is hydrogen or a substituent R^{10a}, R^{10a}, R^{10b} R^{10c} or R^{10cc};

R⁷ is hydrogen or a substituent R^{10a}, R^{10a}, R^{10b} R^{10c} or R^{10cc}; and
R⁸ is hydrogen or a substituent R^{10a0}, R^{10a}, R^{10b} R^{10c} or R^{10cc};
provided that no more than two of R⁶, R⁷ and R⁸ are other than hydrogen.

Formula (V) embraces compounds in which the phenyl group is inter alia:

(a) 2-monosubstituted;
(b) 2,4-disubstituted;
(c) 2,5-disubstituted;
(d) 2,4,5-trisubstituted;
(e) 2,3-disubstituted; or
(f) 2,3,5-trisubsubstituted.

Each of groups (a), (b), (c), (d), (e) and (f) represents an embodiment of the invention.

Within formula (V), one sub-group of compounds can be represented by the general formula (VI):

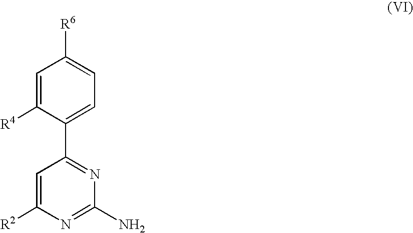

or salts, tautomers, solvates and N-oxides thereof; wherein:
R² is as defined herein;
R⁴ is a substituent R^{10a0}, R^{10a}, R^{10b} R^{10c} or R^{10cc}; and
R⁶ is a substituent R^{10a0}, R^{10a}, R^{10b} R^{10c} or R^{10cc}.

Within formula (VI), in one embodiment:

(i) R⁴ is selected from C₁₋₆ alkyl, C₁₋₆ alkoxy and halogen; and/or
(ii) R⁶ is selected from C₁₋₆ alkyl, C₁₋₆ alkoxy and halogen.

Within this sub-group of compounds, R⁴ may be selected from, for example, hydrogen C₁₋₆ alkyl, C₁₋₆ alkoxy and halogen. Particular examples of R⁴ are hydrogen, methyl, methoxy and chlorine.

In another embodiment within formula (VI):

R⁴ is selected from C₁₋₆ alkyl, C₁₋₆ alkoxy and halogen; and
R⁶ is selected from:
hydroxy;
C₁₋₆ alkyl;
halogen; and
C₁₋₆ alkoxy optionally substituted by:
   hydroxy,
   cyano,
   C₁₋₂ alkoxy,
   C₁₋₄-alkoxycarbonyl optionally substituted by a saturated 5- or 6-membered heterocyclic ring selected from pyrrolidino, piperidine, piperazino and morpholino, and wherein the heterocyclic ring is optionally further substituted by C₁₋₄ alkyl, C₁₋₄ acyl or C₁₋₄ alkoxycarbonyl;
   amino, or
   mono- or di-C₁₋₄-alkylamino, provided that when the optional substituent is hydroxy, C₁₋₂ alkoxy, amino, mono- or di-C₁₋₄-alkylamino, there are at least two carbon atoms between the oxygen atom of the optionally substituted alkoxy group and the optional substituent.

Another sub-group of compounds within formula (V) can be represented by the general formula (VII):

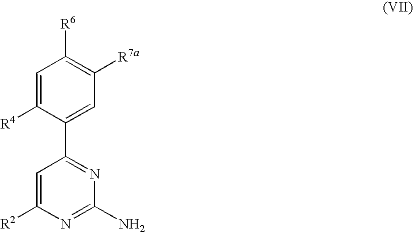

or salts, tautomers, solvates and N-oxides thereof; wherein:
R² is as defined herein;
R⁴ is a substituent R^{10a0}, R^{10a}, R^{10b} R^{10c} or R^{10cc};
R⁶ is a substituent R^{10a0}, R^{10a}, R^{10b} R^{10c} or R^{10cc}; and
R⁷ is a substituent R^{10a0}, R^{10a}, R^{10b} R^{10c} or R^{10cc}.

In one embodiment within formula (VII), R⁴ and R⁶ are each independently selected from C₁₋₆ alkyl; C₁₋₆ alkoxy; and halogen; and R⁷ is selected from hydroxy; C₁₋₆ alkyl; halogen; and optionally substituted C₁₋₆ alkoxy; wherein the optional substituent for C₁₋₆ alkoxy is:
hydroxy,
phenyl,
cyano,
C₁₋₂ alkoxy,
carbamoyl, or mono- or di-N—C₁₋₄ alkyl carbamoyl, or
C₁₋₄-alkoxycarbonyl optionally substituted by a saturated 5- or 6-membered heterocyclic ring selected from pyrrolidino, piperidine, piperazino and morpholino, and wherein the heterocyclic ring is optionally further substituted by C₁₋₄ alkyl, C₁₋₄ acyl or C₁₋₄ alkoxycarbonyl;
amino, or
mono- or di-C₁₋₄-alkylamino,
   provided that when the optional substituent for optionally substituted C₁₋₆ alkoxy is hydroxy, C₁₋₂ alkoxy, amino, mono- or di-C₁₋₄-alkylamino, there are at least two carbon atoms between the oxygen atom of the optionally substituted alkoxy group and the optional substituent.

In the above embodiment of formula (VII), it is preferred that R⁴ is chlorine and/or R⁶ is chlorine.

In a further embodiment within formula (V), the compounds can be represented by the formula (VIII):

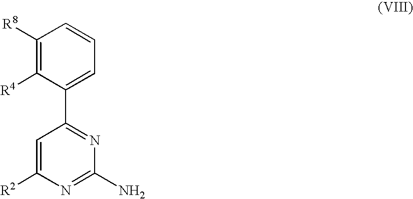

or salts, tautomers, solvates and N-oxides thereof; wherein:
R² is as defined herein;
R⁴ is a substituent R^{10a0}, R^{10a}, R^{10b} R^{10c} or R^{10cc}; and

R⁸ is a substituent R^{10a0}, R^{10a}, R^{10b} R^{10c} or R^{10cc}.

Within formula (VIII), in one sub-group of compounds:

(i) R⁴ is selected from C₁₋₆ alkyl, C₁₋₆ alkoxy and halogen; and/or
(ii) R⁸ is selected from C₁₋₆ alkyl, C₁₋₆ alkoxy and halogen.

Within this sub-group of compounds, particular compounds are those wherein R⁴ is chlorine or methoxy and R⁵ is methoxy or C₁₋₆ alkyl (for example methyl, ethyl, n-propyl, isopropyl, n-butyl, sec-butyl, tert-butyl and 2,2-dimethylpropyl).

In each of formulae (II) and (III) to (VIII), R⁴ is preferably selected from chlorine and methoxy and more preferably is chlorine.

In each of formulae (II) and (III) to (VIII), R² is preferably selected from hydrogen; amino; halogen; C₁₋₄ alkoxy; C₁₋₄ alkyl optionally substituted by fluorine, C₁₋₂ alkoxy, carbamoyl, N—C₁₋₂alkylcarbamoyl or C₁₋₂ alkoxycarbonyl; and C₁₋₄ alkylthio optionally substituted by C₁₋₂ alkoxy, carbamoyl, N—C₁₋₂ alkylcarbamoyl or C₁₋₂ alkoxycarbonyl. More preferably R² is selected from hydrogen; amino; halogen (e.g. chlorine or bromine); C₁₋₄ alkoxy (e.g. methoxy); and C₁₋₄ alkylthio (e.g. ethylthio and methylthio) and C₁₋₄ alkyl (e.g. methyl).

A particularly preferred substituent R² is chlorine.

For the avoidance of doubt, it is to be understood that each general and specific preference, embodiment and example of the group R¹ may be combined with each general and specific preference, embodiment and example of the groups R² and/or R³ and/or R⁴ and/or R⁵ and/or R⁶ and/or R⁷ and/or R⁸ and/or R¹⁰ or sub-groups thereof (e.g. R^{10a0}, R^{10a}, R^{10b} R^{10c} or R^{10cc}) as defined herein and that all such combinations are embraced by this application.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Specific compounds of the invention include:
4-(2,6-dimethoxy-phenyl)-6-methyl-pyrimidin-2-ylamine;
4-(2-chloro-6-methoxy-phenyl)-pyrimidin-2-ylamine;
4-(2-chloro-6-methoxy-phenyl)-6-methyl-pyrimidin-2-ylamine;
4-chloro-6-(2,6-dimethoxy-phenyl)-pyrimidin-2-ylamine;
6-(2,6-dimethoxy-phenyl)-pyrimidine-2,4-diamine;
4-(2,6-dichloro-phenyl)-6-methyl-pyrimidin-2-ylamine;
2-(2-amino-6-chloro-pyrimidin-4-yl)-3-methoxy-phenol;
4-chloro-6-(2-chloro-6-methoxy-phenyl)-pyrimidin-2-yl-amine;
4-(2,6-dimethoxy-phenyl)-6-ethyl-pyrimidin-2-yl-amine;
4-(2,6-dimethyl-phenyl)-6-methyl-pyrimidin-2-yl-amine;
4-bromo-6-(2-methoxy-6-propoxyphenyl)-pyrimidin-2-ylamine;
4-(2,6-difluoro-phenyl)-6-methyl-pyrimidin-2-yl-amine;
2-(2-amino-6-methyl-pyrimidin-4-yl)-3-methoxy-phenol;
5-bromo-4-(2,6-dimethoxy-phenyl)-6-methyl-pyrimidin-2-yl-amine;
4-(2-chloro-6-methoxy-phenyl)-6-methoxy-pyrimidin-2-yl-amine;
4-chloro-6-(2-fluoro-6-methoxy-phenyl)-pyrimidin-2-yl-amine;
4-chloro-6-(2,5-dimethoxy-phenyl)-pyrimidin-2-yl-amine;
4-(2-benzyloxy-6-methoxy-phenyl)-6-chloro-pyrimidin-2-yl-amine;
[2-(2-amino-6-chloro-pyrimidin-4-yl)-3-methoxy-phenoxy]-acetic acid ethyl ester;
[2-amino-6-(2,6-dimethoxy-phenyl)-pyrimidin-4-ylsulphanyl]-acetic acid ethyl ester;
4-chloro-6-[2-methoxy-6-(tetrahydro-pyran-4-ylmethoxy)-phenyl]-pyrimidin-2-yl-amine;
4-[2-methoxy-6-(2-morpholin-4-yl-ethoxy)-phenyl]-pyrimidin-2-yl-amine;
4-(2,6-dimethoxy-phenyl)-6-ethylsulphanyl-pyrimidin-2-yl-amine;
3-[2-amino-6-(2,6-dimethoxy-phenyl)-pyrimidin-4-yl]-N-ethyl-propionamide;
4-chloro-6-[2-methoxy-6-(pyridin-4-ylmethoxy)-phenyl]-pyrimidin-2-yl-amine;
4-chloro-6-(2-chloro-6-fluoro-phenyl)-pyrimidin-2-yl-amine;
4-chloro-6-(2,4,6-trimethoxy-phenyl)-pyrimidin-2-yl-amine;
4-chloro-6-(6-chloro-2-fluoro-3-methyl-phenyl)-pyrimidin-2-yl-amine;
4-(2-methoxy-phenyl)-pyrimidin-2-yl-amine;
4-(2-methoxy-5-pyridin-3-yl-phenyl)-pyrimidin-2-yl-amine;
4-(2-methoxy-phenyl)-6-methyl-pyrimidin-2-yl-amine;
4-chloro-6-(2-methoxy-phenyl)-pyrimidin-2-yl-amine;
4-(2-chloro-phenyl)-6-methyl-[1,3,5]triazin-2-yl-amine;
4-biphenyl-2-yl-6-methyl-pyrimidin-2-ylamine;
4-methyl-6-(2-morpholin-4-yl-methyl-phenyl)-pyrimidin-2-ylamine;
4-(3-amino-phenyl)-6-methyl-pyrimidin-2-yl-amine;
4-chloro-6-(2-chloro-phenyl)-pyrimidin-2-yl-amine;
N-[3-(2-amino-6-methyl-pyrimidin-4-yl)-phenyl]-benzamide;
4-chloro-6-(2-isopropyl-phenyl)-pyrimidin-2-yl-amine;
N-[3-(2-amino-6-methyl-pyrimidin-4-yl)-phenyl]-2-methoxy-acetamide;
4-methyl-6-pyridin-3-yl-pyrimidin-2-yl-amine;
4-chloro-6-(2,4-dichloro-phenyl)-pyrimidin-2-yl-amine; and
4-chloro-6-(2-chloro-4-ethoxy-phenyl)-pyrimidin-2-yl-amine;
4-chloro-6-(2-ethoxy-naphthalen-1-yl)-pyrimidin-2-ylamine;
4-chloro-6-(2,4-dimethoxy-biphenyl-3-yl)-pyrimidin-2-ylamine;
4-(3′-bromo-2,4,2′,6′-tetramethoxy-biphenyl-3-yl)-6-chloro-pyrimidin-2-ylamine;
4-chloro-6-(2-isobutoxy-6-methoxy-phenyl)-pyrimidin-2-ylamine;
4-chloro-6-[2-methoxy-6-(2-morpholin-4-yl-ethoxy)-phenyl]-pyrimidin-2-ylamine;
4-chloro-6-(2-chloro-5-methoxy-phenyl)-pyrimidin-2-ylamine;
4-chloro-6-(3-chloro-pyridin-4-yl)-pyrimidin-2-ylamine;
4-chloro-6-(2-methoxy-naphthalen-1-yl)-pyrimidin-2-ylamine;
4-chloro-6-[2-methoxy-6-(2-piperidin-1-yl-ethoxy)-phenyl]-pyrimidin-2-ylamine;
{3-[2-(2-amino-6-chloro-pyrimidin-4-yl)-3-chloro-phenoxy]-propyl}-carbamic acid tert-butyl ester;
4-chloro-6-[2-chloro-6-(3-dimethylamino-propoxy)-phenyl]-pyrimidin-2-yl-amine;
4-chloro-6-[2-chloro-6-(2-dimethylamino-ethoxy)-phenyl]-pyrimidin-2-yl amine;
4-[2-(2-amino-ethoxy)-6-chloro-phenyl]-6-chloro-pyrimidin-2-yl-amine;
{2-[2-(2-amino-6-chloro-pyrimidin-4-yl)-3-chloro-phenoxy]-ethyl}-carbamic acid tert-butyl ester;
4-difluoro-methyl-6-(2-methoxy-phenyl)-pyrimidin-2-ylamine;
4-chloro-6-(2,4-dichloro-6-methoxy-phenyl)-pyrimidin-2-ylamine;
4-(2,4-dichloro-phenyl)-pyrimidin-2-ylamine;
4-chloro-6-(2,4-dichloro-5-methoxy-phenyl)-pyrimidin-2-ylamine;
5-bromo-4-(2,4-dichloro-phenyl)-pyrimidin-2-ylamine;
4-(2,4-dichloro-phenyl)-5-(1H-pyrazol-4-yl)-pyrimidin-2-ylamine;
4-(2-amino-6-chloro-pyrimidin-4-yl)-3-chloro-phenol;
4-chloro-6-(2-chloro-4-isopropoxy-phenyl)-pyrimidin-2-ylamine;
[4-(2-amino-6-chloropyrimidin-4-yl)-3-chloro-phenoxy]-acetic acid ethyl ester;
2-[4-(2-amino-6-chloro-pyrimidin-4-yl)-3-chloro-phenoxy]-propionic acid ethyl ester;
2-[4-(2-amino-6-chloro-pyrimidin-4-yl)-3-chloro-phenoxy]-2-methyl-propionic acid ethyl ester;
4-chloro-6-(2-chloro-3-methoxy-phenyl)-pyrimidin-2-yl-amine;
4-chloro-6-(2,3-dimethoxy-phenyl)-pyrimidin-2-yl-amine;
4-chloro-6-[3-(2,2-dimethyl-propyl)-2-methoxy-phenyl]-pyrimidin-2-yl-amine;
4-chloro-6-[2-chloro-3-(2,2-dimethyl-propyl)phenyl]-pyrimidin-2-yl-amine;
4-chloro-6-(2-chloro-4-methoxy-phenyl)-pyrimidin-2-ylamine;
4-chloro-6-[2-chloro-4-(2-dimethylamino-ethoxy)-phenyl]-pyrimidin-2-yl amine;
2-[4-(2-amino-6-chloro-pyrimidin-4-yl)-3-chloro-phenoxy]-propionitrile;
4-chloro-6-(2-chloro-3-methyl-phenyl)-pyrimidin-2-yl-amine;
5-(2-amino-6-chloro-pyrimidin-4-yl)-2,4-dichloro-phenol;
4-(5-benzyloxy-2,4-dichloro-phenyl)-6-chloro-pyrimidin-2-yl-amine;
4-chloro-6-[2,4-dichloro-5-(2-diethylamino-ethoxy)-phenyl]-pyrimidin-2-yl-amine;
4-chloro-6-[2,4-dichloro-5-(2-morpholin-4-yl-ethoxy)-phenyl]-pyrimidin-2-yl-amine;
2-[5-(2-amino-6-chloro-pyrimidin-4-yl)-2,4-dichloro-phenoxy]-acetamide;
4-{2-[4-(2-amino-6-chloro-pyrimidin-4-yl)-3-chloro-phenoxy]-2-methyl-propionyloxy-methyl}-piperidine-1-carboxylic acid tert-butyl ester;
2-[4-(2-amino-6-chloro-pyrimidin-4-yl)-3-chloro-phenoxy]-2-methyl-propionic acid piperidin-4-ylmethyl ester
   and salts, solvates, N-oxides and tautomers thereof.

### Salts Solvates, Tautomers, Isomers, N-Oxides, Esters, Prodrugs and Isotopes

A reference to a compound of the formula (I) and sub-groups thereof also includes ionic forms, salts, solvates, isomers, tautomers, N-oxides, esters, prodrugs, isotopes and protected forms thereof, for example, as discussed below; preferably, the salts or tautomers or isomers or N-oxides or solvates thereof; and more preferably, the salts or tautomers or N-oxides or solvates thereof.

Many compounds of the formula (I) can exist in the form of salts, for example acid addition salts or, in certain cases salts of organic and inorganic bases such as phenolate, carboxylate, sulphonate and phosphate salts. All such salts are within the scope of this invention, and references to compounds of the formula (I) include the salt forms of the compounds.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, nonaqueous media such as ether, ethyl acetate, ethanol, isopropanol, or acetonitrile are used.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, (+)-L-lactic, (±)-DL-lactic, lactobionic, maleic, malic, (−)-L-malic, malonic, (±)-DL-mandelic, methanesulphonic, naphthalene-2-sulphonic, naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, p-toluenesulphonic, undecylenic and valeric acids, as well as acylated amino acids and cation exchange resins.

If the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth metal cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

Examples of tautomeric forms include, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

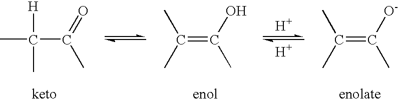

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures (e.g. racemic mixtures) or two or more optical isomers, unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

As an alternative to chiral chromatography, optical isomers can be separated by forming diastereoisomeric salts with chiral acids such as (+)-tartaric acid, (−)-pyroglutamic acid, (−)-di-toluoyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastercoisomer). In one general embodiment, 99% or more (e.g. substantially all) of the total amount of the compound of the formula (I) may be present as a single optical isomer (e.g. enantiomer or diastereoisomer).

The compounds of the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters and acyloxy esters of the compounds of formula (I) bearing a carboxylic acid group or a hydroxyl group are also embraced by Formula (I). Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(—O)OCH₃, —C(—O)OCH₂CH₃, —C(—O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl
(e.g., -Me, -Et, -nPr, -iPr, -nBu, -sBu, -iBu, -tBu);
C₁₋₇ aminoalkyl
(e.g., aminoethyl; 2-(N,N-diethylamino)ethyl; 2-(4-morpholino)ethyl); and
acyloxy-C₁₋₇alkyl
(e.g., acyloxymethyl;
acyloxyethyl;
pivaloyloxymethyl;
acetoxymethyl;
1-acetoxyethyl;
1-(1-methoxy-1-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl;
(4-tetrahydropyranyloxy) carbonyloxymethyl;
1-(4-tetrahydropyranyloxy)carbonyloxyethyl;
(4-tetrahydropyranyl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Biological Activity

The compounds of the formulae (I) and sub-groups thereof are inhibitors of Hsp90 and consequently are expected to be beneficial in the treatment of wide spectrum of proliferative disorders. Examples of such proliferative disorders include, but are not limited to, a carcinoma, for example a carcinoma of the bladder, breast, colon (e.g. colorectal carcinomas such as colon adenocarcinoma and colon adenoma), kidney, epidermis, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, gastrointestinal system, e.g. gastrointestinal stromal tumours, or skin, for example squamous cell carcinoma; a hematopoieitic tumour of lymphoid lineage, for example leukaemia, acute lymphocytic leukaemia, chronic lymphocytic leukaemia, B-cell lymphoma (such as diffuse large B cell lymphoma), T-cell lymphoma, Hodgkin's lymphoma, non-Hodgkin's lymphoma, hairy cell lymphoma, or Burkett's lymphoma; a hematopoieitic tumour of myeloid lineage, for example acute chronic myelogenous leukaemias, Imatinib sensitive and refractory chronic myelogenous leukaemias, myelodysplastic syndrome, Bortezomib sensitive and refractory multiple myeloma, myeloproliferative disease or promyelocytic leukaemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xeroderma pigmentosum; keratoacanthoma; thyroid follicular cancer; or Kaposi's sarcoma.

The cancers may be cancers which are sensitive to Hsp90 inhibition, and such cancers may be determined by a method as set out in the section headed “Methods of Diagnosis”.

One group of cancers includes human breast cancers (e.g. primary breast tumours, node-negative breast cancer, invasive duct adenocarcinomas of the breast, non-endometrioid breast cancers); and mantle cell lymphomas. In addition, other cancers are colorectal and endometrial cancers.

Another sub-set of cancers includes hematopoietic tumours of lymphoid lineage, for example leukemia, chronic lymphocytic leukaemia, mantle cell lymphoma and B-cell lymphoma (such as diffuse large B cell lymphoma) and optionally further includes chronic myelogenous leukaemia and multiple myeloma.

A preferred sub-set of cancers consists of ErbB2-positive breast, prostate, lung, and gastric cancer; chronic myeloid leukemia; androgen receptor dependent prostate cancer; Flt3-dependent acute myeloid leukaemia; melanoma associated with Braf mutation; multiple myeloma; velcade refractory multiple myeloma; and gastrointestinal stromal tumours (GIST).

Of these, particularly preferred cancers are multiple myleomas and velcade refractory tumour types as defined herein.

Hsp90 inhibitors could also be used to treat other conditions such as viral infections, parasitic disease, autoimmune diseases, neuro-degenerative disorders, inflammation, Type I and II diabetes, and cardiac disease.

Hsp90 inhibitors could also have clinical benefit in transplantation and immunosuppression.

Hsp90 inhibitors may also have clinical benefit in the previously described diseases when used in combination with existing or new therapeutic agents.

Based on the activities of Hsp90 client proteins and experimental evidence, the following disorders may be particularly sensitive to treatment by Hsp90 inhibitors.

### ErbB2-Positive Breast, Prostate, Lung, and Gastric Cancer

Overexpression of ErbB2 (HER-2) occurs in approximately 30% of breast cancers and ErbB2 receptor down-regulation by herceptin sensitized cells to Taxol. ErbB2 over-expression is linked to poor prognosis and drug resistance (Tsugawa et. al., 1993. Oncology 1993; 50: 418).

### Mutant EGFR in Lung Cancer

Somatic mutations in the kinase domain of the epidermal growth factor receptor (EGFR), including L858R and exon 19 deletions, underlie responsiveness to gefitinib and erlotinib in non-small cell lung cancer (NSCLC). Acquired resistance to these tyrosine kinase inhibitors is in some cases mediated by a second mutation, T790M. Ansamycin antibiotics, such as geldanamycin, potently inhibit heat shock protein 90 (Hsp90), promoting ubiquitin-mediated degradation of oncogenic kinases that require the chaperone for proper conformational folding. Exposure of EGFR-mutant cell lines to geldanamycin induced marked depletion of phospho-Akt and cyclin D1 as well as apoptosis. These data suggest mutational activation of EGFR is associated with dependence on Hsp90 for stability and that Hsp90 inhibition may represent a novel strategy for the treatment of EGFR-mutant NSCLC.

### Chronic Myeloid Leukemia

The aberrant BCR-Abl protein is created through a chromosomal translocation and results in a constitutively active Abl kinase domain. This translocation event has been shown to be causal for CML. P210BcrAbl is a known client protein for Hsp90. Treatment of the BCR-Abl cell line K562 with an hsp90 inhibitor induced apoptosis. The Bcr-Abl inhibitor Gleevec® also induces apoptosis in K562 cells; however Gleevec® resistant K562 cells still retain sensitivity towards Hsp90 inhibitors (Gorre et. al. 2002, Blood 100: 3041-3044).

### Androgen Receptor Dependent Prostate Cancer

The androgen receptor kinase is an Hsp90 client protein. Hormone replacement therapy is usually adopted where surgery does not resolve the cancer. Eventually through mutation in the receptor the cancer becomes refractory to the hormone analogue. Hsp90 regulation of the receptor would still be viable post-mutation.

The same would apply to estrogen-dependent breast cancers.

### Flt3-Dependent Acute Myeloid Leukaemia

Internal duplication of the tyrosine kinase receptor Flt3 leads to its constitutive activation and oncogenesis. These internal duplications are observed in 20% of all reported cases of AML and are an indication of poor prognosis. Much like the activation of the ABL kinase in CML, this represents another example of a single genetic lesion giving rise to a malignancy. Hsp90 inhibitors are predicted to be of clinical benefit to these patients as Flt3 is an Hsp90 client protein (Bali et. al., 2004 Cancer Res. 64(10):3645-52).

Melanoma Associated with Braf Mutation

Braf encodes for a serine/threonine kinase which is mutated in 70% of all melanomas. 80% of these represent a single V599E point mutation that confers elevated kinase activity to BRAF. This mutation is also transforming in NIH3T3 cells (Bignell et. al., 2002 Nature. 417(6892):949-54).

### Multiple Myeloma

The Hsp90 inhibitor 17-AAG potently inhibits proliferation of Bortezomib refractory multiple myeloma cell lines. Cell surface levels of IGF-LR and IL-6R were also diminished in 17-aag treated MM-1 cells (Mitsiades et. al., Blood 107:1092-1100, 2006). Autocrine stimulation of multiple myeloma cells, as well as paracrine stimulation of bone marrow stromal cells with IL-6 is also diminished through downregulation of the Hsp90 client IKK.

### Velcade Refractory Multiple Myeloma

Compounds of the present invention can be used in the treatment of velcade refractory tumour types including treatment of patients with second line mantle cell lymphoma, indolent non-Hodgkin's lymphoma, stage IIIB and IV Bronchioloalveolar carcinoma, advanced non-small cell lung cancer, breast, prostate and ovarian cancers and non-Hodgkin's lymphoma.

### Gastrointestinal Stromal Tumours (GIST)

GIST disease particularly disease dependent on growth factor activation or overexpression (e.g. c-kit)

Other conditions or disorders for which an Hsp90 inhibitor may be of clinical benefit include, but are not limited to:

### Neurodegenerative Disorders

Huntington's disease (HD) is a progressive neurodegenerative disorder with no effective treatment. GA inhibition of Hsp90 and the resulting up-regulation of Hsps are effective in preventing huntington protein aggregation in neuronal cells. (Sittler et. al., 2001, Human Molecular Genetics, Vol. 10, No. 12 1307-1315). Up-regulation of HSP may also be of clinical benefit in other diseases of protein misfolding e.g., CJD and Alzheimer's.

### Inflammatory Disease, Including Rheumatoid Arthritis, Asthma, Chronic Obstructive Pulmonary Disease, and Inflammatory Bowel Disease

GA has been shown to dissociate HSF-1 from Hsp90 leading to the activation and nuclear translocation of HSF-1. HSF-1 subsequently acts as a transcription factor to induce HSP90 and Hsp70. The induction of Hsp70 has been implicated in the resolution of inflammation in an induced mouse model of edema (Ianaro et al., 2004 Human Molecular Genetics, 2001, Vol. 10, No. 12 1307-1315). Additionally GA treatment inhibited IkappaB kinase (IKK) activation by TNF-a or PMA. IkBa is a regulator of Nf-kB and Ap-1. (Broemer et. al. 2004). Ap-1 and Nf-kB is a major transcription factor leading to the production of pro-inflammatory cytokines (Yeo et. al., 2004 Biochem Biophys Res Commun. 30; 320(3):816-24). The stability of pro-inflammatory cytokine transcripts is also regulated through inhibition of p38 MapK (Wax et. al., 2003. Rheumatism Vol. 48, No. 2, pp 541-550).

### Angiogenesis Related Disease, Including But not Limited to: Tumour Angiogenesis, Psoriasis, Rheumatoid Arthritis, and Diabetic Retinopathy

Induction of angiogenesis is regulated by Hsp90 client proteins eNOS and Akt in endothelial cells (Sun and Liao, 2004 Arterioscler Thromb Vasc Biol. 24(12):2238-44). Suppression of hypoxia-inducible factor (HIF)-1a can also impair the growth, angiogenesis and vessel maturation of gastric tumours in a mouse model. (Stoeltzing et. al., 2004 J Natl Cancer Inst; 96:946-956.).

### Type I and Type II Diabetes

Hsp90 inhibition has a profound effect on Akt signalling as well as e-nos. These are two key regulators in high glucose induced endothelial cell apoptosis in type I diabetes (Lin et. al., 2005 J Cell Biochem. 1; 94(1):194-201) and the development of hypertension in type II diabetes (Kobayashi et. al., 2004 Hypertension. 44(6):956-62.).

### Immunosuppression and Transplantation

Hsp90 inhibition has been shown to down regulate Lck, a T-cell specific tyrosine kinase required for T-cell activation. (Yorgin et. al., 2000 J Immunol. 15; 164(6):2915-23.)

### Cardiac Disease

Cardiac ischemic is the most common cause of death in the western world. Hsps, and notably Hsp70 (induced by radicicol treatment) have demonstrated cardioprotective activity in rat cardiomyocytes (Griffin et. al., 2004). Inhibition of Hsp90 results in the release of HSF-1 from the chaperone complex and its subsequent activation of Hsp genes. Inhibition of Hsp90 also leads to the down-regulation of HIF-1, which has been implicated in the pathogenesis of ischemic heart disease and stroke.

### Infectious Disease

Hepatits C viral NS2/3 protease is an Hsp90 client protein and Hsp90 activity is required for viral processing and replication (Whitney et. al., 2001. Proc Natl Acad Sci USA. 20; 98(24):13931-5.).

### Parasitic Disease

GA has reported antimalarial activity against an Hsp90 ortholog of Plasmodium falciparum. Plasmodium growth was inhibited with GA at an IC₅₀ similar to that observed with chloroquine. GA was also effective against chloroquine resistant strains of Plasmodium falciparum (Kamar et. al., 2003. Malar J. 15; 2(1):30).

The biological activity of the compounds of the invention, e.g. as inhibitors of Hsp90, can be measured using the assays set forth in the examples below, for example the isothermal titration calorimetry (ITC) experiments described in Example 80 and the anti-proliferative activity assays described in Example 81. The level of activity exhibited by a given compound in the ITC assay can be defined in terms of the K_{d} value, and preferred compounds of the present invention are compounds having a K_{d} value of less than 1 micromolar, more preferably less than 0.1 micromolar. In the anti-proliferative activity assays, the level of activity exhibited by a given compound in an assay can be defined in terms of the IC₅₀ value, and preferred compounds of the present invention are compounds having an IC₅₀ value of less than 1 micromolar, more preferably less than 0.1 micromolar.

It has also been found that many compounds of the formula (I) have low hERG activity and a good separation between Hsp90 inhibitory activity and hERG activity.

Preferred compounds of the formula (I) have mean IC₅₀ values against hERG that are greater than 30 times, or greater than 40 times, or greater than 50 times the IC₅₀ values of the compounds in cellular proliferation assays. Preferred compounds of the formula (I) have mean IC₅₀ values against hERG that are greater than 5 μM, more particularly greater than 10 μM, and more preferably greater than 15 μM. Some compounds of the invention have mean IC₅₀ values against hERG that are greater than 50 μM.

Compounds of the invention have advantageous ADME properties and in particular better tumour distribution.

### Methods for the Preparation of Compounds of the Formula (I)

In this section, as in all other sections of this application unless the context indicates otherwise, references to Formula (I) also include all sub-groups and examples thereof as defined herein. Where a reference is made to a group R¹, R², R³, R⁴, R⁵, R⁶, R¹⁰, R¹¹ or any other “R” group, the definition of the group in question is as set out above and as set out in the following sections of this application unless the context requires otherwise.

Compounds of the formula (I) can be prepared in accordance with synthetic methods well known to the skilled person.

One method of preparing compounds of the formula (I) comprises the reaction of a compound of the formula (X):

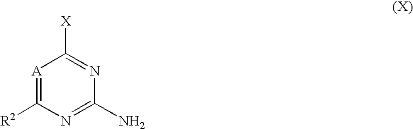

or an N-protected derivative thereof, with a compound of the formula R¹—Y; wherein one of X and Y is selected from chlorine, bromine, iodine and trifluoro-methanesulphonate; and the other of X and Y is a boronate residue such as a boronic acid group or a boronate ester, under Suzuki coupling conditions, and thereafter optionally removing any protecting groups present and optionally converting one compound of the formula (I) into another compound of the formula (I).

The foregoing process is particularly suitable when R² is hydrogen, halogen, or a nitrogen, carbon, oxygen, or sulphur linked moiety.

Compounds of the formula (X) are commercially available or can be prepared by standard methods, see for example Heterocyclic Chemistry, 3^{rd} edition, Chapman and Hall: Authors J. A. Joule, K. Mills & G. F. Smith. Compounds of the formula (X) can also be prepared from compounds of the formulae (XI) and (XV) as described below.

The Suzuki coupling can be carried out under typical Suzuki coupling conditions in the presence of a palladium catalyst such as bis(tri-t-butylphosphine)palladium and a base (e.g. a carbonate such as potassium carbonate, or potassium phosphate). Many different Suzuki coupling conditions are described in the literature, and one set of conditions found to be particularly useful in the context of the present invention is the set of conditions described in G. Fu et al. J. Am. Chem. Soc, 2002, 124, 6343. The coupling reaction may be carried out in an aqueous solvent system, for example a water; ethanol; methanol; toluene mix and the reaction mixture is typically subjected to heating, for example to a temperature of 50° C., or higher. It will be appreciated by the skilled person that when R² is halogen and the amino group on the pyrimidine or triazine ring is unprotected, careful attention will need to be paid to the relative concentrations of the reactants, the length of time over which the reaction is allowed to run, and the temperature at which the reaction is maintained, in order to prevent or minimise subsequent S_{N}Ar reactions and/or bis-couplings.

Many boronates suitable for use in preparing compounds of the invention are commercially available, for example from Aldrich, Lancaster and Acros. Where the boronates are not commercially available, they can be prepared by methods known in the art, for example as described in the review article by N. Miyaura and A. Suzuki, Chem. Rev. 1995, 95, 2457. Boronates can be prepared, for example, by reacting the appropriate H—, iodo- or bromo-compound with an alkyl lithium, such as butyl lithium, and then reacting with a borate ester. The resulting boronate ester derivative can, if desired, be hydrolysed to give the corresponding boronic acid.

In another process for preparing compounds of the formula (I), a compound of the formula (XI):

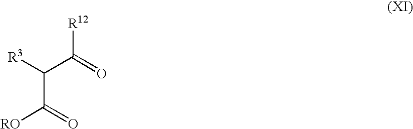

where OR is an alkoxy group such as methoxy, R¹² is a group R¹ or a group R²; and R¹, R² and R³ are as defined herein (provided that R² is other than a moiety such as halogen or amino that may interfere with the cyclisation reaction), is reacted with guanidine or a salt thereof to give a compound of the formula (XII):

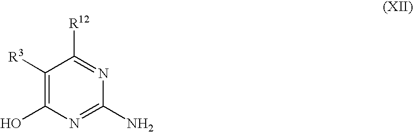

and thereafter replacing the hydroxy group in formula (XII) with a chlorine atom by reaction with a chlorinating agent to give a compound of the formula (XIII):

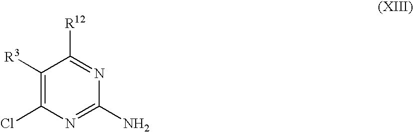

Compounds of the formula (XIII) wherein R¹² is a group R² can be reacted with R¹—Y (wherein Y is a boronate residue) under the Suzuki coupling conditions described above to give compounds of the formula (I).

When R¹² is R¹, instead of replacing the hydroxy group in formula (XII) with a chlorine atom by reaction with a chlorinating agent, the hydroxy group may be converted into a range of other functional groups corresponding to R² by standard methods well known per se to the skilled person.

Compounds of the formulae (XI) and (XII) are commercially available or can be prepared by methods well known to the skilled person, see for example Advanced Organic Chemistry, by Jerry March, the reference for which is given below.

The reaction of the β-ketoester of formula (XI) with guanidine (preferably the hydrochloride salt thereof) is typically carried out in the presence of a base such as sodium methoxide in a polar solvent such as ethanol, usually with heating, for example to the reflux temperature of the solvent.

The chlorination of the compound of formula (XII) may be carried out using phosphorus oxychloride (usually an excess) or phosphorus pentachloride, preferably with heating to a non-extreme temperature.

The chloropyrimidine of formula (XIII), wherein the pyrimidine amino group may be protected as necessary, can be used for the preparation of a range of other compounds of the formula (I) by reaction with a nucleophile suitable for introducing a group R². For example, where the nature of R² permits, the chloro compound of formula (XIII) can be reacted with a nucleophile selected from Grignard reagents, cuprates, organo tin and organo zinc reagents, amines, thiols and alkoxides. Such reactions are well known known in the literature: for Grignard coupling reactions see Furstner et al, J. Amer. Chem. Soc. 2002, 124, 13863); for related Stille couplings see Gundersen et al. Bioorg Med Chem. Letts. 2003, 13, 877); for amine and thiol nucleophilic displacement reactions see Gibson et al. Tetarahedron 2004, 60, 943. Additionally, the transformation may be carried out with an alkyl boronic acid, as described above, or, via lithiation with such reagents as butyl lithium to give a lithiated intermediate followed by alkylation (see for example Jackson et al. Aust J Chem 2002, 5513, 205). It is to be understood that other nucleophilic species could be used to generate other pyrimidine analogues of formula (I) not specifically exemplified herein.

A further process for preparing compounds of the formula (I) involves the condensation reaction of a ketone or amide compound of the formula (XIV):

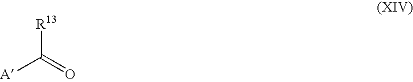

where R¹³ is a group R¹, A′ is NH₂ or CH₂R³ where R³ is hydrogen or an optionally substituted alkyl, aryl or heteroaryl group, with a compound R²C(OMe)₂NMe₂ such as N,N-dimethylacetamide dimethyl acetal to give an intermediate enamine compound of formula (XV):

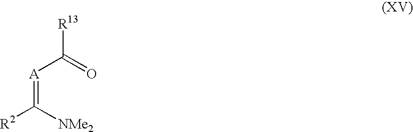

which may then be reacted with guanidine hydrochloride to give a compound of the formula (I). In this reaction sequence, R² cannot be a halogen or amino group and typically is hydrogen or an acyclic hydrocarbyl group such as an alkyl group or a substituted alkyl group, e.g a methyl group.

In a variation on the above reaction sequence, R¹³ can be a group R² (other than a halogen or amino group or other group that would interfere with the cyclisation reaction) and the group R¹ is introduced into the intermediate (XV) in place of R² by reaction of (XIV) with a compound of the formula R¹C(OMe)₂NMe₂.

The condensation reaction between the ketone (XIV) and the N,N-dimethylamide dimethyl acetal is generally performed by heating the reactants neat (where one or both of the reactants are liquids) or in a polar solvent e.g. tetrahydrofuran (THF) or methanol (MeOH) to a temperature in excess of 100° C. The resulting enamine (XV) can then be condensed with guanidine hydrochloride, by reaction in the presence of an inorganic base (e.g. sodium or potassium hydroxide or carbonate) or an organic base such as triethylamine or pyridine.

The enamine condensation route may be used to prepare both pyrimidines and triazines.

Once formed, one compound of the formula (I), or a protected derivative thereof, can be converted into another compound of the formula (I) by methods well known to the skilled person. Examples of synthetic procedures for converting one functional group into another functional group are set out in standard texts such as Advanced Organic Chemistry, by Jerry March, 4^{th} edition, 119, Wiley Interscience, New York; Fiesers' Reagentsfor Organic Synthesis, Volumes 1-17, John Wiley, edited by Mary Fieser (ISBN: 0-471-58283-2); and Organic Syntheses, Volumes 1-8, John Wiley, edited by Jeremiah P. Freeman (ISBN: 0-471-31192-8)).

By way of example, conversion of a compound wherein R¹ is a phenyl group substituted with methoxy into the corresponding phenolic compound can be carried out using boron tribromide or boron trichloride, for example in an inert solvent such as dichloromethane at room temperature under a nitrogen atmosphere (see e.g. Synthesis 1991, 469).

Phenolic compounds (R¹ is phenyl substituted by a hydroxy group) can be converted to ethers by alkylation using various standard methods of ether synthesis to give a wide range of ethers, for example compounds in which the alkylating group is a straight chain or branched alkyl group, or a saturated cyclic group such as a cycloalkyl group, or an aryl group. Phenolic compounds can also be converted to acyl derivatives (for example acyl derivatives of the type described above) by standard and well known acylation methods. In addition, phenolic compounds may also be converted to carbamates using methods well known to the skilled person.

Compound in which R¹ has an amino group substitutent can be converted to amides using standard methods of amide synthesis. Thus for example, compounds wherein R¹ is an optionally further substituted aminophenyl group can be reacted with acid chlorides or with various carboxylic acids using standard amide coupling reagents of the type commonly used in the formation of peptide linkages. Examples of such reagents include 1,3-dicyclohexylcarbodiimide (DCC) (Sheehan et al, J. Amer. Chem Soc. 1955, 77, 1067), 1-ethyl-3-(3′-dimethylaminopropyl)-carbodiimide (referred to herein either as EDC or EDAC but also known in the art as EDCI and WSCDI) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Preferred coupling reagents include EDC (EDAC) and DCC in combination with HOAt or HOBt.

Such amide coupling reactions are typically carried out in a non-aqueous, non-protic solvent such as acetonitrile, dioxane, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2(-phenylsulphonyl)ethyloxy amide (—NH-Psec). Other protecting groups for amines, such as cyclic amines and heterocyclic N—H groups, include toluenesulphonyl (tosyl) and methanesulphonyl (mesyl) groups and benzyl groups such as apara-methoxybenzyl (PMB) group. A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

### Methods of Purification

The compounds may be isolated and purified by a number of methods well known to those skilled in the art and examples of such methods include chromatographic techniques such as column chromatography (e.g. flash chromatography) and HPLC. Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

Alternatively, normal phase preparative LC based methods might be used in place of reverse phase methods. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

### Pharmaceutical Formulations

While it is possible for the active compound to be administered alone, it is preferable to present it as a pharmaceutical composition (e.g. formulation) comprising at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF).

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Accordingly, in a further aspect, the invention provides compounds of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats, co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

A drug molecule that is ionizable can be solubilized to the desired concentration by pH adjustment if the drug's pKₐ is sufficiently away from the formulation pH value. The acceptable range is pH 2-12 for intravenous and intramuscular administration, but subcutaneously the range is pH 2.7-9.0. The solution pH is controlled by either the salt form of the drug, strong acids/bases such as hydrochloric acid or sodium hydroxide, or by solutions of buffers which include but are not limited to buffering solutions formed from glycine, citrate, acetate, maleate, succinate, histidine, phosphate, tris(hydroxymethyl)-aminomethane (TRIS), or carbonate.

The combination of an aqueous solution and a water-soluble organic solvent/surfactant (i.e., a cosolvent) is often used in injectable formulations. The water-soluble organic solvents and surfactants used in injectable formulations include but are not limited to propylene glycol, ethanol, polyethylene glycol 300, polyethylene glycol 400, glycerin, dimethylacetamide (DMA), N-methyl-2-pyrrolidone (NMP; Pharmasolve), dimethylsulphoxide (DMSO), Solutol HS 15, Cremophor EL, Cremophor RH 60, and polysorbate 80. Such formulations can usually be, but are not always, diluted prior to injection.

Propylene glycol, PEG 300, ethanol, Cremophor EL, Cremophor RH 60, and polysorbate 80 are the entirely organic water-miscible solvents and surfactants used in commercially available injectable formulations and can be used in combinations with each other. The resulting organic formulations are usually diluted at least 2-fold prior to IV bolus or IV infusion.

Alternatively increased water solubility can be achieved through molecular complexation with cyclodextrins.

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer. A typical liposome formulation contains water with phospholipid at 5-20 mg/ml, an isotonicifier, a pH 5-8 buffer, and optionally cholesterol.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

The pharmaceutical formulation can be prepared by lyophilising a compound of Formula (I) or acid addition salt thereof. Lyophilisation refers to the procedure of freeze-drying a composition. Freeze-drying and lyophilisation are therefore used herein as synonyms. A typical process is to solubilise the compound and the resulting formulation is clarified, sterile filtered and aseptically transferred to containers appropriate for lyophilisation (e.g. vials). In the case of vials, they are partially stoppered with lyo-stoppers. The formulation can be cooled to freezing and subjected to lyophilisation under standard conditions and then hermetically capped forming a stable, dry lyophile formulation. The composition will typically have a low residual water content, e.g. less than 5% e.g. less than 1% by weight based on weight of the lyophile.

The lyophilisation formulation may contain other excipients for example, thickening agents, dispersing agents, buffers, antioxidants, preservatives, and tonicity adjusters. Typical buffers include phosphate, acetate, citrate and glycine. Examples of antioxidants include ascorbic acid, sodium bisulphite, sodium metabisulphite, monothioglycerol, thiourea, butylated hydroxytoluene, butylated hydroxyl anisole, and ethylenediaminetetraacetic acid salts. Preservatives may include benzoic acid and its salts, sorbic acid and its salts, alkyl esters of para-hydroxybenzoic acid, phenol, chlorobutanol, benzyl alcohol, thimerosal, benzalkonium chloride and cetylpyridinium chloride. The buffers mentioned previously, as well as dextrose and sodium chloride, can be used for tonicity adjustment if necessary.

Bulking agents are generally used in lyophilisation technology for facilitating the process and/or providing bulk and/or mechanical integrity to the lyophilized cake. Bulking agent means a freely water soluble, solid particulate diluent that when co-lyophilised with the compound or salt thereof, provides a physically stable lyophilized cake, a more optimal freeze-drying process and rapid and complete reconstitution. The bulking agent may also be utilised to make the solution isotonic.

The water-soluble bulking agent can be any of the pharmaceutically acceptable inert solid materials typically used for lyophilisation. Such bulking agents include, for example, sugars such as glucose, maltose, sucrose, and lactose; polyalcohols such as sorbitol or mannitol; amino acids such as glycine; polymers such as polyvinylpyrrolidine; and polysaccharides such as dextran.

The ratio of the weight of the bulking agent to the weight of active compound is typically within the range from about 1 to about 5, for example of about 1 to about 3, e.g. in the range of about 1 to 2.

Alternatively they can be provided in a solution form which may be concentrated and sealed in a suitable vial. Sterilisation of dosage forms may be via filtration or by autoclaving of the vials and their contents at appropriate stages of the formulation process. The supplied formulation may require further dilution or preparation before delivery for example dilution into suitable sterile infusion packs.

Extemporaneous injection solutions and suspensions may be prepared from sterile powders, granules and tablets.

In one preferred embodiment of the invention, the pharmaceutical composition is in a form suitable for i.v. administration, for example by injection or infusion.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as coin starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the formula (I) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The active compound will be administered to a patient in need thereof (for example a human or animal patient) in an amount sufficient to achieve the desired therapeutic effect.

### Methods of Treatment

It is envisaged that the compounds of the formula (I) and sub-groups as defined herein will be useful in the prophylaxis or treatment of a range of disease states or conditions mediated by Hsp90 client proteins. Examples of such disease states and conditions are set out above.

The compounds are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile or continuous manner.

A typical daily dose of the compound of formula (I) can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound can be administered on a daily basis or on a repeat basis every 2, or 3, or 4, or 5, or 6, or 7, or 10 or 14, or 21, or 28 days for example.

In one particular dosing schedule, a patient will be given an infusion of a compound for periods of one hour daily for up to ten days in particular up to five days for one week, and the treatment repeated at a desired interval such as two to four weeks, in particular every three weeks.

More particularly, a patient may be given an infusion of a compound for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

In a further particular dosing schedule, a patient is given a continuous infusion for a period of 12 hours to 5 days, an in particular a continuous infusion of 24 hours to 72 hours.

Ultimately, however, the quantity of compound administered and the type of composition used will be commensurate with the nature of the disease or physiological condition being treated and will be at the discretion of the physician.

The compounds as defined herein can be administered as the sole therapeutic agent or they can be administered in combination therapy with one of more other compounds for treatment of a particular disease state, for example a neoplastic disease such as a cancer as hereinbefore defined.

Examples of other therapeutic agents or treatments that may be administered together (whether concurrently or at different time intervals) with the compounds of the formula (I) include but are not limited to:
Topoisomerase I inhibitors
Antimetabolites
Tubulin targeting agents
DNA binder and topoisomerase II inhibitors
Alkylating Agents
Monoclonal Antibodies.
Anti-Hormones
Signal Transduction Inhibitors
Proteasome Inhibitors
DNA methyl transferases
Cytokines and retinoids
Chromatin targeted therapies, e.g. HDAC or HAT modulators
Radiotherapy.

For the case of Hsp90 inhibitors combined with other therapies, the two or more treatments may be given in individually varying dose schedules and via different routes.

Where the compound is administered in combination therapy with one, two, three, four or more other therapeutic agents (preferably one or two, more preferably one), the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through his or her common general knowledge the dosing regimes and combination therapies to use.

### Methods of Diagnosis

Prior to administration of a compound, a patient may be screened to determine whether a disease or condition from which the patient is or may be suffering is one which would be susceptible to treatment with a compound having activity against Hsp90.

For example, a biological sample taken from a patient may be analysed to determine whether a condition or disease, such as cancer, that the patient is or may be suffering from is one which is characterised by a genetic abnormality or abnormal protein expression which leads to over-activation of an Hsp90 client protein. Examples of such abnormalities that result in activation of Hsp90 client proteins include; Bcr-ABL translocation, Flt-3 internal duplication, and mutation of Braf, or over-expression of ErbB2.

Thus, the patient may be subjected to a diagnostic test to detect a marker characteristic of up-regulation. The term diagnosis includes screening. By marker we include genetic markers including, for example, the measurement of DNA composition to identify mutations of Braf, BCR-abl, and Flt3 or other affected client proteins. The term marker also includes markers which are characteristic of up regulation of ErbB2, including enzyme activity, enzyme levels. The enzyme state (e.g. phosphorylated or not) and mRNA levels of the aforementioned proteins could also be assessed to characterise a change in activity.

The diagnostic tests are typically conducted on a biological sample selected from tumour biopsy samples, blood samples (isolation and enrichment of shed tumour cells), stool biopsies, sputum, chromosome analysis, pleural fluid, peritoneal fluid, or urine.

The screening process will typically involve direct sequencing, oligonucleotide microarray analysis, or a mutant specific antibody.

Methods of identification and analysis of mutations and up-regulation of proteins are well known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3rd Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce non-specific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labelled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Commercially available FISH probes also exist for cytogenetic detection of chromosome rearrangements, which can be used to detect Flt3 and Bcr-Abl translocations within leukeamia cell populations. Standard methods for carrying out FISH are described in Ausubel, F. M. et al., eds. Current Protocols in Molecular Biology, 2004, John Wiley & Sons Inc and Fluorescence In Situ Hybridization:

Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtitre plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of the “Philadelphia chromosome” indicative of BCR-ABL translocation.

Therefore, all of these techniques could also be used to identify tumours particularly suitable for treatment with the compounds of the invention.

### EXAMPLES

The invention will now be illustrated, but not limited, by reference to the specific embodiments described in the following examples.

In the examples, the following abbreviations may be used.
AcOH acetic acid
BOC tert-butyloxycarbonyl
Bn benzyl
CDI 1,1-carbonyldiimidazole
DMAW90 Solvent mixture: DCM: MeOH, AcOH, H₂O (90:18:3:2)
DMAW120 Solvent mixture: DCM: MeOH, AcOH, H₂O (120:18:3:2)
DMAW240 Solvent mixture: DCM: MeOH, AcOH, H₂O (240:20:3:2)
DCM dichloromethane
DMF dimethylformamide
DMSO dimethyl sulphoxide
EDC 1-ethyl-3-(3′-dimethylaminopropyl)-carbodiimide
Et₃N triethylamine
EtOAc ethyl acetate
Et₂O diethyl ether
HOAt 1-hydroxyazabenzotriazole
HOBt 1-hydroxybenzotriazole
MeCN acetonitrile
MeOH methanol
P.E. petroleum ether
SiO₂ silica
TBTU N,N,N′,N′-tetramethyl-O-(benzotriazol-1-yl)uronium tetrafluoroborate
THF tetrahydrofuran

Proton magnetic resonance (¹H NMR) spectra were recorded on a Bruker AV400 instrument operating at 400.13 MHz, in DMSO-d₆ or MeOH-d₄ (as indicated) at 27° C., unless otherwise stated and are reported as follows: chemical shift δ/ppm (number of protons, multiplicity where s=singlet, d=doublet, t=triplet, q=quartet, m=multiplet, br=broad). The residual protic solvent was used as the internal reference.

In the examples, the compounds prepared were characterised by liquid chromatography and mass spectroscopy using the system and operating conditions set out below. Where atoms with different isotopes are present and a single mass quoted, the mass quoted for the compound is the monoisotopic mass (i.e. ³⁵Cl; ⁷⁹Br etc.). Different systems were used, as described below, and these were equipped with, and were set up to run under, closely similar operating conditions. The operating conditions used are also described below.

### System Description:

### System 1 (Analytical System):

| | |
|---|---|
| HPLC System: | Waters 2795 |
| Mass Spec Detector: | Micromass Platform LC |
| PDA Detector: | Waters 2996 PDA |

### System 2 (Preparative and Analytical System):

| | |
|---|---|
| HPLC System: | Waters Fractionlynx system |
| Mass Spec Detector: | Waters ZQ |
| PDA Detector: | Waters 2996 PDA |

### System 3 (Preparative and Analytical System):

| | |
|---|---|
| HPLC System: | Agilent 1100 system |
| Mass Spec Detector: | LC/MSD |
| UV Detector: | Agilent MWD |

### Operating Conditions:

### Acidic Analytical Conditions:

| | |
|---|---|
| Eluent A: | H₂O (0.1% Formic Acid) |
| Eluent B: | CH₃CN (0.1% Formic Acid) |
| Gradient: | 5-95% eluent B over 3.5 minutes (over 15 minutes w/ |
| | column 2) |
| Flow: | 0.8 ml/min |
| Column 1: | Phenomenex Synergi 4μ MAX-RP 80A, 2.0 × 50 mm |
| Column 2: | Phenomenex Synergi 4μ MAX-RP 80A, 2.0 × 150 mm |

### Basic Analytical Conditions:

| | |
|---|---|
| Eluent A: | H₂O (10 mM NH₄HCO₃ buffer adjusted to pH = 9.2 |
| | with NH₄OH) |
| Eluent B: | CH₃CN |
| Gradient: | 5-95% eluent B over 3.5 minutes |
| Flow: | 0.8 ml/min |
| Column: | Phenomenex Gemini 5μ 2.0 × 50 mm |

### MS Conditions (Waters Systems):

| | |
|---|---|
| Capillary voltage: | 3.6 kV (3.40 kV on ES negative) |
| Cone voltage: | 25 V |
| Source Temperature: | 120° C. |
| Scan Range: | 125-800 amu |
| Ionisation Mode: | ElectroSpray Positive, Negative or Positive & |
| | Negative |

### MS Conditions (Agilent Systems):

| | |
|---|---|
| Capillary voltage: | 4000 V (3500 V on ES Negative) |
| Fragmentor/Gain: | 150/1 |
| Drying gas Temp/flow: | 350° C./13.0 Lmin⁻¹ |
| Nebuliser pressure: | 50 psig |
| Scan Range: | 125-800 amu |
| Ionisation Mode: | ElectroSpray Positive or Negative |

The starting materials for each of the Examples are commercially available unless otherwise specified.

### General Synthetic Methods

### Method 1A

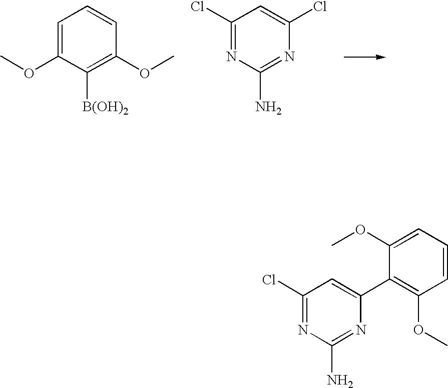

General procedure: The boronic acid (1.5 eq.), aromatic halogen (1.0 eq.), and the base K₃PO₄ (1.3 eq.) were taken up in MeOH:EtOH:toluene:water (1:1:1:1, 0.3 M) and degassed, and then Pd(tBU₃P)₂ (0.02 eq.) was added and the degassing was continued. The reaction mixture was heated thermally for 2 hours at 50° C. The reaction mixture was diluted with NaOH (1 M) and EtOAc, and the product was extracted with EtOAc (×³). The combined organic layers were washed with brine and dried over MgSO₄ and the product was filtered and evaporated to dryness. Purification by column chromatography, or preparative LCMS, gave the product.

### Method 1B

Method 1A was used with the following exceptions: boronic acid (2.4 eq.), aromatic halogen (1.0 eq.), base: K₃PO₄ (6.0 eq.), solvent (0.3 M). The reaction mixture was heated in a microwave for 30 minutes at 135° C.

### Method 1C

Method 1A was used with the following exceptions: boronic acid (2.4 eq.), aromatic halogen (1.0 eq.), base: K₃PO₄ (6.0 eq.), solvent (0.3 M). The reaction mixture was heated in the microwave for 15 minutes at 100° C.

### Method 1D

Method 1A was used with the following exceptions: boronic acid (0.5 eq.), aromatic halogen (1.0 eq.), base: K₃PO₄ (1.3 eq.), solvent (0.3 M). The reaction mixture was heated in the microwave for 15 minutes at 100° C.

### Method 1E

Method 1A was used with the following exceptions: boronic acid (1.1 eq.), aromatic halogen (1.0 eq.), base: K₂CO₃ (2.0 eq.), solvent (0.3 M). The reaction was heated in the microwave for 30 minutes at 130° C.

### Method 1F

Method 1A was used with the following exceptions: K₂CO₃ as the base, EtOH (0.15 M):water:toluene (2:1:1) for the solvent . The reaction was heated thermally overnight at 90° C.

### Method 1G

Method 1A was used with the following exceptions: K₂CO₃ as the base, EtOH (0.15 M):water:toluene (2:1:1) as the solvent. The reaction was heated in the microwave at 50° C. for 2 hours.

### Method 1H

Method 1A was used with the following exceptions: K₂CO₃ as the base, 1,4-dioxane as the solvent, and the catalyst Pd (PPh₃)₃Cl₂. The reaction was heated thermally at 50° C. for 2 hours.

### Method 2A

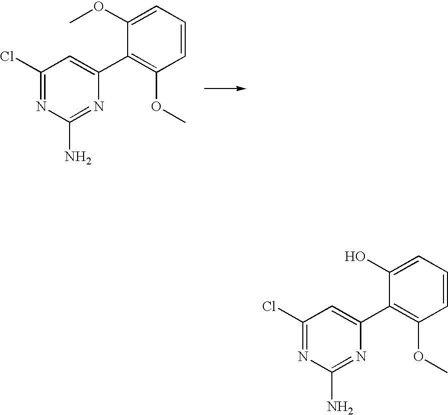

General procedure: BCl₃ (1.1 eq.) was added to the pyrimidine in DCM (0.2 M) at 0° C. under N₂. The reaction was left O/N to warm to room temperature. An equivalent of BCl₃ was added every 24 hours until the reaction was complete. The reaction was quenched with water and the product extracted with EtOAc (×3). The combined organic layers were washed with brine and dried over MgSO₄. The product was filtered and evaporated to dryness. Purification by column chromatography, or prep LCMS, gave the product.

### Method 2B

Method 2A was used with BBr₃ instead of BCl₃.

### Method 2C

To the methoxy/ethoxy compound was added minimal neat CH₂SO₄. The reaction was warmed to 40° C. for 16 hours and cooled. The reaction was diluted with ice-water and neutralised with NaOH (1M). The product was extracted with EtOAc (×3). The combined organic layers were washed with brine and dried with MgSO₄. The product was filtered and evaporated to dryness to leave a yellow solid.

### Method 3A

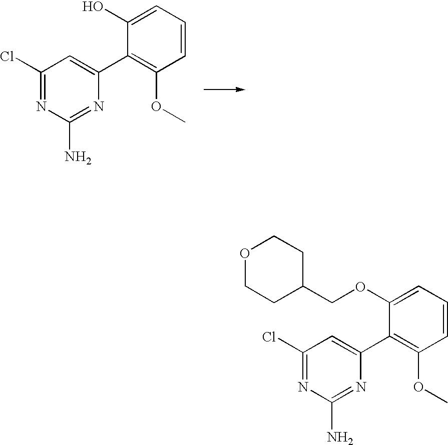

General procedure: The phenol (1.0 eq.) and the base sodium ethoxide (1.2 eq.) were taken up in a DMSO (0.3 M) under N₂. The alkylating agent was added at room temperature, and the reaction was left to stir until the reaction was complete. The reaction was worked up by pouring into water and extracting with EtOAc (×2). The combined organic layers were washed with brine and dried over MgSO₄. The product was filtered and evaporated to dryness. Purification by column chromatography, or prep LCMS, gave the product.

### Method 3B

Method 3A was used with the following exceptions: phenol (1.0 eq.), base—cesium carbonate (1.2 eq.), solvent DMF (0.3 M).

### Method 3C

Method 3A was used with the following exceptions: phenol (1.0 eq.), base—1K₂CO₃ (1.5 eq.), solvent DMF (0.3 M) at 80° C.

### Method 3D

Method 3A was used with the following exceptions: phenol (1.0 eq.), base—cesium carbonate (3.0 eq.), solvent DMF (0.3 M).

### Method 4

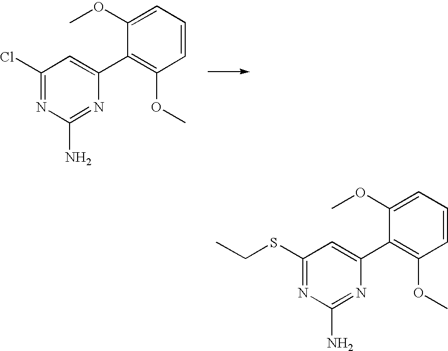

General procedure: The chloropyrimidine (1.0 eq.) and a thiol (1.5 eq.) were taken up in water:MeCN (1:1, 0.2 M) and was allowed to stir at RT overnight. The reaction mixture was warmed to 50° C. and the reaction left until complete (further equivalents of thiol were added if necessary). The product was extracted with EtOAc (×2). The combined organic layers were washed with brine and dried over MgSO₄. The product was filtered and evaporated to dryness. Purification by column chromatography gave the product.

### Method 5A

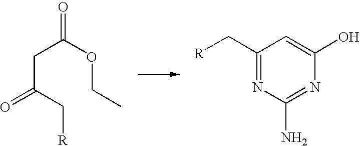

General procedure: The 13-ketoester (1.0 eq.), guanidine HCl (1.1 eq.) and sodium methoxide (2.0 eq.) in ethanol (0.3 M) were heated at reflux for 2 hours and then filtered hot and evaporated to dryness to yield the hydroxypyrimidine.

### Method 5B

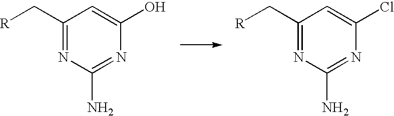

General procedure: The hydroxypyrimidine was taken up in POCl₃ (0.5 M) and heated to 70° C. for 2 hours. The reaction mixture was then allowed to cool and evaporated to dryness. The reaction was worked up by pouring into water and extracting with EtOAc (×2). The combined organic layers were washed with brine and dried over MgSO₄. The product was filtered and evaporated to dryness to yield the chloropyrimidine.

### Method 6

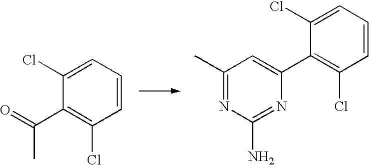

General procedure: A mixture of the ketone (1.0 eq.) and N,N-dimethylacetamide dimethylacetal (1.0 eq.) were heated together in a microwave at 180° C. for 10 minutes. The resulting gum was re-dissolved in EtOH (0.5 M), guanidine HCl (3.0 eq.) added and the mixture was heated in the microwave at 180° C. for 30 minutes. The reaction mixture was worked up by pouring into water and extracting with CH₂Cl₂ (×2) and dried over MgSO₄. The product was filtered and evaporated to dryness. Purification by column chromatography gave the product.

### Method 7

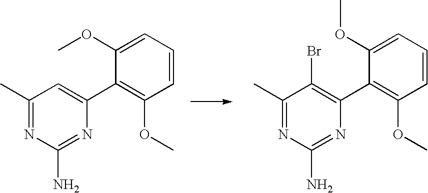

The pyrimidine (1.0 eq.), N-bromosuccinamide (1.1 eq.) and benzoyl peroxide (10 mg) in C₆H₆ (0.3 M) were heated at 100° C. for 3 hours. The reaction mixture was worked up by pouring into water and extracting with CH₂Cl₂ (×2) and dried over MgSO₄. The product was filtered and evaporated to dryness. Purification by column chromatography gave the product.

### Method 8

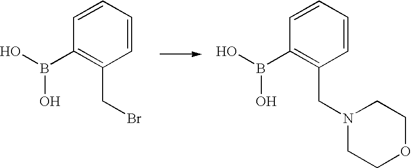

The bromide (1.0 eq.) and a morpholine (1.0 eq.) in toluene (0.5 M) were heated in a microwave at 100° C. for 5 minutes. The resulting gum was used in subsequent steps without further purification.

### Method 9

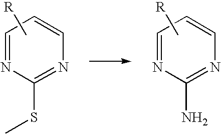

General procedure: The pyrimidine (1.0 eq.) and potassium peroxymonosulphate (Oxone®) (1.0 eq.) in MeOH:water (2:1, 0.5 M) were stirred at room temperature for 1 hour. The reaction was worked up by pouring into water and extracting with CH₂Cl₂ (×2) and dried over MgSO₄. The product was filtered and evaporated to dryness. The resulting mixture was re-dissolved in dioxane (0.5 M) and 0.88 M aqueous ammonia (0.5 M) added and then heated in the microwave at 100° C. for 30 minutes. The reaction was worked up by pouring into water and extracting with CH₂Cl₂ (×2) and dried over MgSO₄. The product was filtered and evaporated to dryness. Purification by column chromatography gave the product.

### Method 10

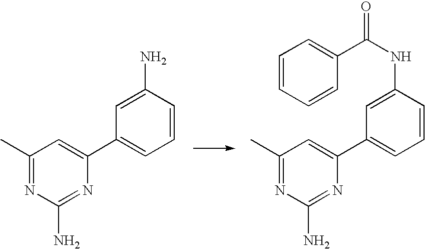

General procedure: The pyrimidine (1.0 eq.), acid chloride (1.0 eq.) and triethylamine (1.0 eq.) in DMF (0.3 M) were stirred at 50° C. for 3 hours. The reaction was worked up by pouring into water and extracting with CH₂Cl₂ (×2) and dried over MgSO₄. The product was filtered and evaporated to dryness. Purification by column chromatography gave the product.

### Method 11

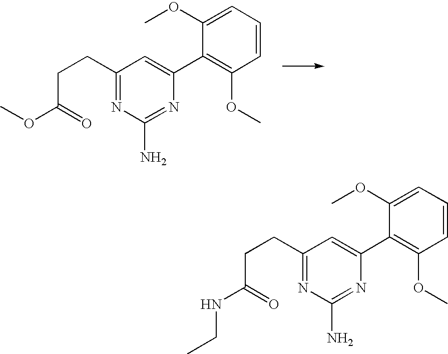

General procedure: The ester was heated with methylamine in MeOH (0.06 M) in the microwave at 135° C. for 30 minutes. The reaction was evaporated to dryness. Purification by column chromatography gave the product.

### Method 12A

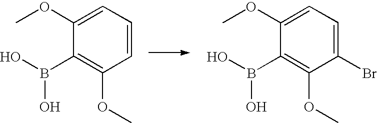

General procedure: The boronic acid in MeCN (0.3 M) was added NBS (1.1 eq.) in portions at RT. The reaction was stirred at room temperature overnight. The reaction was diluted with water and the product extracted with EtOAc (×3). The combined organic layers were washed with brine and dried over MgSO₄. The product was filtered and evaporated to dryness to leave the product. Purification by column chromatography if required.

### Method 12B

Method 12A was used except NCS was substituted for NBS.

### Method 13A

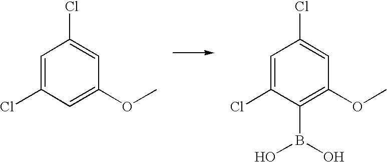

General procedure: To the benzene in THF (0.4 M) at −100° C. (liq. N₂ and 1:1 MeOH:acetone) was added the n-BuLi (1.1 eq, 1.6 M) over 20 minutes. The reaction was stirred for a further 45 minutes. B(OMe)₃ was added slowly in ether over 20 minutes. The reaction was allowed to warm to RT over 2 hours. The reaction was quenched with ice and diluted with 2M HCl and EtOAc. The reaction was stirred for 30 minutes and then the product was extracted with EtOAc (×3). The combined organic layers were dried with brine and then MgSO₄. The product was filtered and evaporated to dryness to leave a colourless oil/solid. The residue was taken on as is.

### Method 13B

Method 13A was used except the base was s-BuLi.

### Method 13C

To the benzene in THF (0.4 M) at −78° was added the n-BuLi (1.1 eq, 1.6 M) over 20 minutes. The reaction was allowed to warm to RT and was stirred for 16 hours. B(OiPr)₃ was added slowly. Work up as for Method 13A.

### Method 14

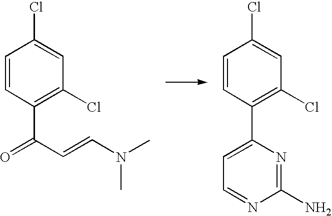

General procedure: The enone (1.0 eq.), guanidine HCl (1.1 eq.) and sodium ethoxide (1.1 eq.) in ethanol (0.2 M) were heated at reflux until the reaction was complete. The reaction was worked up by adding water and extracting with EtOAc (×2). The combined organic layers were washed with brine and dried over MgSO₄. The product was filtered and evaporated to dryness to leave a very pale yellow solid. Purification by column chromatography, or prep LCMS, gave the product.

### Method 15

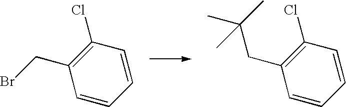

To the bromide in hexanes (0.3 M) at −78° C. was added the t-BuLi (1.1 eq.), under N₂. The reaction was allowed to warm to RT slowly. The reaction was left to stir for 16 hours and colourless slurry formed. The reaction was worked up by adding NH₄Cl (aq.), diluting with water, and extracting with EtOAc (×2). The combined organic layers were washed with brine and dried over MgSO₄. The product was filtered and evaporated to dryness to leave a pale yellow oil. Purified by column chromatography (petrol) to yield the product as a colourless oil.

### Method 16

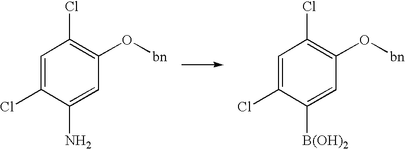

HCl (19 ml, 6M) was added to 5-(benzyloxy)-2,4-dichloroaniline (19 mmol) in acetic acid (77 ml) and the mixture was cooled in an ice-water bath. Sodium nitrite (22 mmol) was added in water (64 ml) slowly keeping the temp <5° C. This was stirred for 30 minutes. This mixture was poured in KI (39 mmol) and 12 (5.4 mmol) in water (241 ml) and was left to stir for 1.5 hours at room temperature. The reaction was worked up by adding water and extracting with DCM (×3). brown oil. The combined organic layers were washed with brine and dried over MgSO₄. The product was filtered and evaporated to dryness to leave the iodide product as a brown oil. To the iodide (12 mmol) in THF (40 ml) at −78° C. was added triisopropylborate (17 mmol) and then the n-BuLi (1.5 eq.) slowly. The reaction was allowed to stir for an hour and then quenched with NH₄Cl (sat., aq). The reaction was allowed to warm to room temperature and was diluted with water and EtOAc. The product was extracted with EtOAc (×3). The combined organic layers were washed with brine and dried over MgSO₄. The product was filtered and evaporated to dryness to leave the boronic acid as a pale yellow oil.

### Method 17

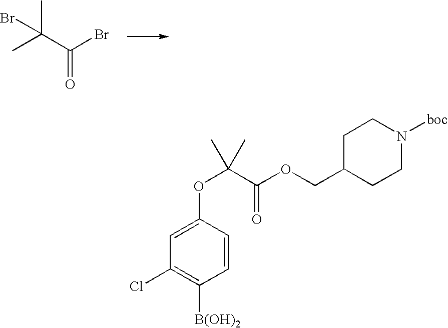

To 2-bromoisobutyryl bromide (2.1 mmol) and Et₃N (1.2 eq.) in DCM (0.1 M) at 0° C. was added BOC-isonicot(6H)-ol (1.2 eq.) slowly, under N₂. The reaction was allowed to warm to RT and was stirred for 3 hours. The reaction was loaded directly onto a column and the product purified by F.C.C (eluting with DCM) to yield a pale yellow solid.

To this bromide in DMF (0.1 M) was added Cs₂CO₃ (1.2 eq.) and 4-bromo-3-chlorophenol (1.1 eq.). The reaction was heated to 50° C. for 3 hours, cooled, filtered and evaporated to dryness. The product was purified by column chromatography.

To this bromide in THF (0.1 M) at −78° C. was added triisopropyl borate (1.5 eq.) and then the n-BuLi (1.5 eq.) slowly. The reaction was allowed to stir for an hour and then quenched with NH₄Cl (sat., aq). The reaction was allowed to warm to RT and was diluted with water and EtOAc. The product was extracted with EtOAc (×3). The combined organic layers were washed with brine and dried over MgSO₄. The product was filtered and evaporated to dryness to leave a pale yellow oil.

### Examples 1 to 83

By following the methods described above, the following compounds of formula (I) were prepared.

| | | | | NMR | |
|---|---|---|---|---|---|
| Example | | | Method of | characterisation | |
| No. | Chemical structure | Chemical name | preparation | data | MS |
| 1 | 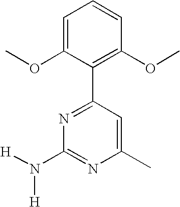 | 4-(2,6- Dimethoxy- phenyl)-6- methyl- pyrimidin-2- ylamine | Method 1A. Starting materials: 2-Amino-4-chloro- 6-methyl- pyrimidine and 2,6- dimethoxyphenyl- boronic acid | ¹H NMR (Me-d₃- OD) 7.36 (1H, t), 6.72 (2H, d), 6.52 (1H, s), 5.75 (6H, s), 2.34 (3H, s) | MS: [M + H]⁺ 246 |
| 2 | 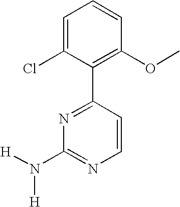 | 4-(2-Chloro-6- methoxy- phenyl)- pyrimidin-2- ylamine | Method 1A. Starting materials: 2-Amino-4- chloropyrimidine and 2-Chloro-6- methoxyphenyl- boronic acid | ¹H NMR (Me-d₃- OD) 8.32 (1H, d), 7.40 (1H, t), 7.12- 7.05 (2H, m), 6.62 (1H, d), 3.78 (3H, s) | MS: [M + H]⁺ 236 |
| 3 | 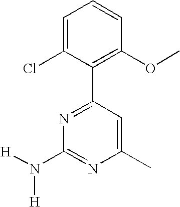 | 4-(2-Chloro-6- methoxy- phenyl)-6- methyl- pyrimidin-2- ylamine | Method 1A. Starting materials: 2-Amino-4- chloro-6-methyl- pyrimidine and 2- Chloro-6- methoxypheny- boronic acid | ¹H NMR (Me-d₃- OD) 7.40 (1H, t), 7.12-7.05 (2H, m), 6.54 (1H, s), 3.78 (3H, s), 2.38 (3H, s) | MS: [M + H]⁺ 250 |
| 4 | 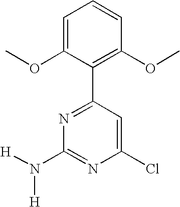 | 4-Chloro-6-(2,6- dimethoxy- phenyl)- pyrimidin-2- ylamine | Method 1A. Starting materials: 2-Amino-4,6- dichloropyrimidine and 2,6- dimethoxyphenyl- boronic acid | ¹H NMR (DMSO- d₆) 7.37 (1H, t), 7.07 (2H, br s), 6.73 (2H, d), 6.53 (1H, s), 3.69 (6H, s) | MS: [M + H]⁺ 266 |
| 5 | 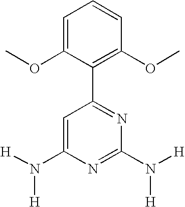 | 6-(2,6- Dimethoxy- phenyl)- pyrimidine-2,4- diamine | Method 1B. Starting materials: 4-Chloro-2,6- diaminopyrimidine and 2,6- dimethoxyphenyl- boronic acid | ¹H NMR (DMSO- d₆) 7.27 (1H, t), 6.66 (2H, d), 6.12 (2H, br s), 5.74 (2H, br s), 5.56 (1H, s), 3.66 (6H, s) | MS: [M + H]⁺ 247 |
| 6 | 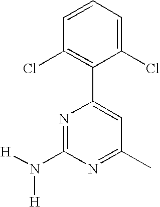 | 4-(2,6-Dichloro- phenyl)-6- methyl- pyrimidin-2-yl- amine | Method 6. Starting materials: 2,6- dichloro- acetophenone, N,N- dimethylacetamide dimethylacetal and guanidine HCl. | ¹H NMR (DMSO- d₆) 7.56 (2H, d), 7.53 (1H, t), 6.69 (2H, s), 6.45 (1H, s), 2.24 (3H, s) | MS: [M + H]⁺ 254 |
| 7 | 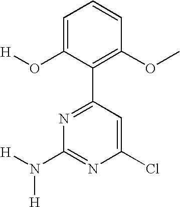 | 2-(2-Amino-6- chloro- pyrimidin-4-yl)- 3-methoxy- phenol | Method 1A, 2A. Starting materials: 2-Amino-4,6- dichloropyrimidine and 2,6- dimethoxyphenyl- boronic acid | ¹H NMR (Me-d₃- OD) 7.56 (1H, s), 7.31-7.26 (1H, m), 6.60-6.55 (2H, m), 3.92 (3H, s) | MS: [M + H]⁺ 252 |
| 8 | 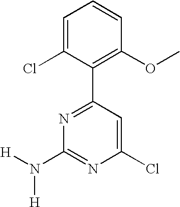 | 4-Chloro-6-(2- chloro-6- methoxy- phenyl)- pyrimidin-2-yl- amine | Method 1A. Starting materials: 2-Amino-4,6- dichloropyrimidine and 2-chloro-6- methoxyphenyl- boronic acid | ¹H NMR (Me-d₃- OD) 7.43-7.39 (1H, m), 7.11-7.07 (2H, m), 6.64 (1H, s), 3.80 (3H, s) | MS: [M + H]⁺ 270 |
| 9 | 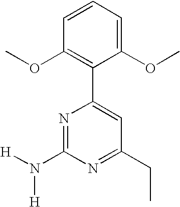 | 4-(2,6- Dimethoxy- phenyl)-6-ethyl- pyrimidin-2-yl- amine | Method 5A, 5B, 1B. Starting material: Ethyl propionylacetate | ¹H NMR (DMSO- d₆) 7.31 (1H, t), 6.70 (2H, d), 6.38 (2H, br s), 6.32 (1H, s), 3.66 (6H, s), 2.56-2.46 (2H, m), 1.17 (3H, t) | MS: [M + H]⁺ 260 |
| 10 | 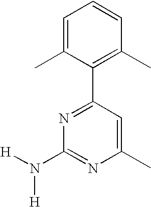 | 4-(2,6- Dimethyl- phenyl)-6- methyl- pyrimidin-2-yl- amine | Method 1E. Starting materials: 2-Amino-4- chloro-6-methyl- pyrimidine and 2,6- dimethylphenyl- boronic acid | ¹H NMR (CDCl₃) 7.20 (1H, t), 7.09 (2H, d), 6.49 (1H, s), 5.48 (2H, s), 2.44 (3H, s), 2.13 (6H, s) | MS: [M + H]⁺ 214 |
| 11 | 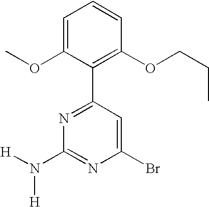 | 4-Bromo-6-(2- methoxy-6- propoxyphenyl)- pyrimidin-2-yl- amine | Method 1A, 2B, 3A. Starting materials: 2- Amino-4,6- dichloropyrimidine and 2,6- dimethoxyphenyl- boronic acid. Alkylation agent: 1-Bromopropane | ¹H NMR (Me-d₃- OD) 7.27 (1H, dd), 6.68 (1H, s), 6.63 (1H, d), 6.61 (1H, d), 3.84 (2H, t), 3.68 (3H, s), 1.61- 1.54 (2H, m), 0.84 (3H, t) | MS: [M + H]⁺ 338 |
| 12 | 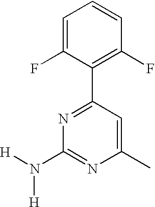 | 4-(2,6-Difluoro- phenyl)-6- methyl- pyrimidin-2-yl- amine | Method 1E. Starting materials: 2-Amino-4-chloro- 6-methyl- pyrimidine and 2,6-difluoropheny- boronic acid | ¹H NMR (CDCl₃) 7.39 (1H, t), 7.03 (2H, d), 6.72 (1H, s), 5.40 (2H, s), 2.48 (3H, s) | MS: [M + H]⁺ 222 |
| 13 | 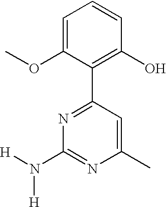 | 2-(2-Amino-6- methyl- pyrimidin-4-yl)- 3-methoxy- phenol | Method 1A, 2B. Starting material: 2-Amino-4- chloro-6-methyl- pyrimidine and 2,6- dimethoxyphenyl- boronic acid | ¹H NMR (CDCl₃) 7.61 (1H, s), 7.28 (1H, t), 6.65 (1H, d), 6.48 (1H, d), 5.60 (2H, s), 3.94 (3H, s), 2.50 (3H, s) | MS: [M + H]⁺ 232 |
| 14 | 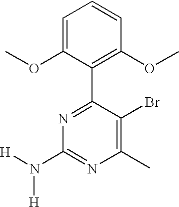 | 5-Bromo-4-(2,6- dimethoxy- phenyl)-6- methyl- pyrimidin-2-yl- amine | Method 1A, 7. Starting material: 2-Amino-4-chloro- 6-methyl- pyrimidine and 2,6- dimethoxyphenyl- boronic acid | ¹H NMR (Me-d₃- OD) 7.35 (1H, t), 6.63 (2H, d), 5.22 (2H, s), 3.79 (6H, s), 2.57 (3H, s) | MS: [M + H]⁺ 323 |
| 15 | 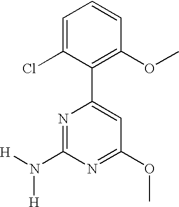 | 4-(2-Chloro-6- methoxy- phenyl)-6- methoxy- pyrimidin-2-yl- amine | Method 1B. Starting materials: 2-Amino-4-chloro- 6-methoxy- pyrimidine and 2- chloro-6- methoxyphenyl- boronic acid | ¹H NMR (Me-d₃- OD) 7.39-7.35 (1H, m), 7.10-7.04 (2H, m), 6.01 (1H, s), 3.95 (3H, s), 3.79 (3H, s) | MS: [M + H]⁺ 266 |
| 16 | 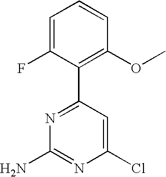 | 4-Chloro-6-(2- fluoro-6- methoxy- phenyl)- pyrimidin-2-yl- amine | Method 1D. Starting materials: 2-Amino-4,6- dichloropyrimidine and (2-fluoro-6- methoxyphenyl)- boronic acid. | ¹H NMR (Me-d₃- OD) 7.35-7.30 (1H, m), 6.85-6.82 (1H, m), 6.74-6.69 (1H, m), 6.61 (1H, s), 3.72 (3H, s) | MS: [M + H]⁺ 254 |
| 17 | 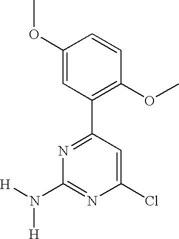 | 4-Chloro-6-(2,5- dimethoxy- phenyl)- pyrimidin-2-yl- amine | Method 1D. Starting materials: 2-Amino-4,6- dichloropyrimidine and 2,5-dimethoxy- phenylboronic acid. | ¹H NMR (Me-d₃- OD) 7.49 (1H, d), 7.25 (1H, s), 7.10- 7.03 (2H, m), 3.87 (3H, s), 3.82 (3H, s) | MS: [M + H]⁺ 266 |
| 18 | 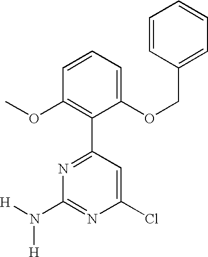 | 4-(2-Benzyloxy- 6-methoxy- phenyl)-6- chloro- pyrimidin-2-yl- amine | Method 1A, 2A, 3B. Starting materials: 2- Amino-4,6- dichloropyrimidine and 2,6-dimethoxy- phenyl-boronic acid. Alkylation agent: Benzyl bromide | ¹H NMR (Me-d₃- OD) 7.39-7.26 (6H, m), 6.81-6.75 (2H, m), 6.64 (1H, s), 5.09 (2H, s), 3.79 (3H, s) | MS: [M + H]⁺ 342 |
| 19 | 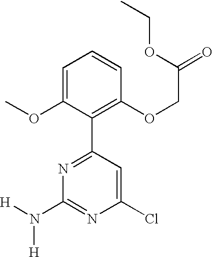 | [2-(2-Amino-6- chloro- pyrimidin-4-yl)- 3-methoxy- phenoxy]-acetic acid ethyl ester | Method 1A, 2A, 3B. Starting materials: 2- Amino-4,6- dichloropyrimidine and 2,6-dimethoxy- phenylboronic acid. Alkylation agent: Ethyl iodoacetate | ¹H NMR (Me-d₃- OD) 7.39-7.34 (1H, m), 6.79 (1H, d), 6.72 (1H, s), 6.63 (1H, d), 4.69 (2H, s), 4.21 (2H, q), 3.80 (3H, s), 1.27 (3H, t) | MS: [M + H]⁺ 338 |
| 20 | 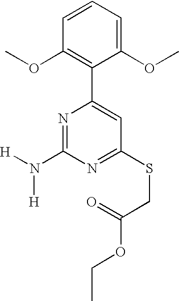 | [2-Amino-6- (2,6-dimethoxy- phenyl)- pyrimidin-4- ylsulfanyl]- acetic acid ethyl ester | Method 1A, 4. Starting materials: 2-Amino-4,6- dichloropyrimidine and 2,6-dimethoxy- phenylboronic acid. Thiol: Ethyl thioglycolate | ¹H NMR (Me-d₃- OD) 7.36 (1H, t), 6.72 (2H, d), 6.49 (1H, s), 4.23 (2H, q), 4.02 (2H, s), 3.76 (6H, s), 1.29 (3H, t) | MS: [M + H]⁺ 350 |
| 21 | 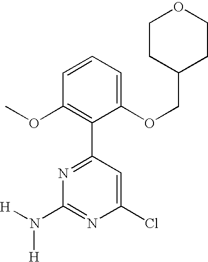 | 4-Chloro-6-[2- methoxy-6- (tetrahydro- pyran-4-yl- methoxy)- phenyl]- pyrimidin-2-yl- amine | Method 1A, 2A, 3B. Starting materials: 2- Amino-4,6- dichloropyrimidine and 2,6- dimethoxyphenyl- boronic acid. Alkylation agent: 4-Bromomethyl- tetrahydropyran | ¹H NMR (Me-d₃- OD) 7.39-7.35 (1H, m), 6.75-6.71 (2H, m), 6.62 (1H, s), 3.95-3.90 (2H, m), 3.85-3.83 (2H, m), 3.79 (3H, s), 3.45-3.37 (2H, m), 1.97-1.89 (1H, m), 1.62-1.56 (2H, m), 1.41-1.29 (2H, m) | MS: [M + H]⁺ 350 |
| 22 | 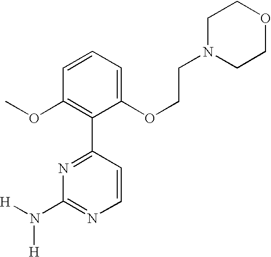 | 4-[2-Methoxy-6- (2-morpholin-4- yl-ethoxy)- phenyl]- pyrimidin-2-yl- amine | Method 1E, 2B, 3C and 9. Starting materials: 4- Chloro-2- methylthio- pyrimidine and 2,6- dimethoxyphenyl- boronic acid. Alkylation agent: 4-(2-Chloroethyl) morpholine. HCl | ¹H NMR (CDCl₃) 8.21 (1H, d), 7.32 (1H, t), 6.60-6.50 (3H, m), 5.15 (2H, s), 4.12 (2H, t), 3.66 (4H, t), 3.62 (3H, s), 2.72 (2H, t), 2.50 (4H, br, s) | MS: [M + H]⁺ 331 |
| 23 | 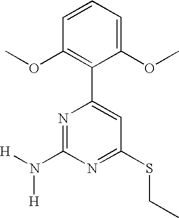 | 4-(2,6- Dimethoxy- phenyl)-6-ethyl- sulphanyl- pyrimidin-2-yl- amine | Method 1A, 4. Starting materials: 2-Amino-4,6- dichloropyrimidine and 2,6- dimethoxyphenyl- boronic acid. Thiol. Ethanethiol | ¹H NMR (Me-d₃- OD) 7.35 (1H, t), 6.70 (2H, d), 6.41 (1H, s), 3.77 (6H, s), 3.14 (2H, q), 1.40 (3H, t) | MS: [M + H]⁺ 292 |
| 24 | 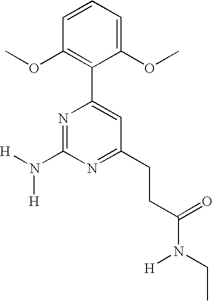 | 3-[2-Amino-6- (2,6-dimethoxy- phenyl)- pyrimidin-4-yl]- N-ethyl- propionamide | Method 5A, 5B, 11. Starting materials: Dimethyl 3- oxoadipate | ¹H NMR (Me-d₃- OD) 7.56 (1H, t), 7.01 (1H, s), 6.86 (2H, d), 3.88 (6H, s), 3.22 (2H, q), 3.12 (2H, t), 2.73 (2H,t), 1.10 (3H, t) | MS: [M + H]⁺ 331 |
| 25 | 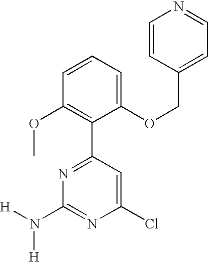 | 4-Chloro-6-[2- methoxy-6- (pyridin-4-yl- methoxy)- phenyl]- pyrimidin-2-yl- amine | Method 1A, 2A, 3D. Starting materials: 2- Amino-4,6- dichloropyrimidine and 2,6- dimethoxyphenyl- boronic acid. Alkylating agent: 4-picolyl chloride hydrochloride | ¹H NMR (Me-d₃- OD) 8.88 (2H, d), 8.08 (2H, d), 7.58 (1H, dd), 7.19 (1H, s), 6.95 (1H, d), 6.89 (1H, d), 5.57 (2H, s), 3.90 (3H, s) | MS: [M + H]⁺ 343 |
| 26 | 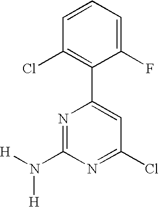 | 4-Chloro-6-(2- chloro-6-fluoro- phenyl)- pyrimidin-2-yl- amine | Method 1A. Starting materials: 2-Amino-4,6- dichloropyrimidine and 2-Chloro-6- fluorophenyl- boronic acid | ¹H NMR (Me-d₃- OD) 7.13-7.09 (1H, m), 6.50-6.47 (2H, m), 6.17 (1H, s) | MS: [M + H]⁺ 258 |
| 27 | 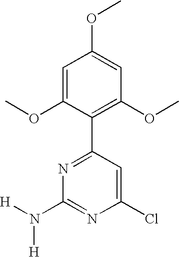 | 4-Chloro-6- (2,4,6- trimethoxy- phenyl)- pyrimidin-2-yl- amine | Method 1A. Starting materials: 2-Amino-4,6- dichloropyrimidine and 2,4,6- trimethoxyphenyl- boronic acid | ¹H NMR (Me-d₃- OD) 6.60 (1H, s), 6.30 (2H, s), 3.87 (3H, s), 3.76 (6H,s) | MS: [M + H]⁺ 296 |
| 28 | 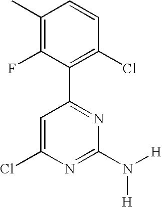 | 4-Chloro-6-(6- chloro-2-fluoro- 3-methyl- phenyl)- pyrimidin-2-yl- amine | Method 1A. Starting materials: 2-Amino-4,6- dichloropyrimidine and 2-Chloro-6- fluoro-5- methylphenyl boronic acid | ¹H NMR (Me-d₃- OD) 7.34 (1H, dd), 7.26 (1H, d), 6.74 (1H, s), 2.31 (3H, ap d) | MS: [M + H]⁺ 272 |
| 29 | 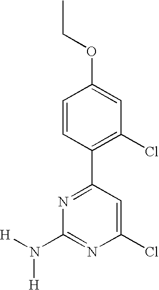 | 4-Chloro-6-(2- chloro-6-ethoxy- phenyl)- pyrimidin-2-yl- amine | Method 1A (reaction length 5 hours). Starting materials: 2-amino-4,6- dichloropyrimidine and (2-chloro-4- ethoxy)- phenylboronic acid | ¹H NMR (Me-d₃- OD) 7.56 (1H, d), 7.08 (1H, d), 6.99 (1H, dd), 6.94 (1H, s), 4.12 (2H, q), 1.43 (3H, t) | MS: [M + H]⁺ 284 |
| 30 | 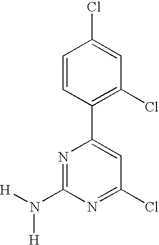 | 4-Chloro-6-(2,4- dichloro- phenyl)- pyrimidin-2-yl- amine | Method 1A (reaction length 5 hours). Starting material: 2-amino-4,6- dichloropyrimidine and 2,4- dichlorophenyl- boronic acid | ¹H NMR (Me-d₃- OD) 7.61 (1H, d), 7.59 (1H, d), (1H, dd), 6.93 (1H, s) | MS: [M + H]⁺ 274 |
| 31 | 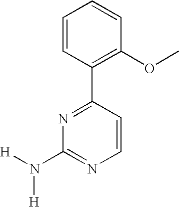 | 4-(2-Methoxy- phenyl)- pyrimidin-2-yl- amine | Method 1A. Starting materials: 2-Amino-4- chloropyrimidine and 2- methylphenyl- boronic acid | ¹H NMR (Me-d₃- OD) 8.11 (1H, d), 7.64 (1H, d), 7.32 (1H, t), 7.02 (1H, d), 7.01 (1H, d), 6.93 (1H, t), 3.76 (3H, s) | MS: [M + H]⁺ 202 |
| 32 | 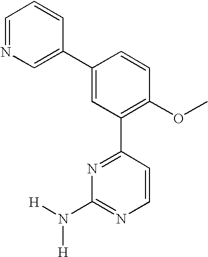 | 4-(2-Methoxy-5- pyridin-3-yl- phenyl) pyrimidin-2-yl- amine | Method 1A. Starting materials: 4-(5-Bromo-2- methoxy-phenyl)- pyrimidin-2-yl- amine and 5- (pyridin-3-yl)- boronic acid | ¹H NMR (Me-d₃- OD) 8.83 (1H, s), 8.51 (1H, d), 8.26(1H, d), 8.15- 8.10 (2H, m), 7.88 (1H, dd), 7.52 (1H, dd), 7.32-7.25 (2H, m), 3.97 (3H, s) | MS: [M + H]⁺ 279 |
| 33 | 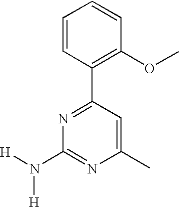 | 4-(2-Methoxy- phenyl)-6- methyl- pyrimidin-2-yl- amine | Method 1A. Starting materials: 2-Amino-4-chloro- 6-methyl- pyrimidine and 2- Methoxy phenylboronic acid | ¹H NMR (Me-d₃- OD) 7.68 (1H, d), 7.43 (1H, t), 7.12 (1H, d), 7.05 (1H, t), 7.03 (1H, s), 3.88 (3H, s), 2.36 (3H, s) | MS: [M + H]⁺ 216 |
| 34 | 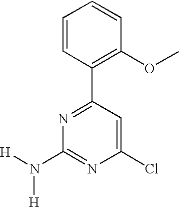 | 4-Chloro-6-(2- methoxy- phenyl)- pyrimidin-2-yl- amine | Method 1A. Starting materials: 2-Amino-4,6- dichloropyrimidine and 2-Methoxy phenylboronic acid | ¹H NMR (Me-d₃- OD) 7.83 (1H, d), 7.45 (1H, t), 7.20 (1H, s), 7.12 (1H, d), 7.05 (1H, t), 3.92 (3H, s) | MS: [M + H]⁺ 236 |
| 35 | 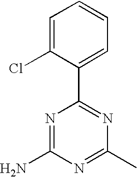 | 4-(2-Chloro- phenyl)-6- methyl- [1,3,5]triazin-2- yl-amine | Method 6. Starting materials: 2- Chlorobenzamide, N,N- dimethylacetamide dimethylacetal and guanidine HCl. | ¹H NMR (CDCl₃) 7.70 (1H, d), 7.50 (1H, d), 7.39 (2H, t), 5.05 (2H, s), 2.55 (3H, s) | MS: [M + H]⁺ 221 |
| 36 | 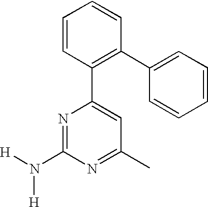 | 4-Biphenyl-2-yl- 6-methyl- pyrimidin-2- ylamine | Method 1E. Starting materials: 2-Amino-4-chloro- 6-methyl- pyrimidine and 2- biphenyl boronic acid. | ¹H NMR (CDCl₃) 7.68 (1H, d), 7.53- 7.42 (3H, m), 7.33- 7.27 (3H, m), 7.23- 7.21 (2H, m), 6.12 (1H, s), 5.33 (2H, s), 2.16 (3H, s) | MS: [M + H]⁺ 262 |
| 37 | 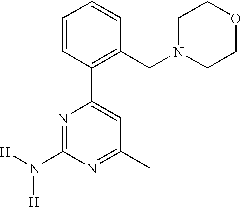 | 4-Methyl-6-(2- morpholin-4-yl- methyl-phenyl)- pyrimidin-2- ylamine | Method 8, 1E. Starting materials: (2-bromomethyl- phenyl)boronic acid, morpholine and 2-amino-4- chloro-6- methylpyrimidine. | ¹H NMR (Me-d₃- OD) 7.46 (1H, d), 7.42-7.36 (3H, m), 6.75 (1H, s), 3.63 (2H, s), 3.50 (4H, t), 2.40 (3H, s), 2.30 (4H, br, s) | MS: [M + H]⁺ 285 |
| 38 | 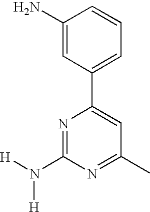 | 4-(3-Amino- phenyl)-6- methyl- pyrimidin-2-yl- amine | Method 1B. Starting materials: 2-Amino-4-chloro- 6-methyl- pyrimidine and 3- aminobenzene boronic acid. | ¹H NMR (CDCl₃) 7.23 (1H, s), 7.15- 7.06 (2H, m), 6.84 (1H, s), 6.65 (1H, d), 6.42 (2H, s), 5.17 (2H, s), 2.26 (3H, s) | MS: [M + H]⁺ 201 |
| 39 | 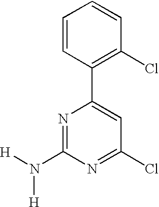 | 4-Chloro-6-(2- chloro-phenyl)- pyrimidin-2-yl- amine | Method 1C. Starting materials: 2-amino-4,6- dichloropyrimidine and 2- chlorophenyl- boronic acid. | ¹H NMR (Me-d₃- OD) 7.47-7.41 (2H, m), 7.37-7.29 (2H, m), 6.79 (1H, s) | MS: [M + H]⁺ 240 |
| 40 | 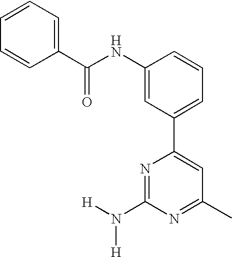 | N-[3-(2-Amino- 6-methyl- pyrimidin-4-yl)- phenyl]- benzamide | Method 10. Starting materials: 4-(3-amino- phenyl)-6-methyl- pyrimidin-2- ylamine and benzoyl chloride. | ¹H NMR (Me-d₃- OD) 8.40 (1H, s), (2H, d) 7.84 (2H, d), 7.62-7.45 (4H, m), 7.05 (1H, s), 2.40 (3H, s) | MS: [M + H]⁺ 305 |
| 41 | 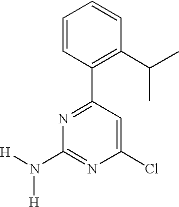 | 4-Chloro-6-(2- isopropyl- phenyl)- pyrimidin-2-yl- amine | Method 1A. Starting materials: 2-amino-4,6- dichloropyrimidine and 2-isopropyl- phenylboronic acid | ¹H NMR (Me-d₃- OD) 7.47-7.41 (2H, m), 7.28-7.23 (2H, m), 6.71 (1H, s), 3.20 (1H, sp), 1.23 (6H, d) | MS: [M + H]⁺ 248 |
| 42 | 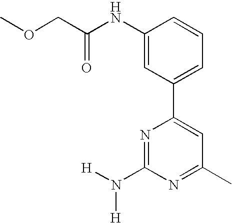 | N-[3-(2-Amino- 6-methyl- pyrimidin-4-yl)- phenyl]-2- methoxy- acetamide | Method 10. Starting materials: 4-(3-amino- phenyl)-6-methyl- pyrimidin-2- ylamine and methoxyacetyl chloride. | ¹H NMR (Me-d₃- OD) 8.30 (1H, s), 8.13 (1H, s), 7.81 (1H, d), 7.74 (1H, d), 7.45 (1H, t), 7.05 (1H, s), 4.08 (2H, s), 3.52 (3H, s), 2.40 (3H, s) | MS: [M + H]⁺ 273 |
| 43 | 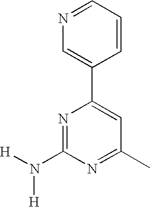 | 4-Methyl-6- pyridin-3-yl- pyrimidin-2-yl- amine | Method 1A. Starting materials: 2-amino-4-chloro- 6-methyl- pyrimidine and pyridine-3-boronic acid | ¹H NMR (Me-d₃- OD) 9.20 (1H, s), 8.65 (1H, d), 8.46 (1H, d), 7.55 (1H, dd), 7.12 (1H, s), 2.41 (3H, s) | MS: [M + H]⁺ 187 |
| 44 | 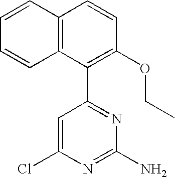 | 4-Chloro-6-(2- ethoxy- naphthalen-1- yl)-pyrimidin-2- ylamine | Method 1A. Starting materials: 2-Amino-4,6- dichloropyrimidine and 2-Ethoxy-1- naphthaleneboronic acid | ¹H NMR (Me-d₃- OD) 7.99 (1H, d), 7.87 (1H, d), 7.54- 7.34 (4H, m), 6.78 (1H, s), 4.21 (2H, q), 1.34 (3H, t) | MS: [M + H]⁺ 300 |
| 45 | 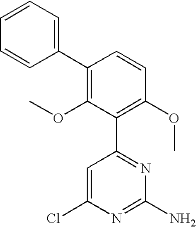 | 4-Chloro-6-(2,4- dimethoxy- biphenyl- 3-yl)-pyrimidin- 2-ylamine | Step 1: Method 12A. Starting materials: 2- Dimethoxyphenyl- boronic acid. Step 2: Method 1A: 2- Amino-2,6- dichloropyrimindine. Step 3: Method 1F: Startings materials: Phenylboronic acid | ¹H NMR (Me-d₃- OD) 7.57-7.52 (2H, m), 7.45-7.39 (3H, m), 7.34 (1H, t), 6.97 (1H, d), 6.73 (1H, s), 3.83 (3H, s), 3.26 (3H, s) | MS: [M + H]⁺ 342 |
| 46 | 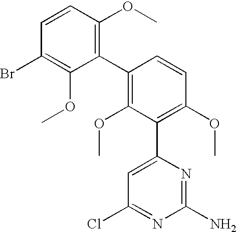 | 4-(3′-Bromo- 2,4,2′,6′- tetramethoxy- biphenyl-3-yl)- 6-chloro- pyrimidin-2- ylamine | Step 1: Method 12A. Starting materials: 2- Dimethoxyphenyl- boronic acid. Step 2: Method 1A: 2- Amino-2,6- dichloropyrimindine. | ¹H NMR (Me-d₃- OD) 7.57 (1H, d), 7.21 (1H, d), 6.94 (1H, d), 6.82 (1H, d), 6.72 (1H, s), 3.82 (3H, s), 3.75 (3H, s), 3.57 (3H, s), 3.32 (3H, s) | MS: [M + H]⁺ 480 |
| 47 | 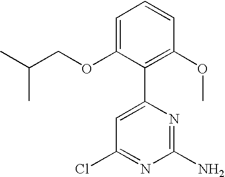 | 4-Chloro-6-(2- isobutoxy-6- methoxy- phenyl)- pyrimidin-2- ylamine | Method 1A, 2B, 3D (90° C.). Starting materials: 2-Amino-4,6- dichloropyrimidine and 2,6- dimethoxyphenyl- boronic acid. Alkylation agent: 1-Bromo-2- methylpropane. HCl salt. | ¹H NMR (Me-d₃- OD) 7.56 (1H, t), 7.17 (1H, s), 6.84 (1H, d), 6.82 (1H, d), 3.90 (3H, s), 3.85 (2H, d), 2.04 (1H, m), 0.98 (6H, d) | MS: [M + H]⁺ 308 |
| 48 | 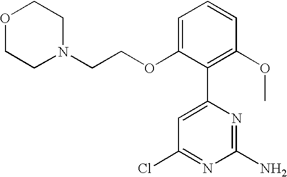 | 4-Chloro-6-[2- methoxy-6-(2- morpholin- 4-yl-ethoxy)- phenyl]- pyrimidin-2- ylamine | Method 1A, 2B, 3D (90° C.). Starting materials: 2-Amino-4,6- dichloropyrimidine and 2,6- dimethoxyphenyl- boronic acid. Alkylation agent: 1-Bromo-2- methylpropane. HCl salt. | ¹H NMR (Me-d₃- OD) 7.57 (1H, t), 7.07 (1H, s), 6.93 (1H, d), 6.88 (1H, d), 4.50 (2H, t), 3.96 (4H, br s), 3.87 (3H, s), 3.62 (2H, t), 3.36 (4H, br s) | MS: [M + H]⁺ 365 |
| 49 | 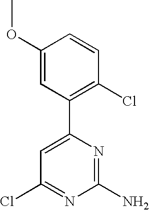 | 4-Chloro-6-(2- chloro-5- methoxy- phenyl)- pyrimidin-2- ylamine | Method 1A (80° C,, 3 hours). Starting materials: 2- Amino-4,6- dichloropyrimidine and (2-chloro-5- methoxyphenyl) boronic acid. | ¹H NMR (Me-d₃- OD) 7.42 (1H, d), 7.13 (1H, d), 7.04 (1H, dd), 6.92 (1H, s), 3.84 (3H, s) | MS: [M + H]⁺ 270 |
| 50 | 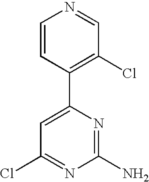 | 4-Chloro-6-(3- chloro-pyridin- 4-yl)- pyrimidin-2- ylamine | Method 1A (80° C., 3 hours). Starting materials: 2- Amino-4,6- dichloropyrimidine and 3-Chloro-4- (5,5-dimethyl- 1,3,2- dioxaborinan-2- yl)pyridine. | ¹H NMR (Me-d₃- OD) 8.71 (1H, s), 8.60 (1H, d), 7.64 (1H, d), 7.00 (1H, s) | MS: [M + H]⁺ 241 |
| 51 | 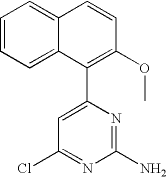 | 4-Chloro-6-(2- methoxy- naphthalen-1- yl)-pyrimidin-2- ylamine | Method 1A. Starting materials: 2-Amino-4,6- dichloropyrimidine and 2-Methoxy-1- naphthaleneboronic acid. | ¹H NMR (Me-d₃- OD) 8.02 (1H, d), 7.88 (1H, d), 7.54- 7.35 (4H, m), 6.77 (1H, s), 3.93 (3H, s) | MS: [M + H]⁺ 286 |
| 52 | 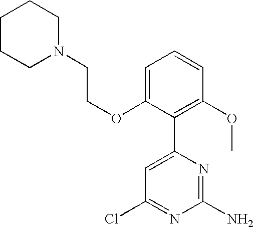 | 4-Chloro-6-[2- methoxy-6-(2- piperidin-1-yl- ethoxy)-phenyl]- pyrimidin-2- ylamine | Method 1A, 2B, 3D. Starting materials: 2- Amino-4,6- dichloropyrimidine and 2,6- dimethoxyphenyl- boronic acid. Alkylation agent: 1-(2- Chloroethly) piperidine HCl salt. Formate salt. | ¹H NMR (Me-d₃- OD) 8.20 (1H, br s), 7.43 (1H, dd), 6.83 (1H, d), 6.78 (1H, d), 5.72 (1H, s), 4.42 (2H, dd), 3.83 (3H, s), 3.51 (2H, dd), 3.34-3.24 (4H, m), 1.89-1.70 (4H, m), 1.71-1.60 (2H, m) | MS: [M + H]⁺ 363 |
| 53 | 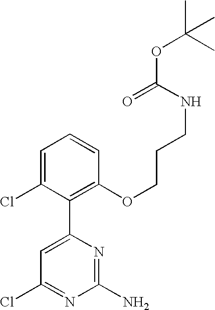 | {3-[2-(2-Amino- 6-chloro- pyrimidin-4- yl)-3-chloro- phenoxy]- propyl}- carbamic acid tert-butyl ester | Method 1A, 2B, 3D (40° C.). Starting materials: 2-Amino-4,6- dichloropyrimidine and 2-Chloro-6- methoxyphenyl- boronic acid. Alkylation agent: 3-(BOC- amino)propyl bromide. | ¹H NMR (Me-d₃- OD) 7.27 (1H, dd), 6.99 (1H, d), 6.95 (1H, dd), 6.59 (1H, s), 3.93 (2H, t), 2.94 (2H, t), 1.75- 1.66 (2H, m), 1.31 (9H, s) | MS: [M + H]⁺ 413 |
| 54 | 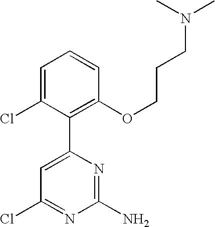 | 4-Chloro-6-[2- chloro-6-(3- dimethylamino- propoxy)- phenyl[- pyrimidin-2-yl- amine | Method 1A, 2B, 3D (50° C.). Starting materials: 2-Amino-4,6- dichloropyrimidine and 2-Chloro-6- methoxyphenyl- boronic acid. Alkylation agent: 3-Dimethylamino- propyl chloride. | ¹H NMR (Me-d₃- OD) 7.40 (1H, dd), 7.12 (1H, d), 7.06 (1H, d), 6.67 (1H, s), 4.08-4.04 (2H, m), 2.49-2.44 (2H, m), 2.31 (6H, s), 1.91-1.85 (2H, m) | MS: [M + H]⁺ 341 |
| 55 | 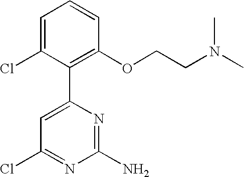 | 4-Chloro-6-[2- chloro-6-(2- dimethylamino- ethoxy)-phenyl]- pyrimidin-2-yl amine | Method 1A, 2B, 3D (50° C.). Starting materials: 2-Amino-4,6- dichloropyrimidine and 2-Chloro-6- methoxyphenyl- boronic acid. Alkylation agent: 3-Dimethylamino- ethyl chloride. | ¹H NMR (Me-d₃- OD) 7.40 (1H, dd), 7.13 (1H, d), 7.08 (1H, d), 6.68 (1H, s), 4.14 (2H, t), 2.68 (2H, t), 2.24 (6H, s) | MS: [M + H]⁺ 327 |
| 56 | 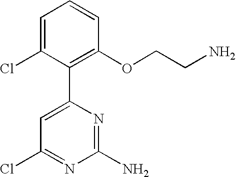 | 4-[2-(2-Amino- ethoxy)-6- chloro-phenyl]- 6-chloro- pyrimidin-2-yl- amine | Method 1A, 2B, 3D (50° C.). Starting materials: 2-Amino-4,6- dichloropyrimidine and 2-Chloro-6- methoxyphenyl- boronic acid. Alkylation agent: 2-(BOC- amino)ethyl bromide. Step 4; HCl/ether. | ¹H NMR (Me-d₃- OD) 7.42 (1H, dd), 7.15 (1H, d), 7.10 (1H, d), 6.70 (1H, s), 4.11 (2H, t), 2.94 (2H, t) | MS: [M + H]⁺ 299 |
| 57 | 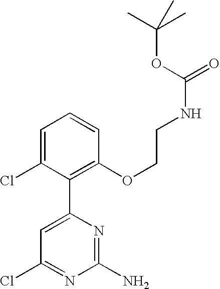 | {2-[2-(2-Amino- 6-chloro- pyrimidin-4-yl)- 3-chloro- phenoxy]- acid tert-butyl ester | Method 1A, 2B, 3D (50° C.). Starting materials: 2-Amino-4,6- dichloropyrimidine and 2-Chloro-6- methoxyphenyl- boronic acid. Alkylation agent: 2-(BOC- amino)ethyl bromide. | ¹H NMR (Me-d₃- OD) 7.28 (1H, t), 7.03-7.96 (2H, m), 6.57 (1H, s), 3.97 (2H, t), 3.25-3.17 (2H, m), 1.33 (9H, s) | MS: [M + H]⁺ 399 |
| 58 | 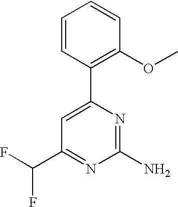 | 4-Difluoro- methyl-6-(2- methoxy-phenyl)- pyrimidin-2- ylamine | Method 5C. Starting materials: 4,4-Difluoro-1-(2- methoxy-phenyl)- butane-1,3-dione. | ¹H NMR (Me-d₃- OD) 7.86 (1H, dd), 7.51-7.46 (1H, m), 7.40 (1H, s), 7.16 (1H, d), 7.08 (1H, ddd), 6.50 (1H, t), 3.92 (3H, s) | MS: [M + H]⁺ 252 |
| 59 | 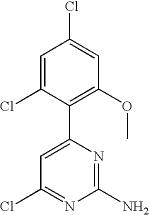 | 4-Chloro-6-(2,4- dichloro-6- methoxy- phenyl)- pyrimidin-2- ylamine | Method 13A, 1A. Starting materials: 3,5- Dichloroanisole. 2- Amino-4,6- dichloropyrimidine | ¹H NMR (Me-d₃- OD) 7.18 (1H, d), 7.14 (1H, d), 6.65 (1H, s), 3.82 (3H, s) | MS: [M + H]⁺ 304 |
| 60 | 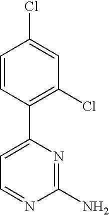 | 4-(2,4-Dichloro- phenyl)- pyrimidin-2- ylamine | Method 14. Starting materials: 1-(2,4- Dichlorophenyl)-3- (dimethylamino)-2- propene-1-one. | ¹H NMR (Me-d₃- OD) 8.22 (1H, d), 7.50 (1H, d), 7.47 (1H, d), 7.35 (1H, dd), 6.81 (1H, d) | MS: [M + H]⁺ 240 |
| 61 | 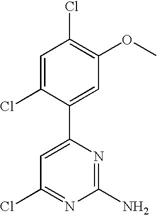 | 4-Chloro-6-(2,4- dichloro-5- methoxy- phenyl)- pyrimidin-2- ylamine | Method 12B, 1E. Starting materials: (2-Chloro-5- Methoxyphenyl) boronic acid. Amino- 4,6- dichloropyrimidine | ¹H NMR (Me-d₃- OD) 7.57 (1H, s), 7.29 (1H, s), 6.97 (1H, s), 3.94 (3H, s) | MS: [M + H]⁺ 304 |
| 62 | 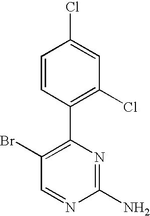 | 5-Bromo-4-(2,4- dichloro- phenyl)- pyrimidin-2- ylamine | Method 14, 12A. Starting materials: 1-(2,4- Dichlorophenyl)-3- (dimethylamino)-2- propene-1-one. | ¹H NMR (Me-d₃- OD) 8.44 (1H, s), 7.61 (1H, d), 7.48 dd), 7.35 (1H, d) | MS: [M + H]⁺ 318 |
| 63 | 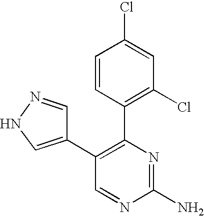 | 4-(2,4-Dichloro- phenyl)-5-(1H- pyrazol-4-yl)- pyrimidin-2- ylamine | Method 14, 12A, 1F. Starting materials: 1-(2,4- Dichlorophenyl)-3- (dimethylamino)-2- propene-1-one. | ¹H NMR (Me-d₃- OD) 8.54 (1H, s), 7.57 (1H, d), 7.49 (1H, dd), 7.41 (1H, d), 7.31 (1H, s), 5.50 (1H, s) | MS: [M + H]⁺ 306 |
| 64 | 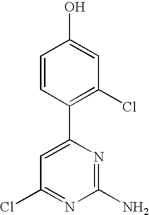 | 4-(2-Amino-6- chloro- pyrimidin-4-yl)- 3-chloro-phenol | Method 1A, 2C (50° C.). Starting materials: 2- Amino-4,6- dichloropyrimidine and 2-Chloro-4- ethoxyphenyl- boronic acid. | ¹H NMR (Me-d₃- OD) 7.49 (1H, d), 6.94-6.92 (1H, m), 6.84 (1H, dd) | MS: [M + H]⁺ 256 |
| 65 | 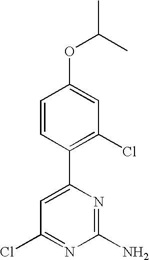 | 4-Chloro-6-(2- chloro-4- isopropoxy- phenyl)- pyrimidin-2- ylamine | Method 1A, 2C, 3D (50° C.). Starting materials: 2-Amino-4,6- dichloropyrimidine and 2-Chloro-4- ethoxyphenyl- boronic acid. Alkylation agent: 2-Iodopropane. | ¹H NMR (Me-d₃- OD) 7.55 (1H, d), 7.05 (1H, d), 6.98 (1H, d), 6.95 (1H, s), 4.70 (1H, sept), 1.36 (6H, d) | MS: [M + H]⁺ 298 |
| 66 | 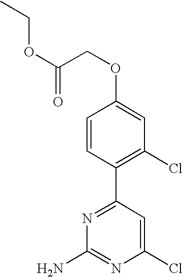 | [4-(2-Amino-6- chloropyrimidin- 4-yl)-3-chloro- phenoxy]-acetic acid ethyl ester | Method 1A, 2C, 3D (50° C.). Starting materials: 2-Amino-4,6- dichloropyrimidine and 2-Chloro-4- ethoxyphenyl- boronic acid. Alkylation agent: Ethyl bromoacetate. | ¹H NMR (Me-d₃- OD) 7.58 (1H, d), 7.12 (1H, d), 7.02 (1H, dd), 6.94 (1H, s), 4.80 (2H, s), 4.28 (2H, q), 1.31 (3H, t) | MS: [M + H]⁺ 342 |
| 67 | 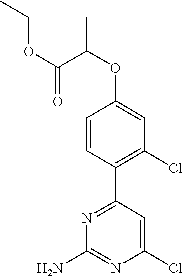 | 2-[4-(2-Amino- 6-chloro- pyrimidin-4- yl)-3-chloro- phenoxy]- ethoxyphenyl- propionic acid ethyl ester | Method 1A, 2C, 3D (50° C.). Starting materials: 2-Amino-4,6- dichloropyrimidine and 2-Chloro-4- ethoxyphenyl- boronic acid. Alkylation agent: Ethyl 2- bromopropionate. | ¹NMR (Me-d₃- OD) 7.56 (1H, d), 7.06 (1H, d), 6.97 (1H, dd), 6.93 (1H, s), 4.99 (1H, q), 4.24 (2H, q), 1.62 (3H, d), 1.28 (3H, t) | MS: [M + H]⁺ 356 |
| 68 | 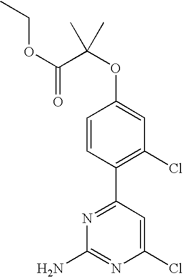 | 2-[4-(2-Amino- 6-chloro- pyrimidin-4- yl)-3-chloro- phenoxy]-2- methyl- propionic acid ethyl ester | Method 1A, 2C, 3D (50° C.). Starting materials: 2-Amino-4,6- dichloropyrimidine and 2-Chloro-4- ethoxyphenyl- boronic acid. Alkylation agent: Ethyl 2- bromoisobutyrate. | ¹H NMR (Me-d₃- OD) 7.53 (1H, d), 6.98 (1H, d), 6.93 (1H, s), 6.88 (1H, dd), 4.25 (2H, q), 1.65 (6H, s), 1.26 (3H, t) | MS: [M + H]⁺ 370 |
| 69 | 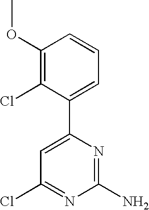 | 4-Chloro-6-(2- chloro-3- methoxy- phenyl)- pyrimidin-2-yl- amine | Method 13B, 1E (thermally). Starting materials: 2-Chloroanisole. 2- Amino-4,6-dichloropyrimidine | ¹H NMR (Me-d₃- OD) 7.39 (1H, dd), 7.20 (1H, dd), 7.10 (1H, dd), 6.85 (1H, s), 3.95 (3H, s) | MS: [M + H]⁺ 270 |
| 70 | 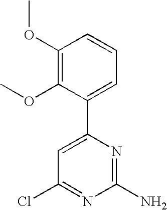 | 4-Chloro-6-(2,3- dimethoxy- phenyl)- pyrimidin-2-yl- amine | Method 1E (thermally). Starting materials: 2-Amino-4,6- dichloropyrimidine and 2,3- Dimethoxyphenyl- boronic acid. | ¹H NMR (Me-d₃- OD) 7.33 (1H, dd), 7.18-7.16 (2H, m), 7.11 (1H, s), 3.92 (3H, s), 3.78 (3H, s) | MS: [M + H]⁺ 266 |
| 71 | 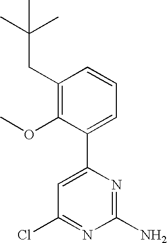 | 4-Chloro-6-[3- (2,2-dimethyl- propyl)-2- methoxy- phenyl]- pyrimidin-2-yl- amine | Method 15, 13C, 1F (50° C., 2 hours). Starting materials: 2- Chlorobenzyl bromide. | ¹H NMR (Me-d₃- OD) 7.51 (1H, dd), 7.22 (1H, dd), 7.06 (1H, dd), 7.05 (1H, s), 3.44 (3H, s), 2.55 (2H, 5), 0.88 (9H, s) | MS: [M + H]⁺ 306 |
| 72 | 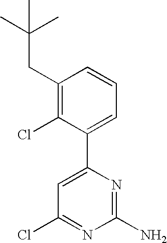 | 4-Chloro-6-[2- chloro-3-(2,2- dimethyl- propyl)phenyl]- pyrimidin-2-yl- amine | Method 15, 13B, 1F (50° C., 2 hours). Starting materials: 2- Methoxybenzyl chloride. | ¹H NMR (Me-d₃- OD) 7.40 (1H, dd), 7.36-7.33 (2H, m), 6.81 (1H, s), 2.85 (2H, s), 1.01 (9H, s) | MS: [M + H]⁺ 310 |
| 73 | 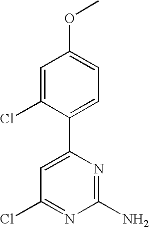 | 4-Chloro-6-(2- chloro-4- methoxy- phenyl)- pyrimidin-2- ylamine | Method 1G. Starting materials: 2-Amino-4,6- dichloropyrimidine and (2- Chloromethoxy- phenyl)boronic acid. | ¹H NMR (Me-d₃- OD) 7.57 (1H, d), 7.09 (1H, d), 7.01 (1H, dd), 6.95 (1H, s), 3.88 (3H, s) | MS: [M + H]⁺ 270 |
| 74 | 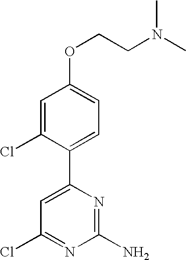 | 4-Chloro-6-[2- chloro-4-(2- dimethylamino- ethoxy)-phenyl]- pyrimidin-2-yl amine | Method 1A, 2C, 3D (50° C.). Starting materials: 2-Amino-4,6- dichloropyrimidine and 2-Chloro-4- ethoxyphenyl- boronic acid. Alkylation agent: 2-Dimethylamino- ethyl chloride HCl. | ¹H NMR (Me-d₃- OD) 7.49 (1H, d), 7.05 (1H, d), 6.95 (1H, dd), 6.85 (1H, s), 4.11 (2H, t), 2.76 (2H, t), 2.31 (6H, s) | MS: [M + H]⁺ 327 |
| 75 | 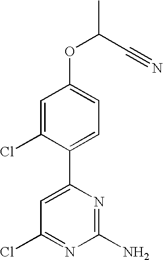 | 2-[4-(2-Amino- 6-chloro- pyrimidin-4- yl)-3-chloro- phenoxy]- propionitrile | Method 1A, 2C, 3D (50° C.). Starting materials: 2-Amino-4,6- dichloropyrimidine and 2-Chloro-4- ethoxyphenyl- boronic acid. Alkylation agent: Bromopropionitrile | ¹H NMR (Me-d₃- OD) 7.64 (1H, dd), 7.28 (1H, d), 7.15 (1H, dd), 6.96 (1H, s), 5.36 (1H, q), 1.79 (3H, d) | MS: [M + H]⁺ 309 |
| 76 | 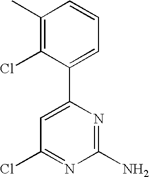 | 4-Chloro-6-(2- chloro-3-methyl- phenyl)- pyrimidin-2-yl- amine | Method 1H. Starting materials: 2-Amino-4,6- dichloropyrimidine and 2-Chloro-3- methylphenyl- boronic acid | ¹H NMR (Me-d₃- OD) 7.42 (1H, dd), 7.34-7.30 (2H, m), 6.85 (1H, s), 2.46 (3H, s) | MS: [M + H]⁺ 254 |
| 77 | 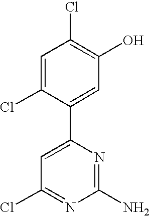 | 5-(2-Amino-6- chloro- pyrimidin-4-yl)- 2,4-dichloro- phenol | Method 16, 2A: Starting materials: 5-(benzyloxy)-2,4- dichloroaniline. | ¹H NMR (Me-d₃- OD) 7.48 (1H, s), 7.11 (1H, s), 6.90 (1H, s) | MS: [M + H]⁺ 290 |
| 78 | 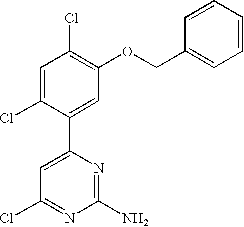 | 4-(5-Benzyloxy- 2,4-dichloro- phenyl)- 6-chloro- pyrimidin-2-yl- amine | Method 16: Starting materials: 5-(benzyloxy)-2,4- dichloroaniline. | ¹H NMR (Me-d₃- OD) 7.59 (1H, s), 7.51-7.48 (2H, m), 7.44-7.33 (4H, m), 6.93 (1H, s), 5.23 (2H, s) | MS: [M + H]⁺ 380 |
| 79 | 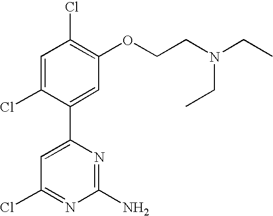 | 4-Chloro-6-[2,4- dichloro-5-(2- diethylamino- ethoxy)-phenyl]- pyrimidin-2-yl- amine | Method 16, 2A, 3C: Starting materials: 5- (benzyloxy)-2,4- dichloroaniline. Alkylation agent: 2-(Diethylamino) ethylbromide HBr. | ¹H NMR (Me-d₃- OD) 7.58 (1H, s), 7.32 (1H, s), 6.97 (1H, s), 4.21 (2H, t), 3.01 (2H, t), 2.73 (4H, q), 1.13 (6H, t) | MS: [M + H]⁺ 389 |
| 80 | 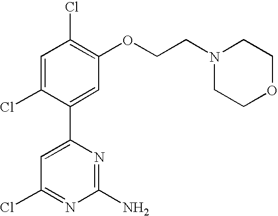 | 4-Chloro-6-[2,4- dichloro-5-(2- morpholin-4-yl- ethoxy)-phenyl]- pyrimidin-2-yl- amine | Method 16, 2A, 3C: Starting materials: 5- (benzyloxy)-2,4- dichloroaniline. Alkylation agent: N-(2- Chloroethyl) morpholine HCl. | ¹H NMR (Me-d₃- OD) 7.58 (1H, s), 7.33 (1H, s), 6.96 (1H, s), 4.27 (2H, t), 3.72 (4H, dd), 2.88 (2H, t), 2.66 (4H, dd) | MS: [M + H]⁺ 403 |
| 81 | 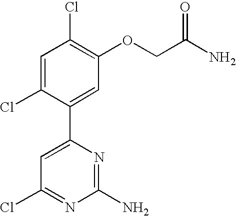 | 2-[5-(2-Amino- 6-chloro- pyrimidin-4-yl)- 2,4-dichloro- phenoxy]- acetamide | Method 16, 2A, 3C: Starting materials: 5- (benzyloxy)-2,4- dichloroaniline. Alkylation agent: 2-Bromoacetamide | ¹H NMR (Me-d₃- OD) 7.64 (1H, s), 7.32 (1H, s), 6.97 (1H, s), 4.65 (2H, s) | MS: [M + H]⁺ 347 |
| 82 | 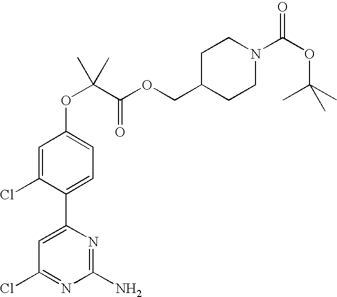 | 4-{2-[4-(2- Amino-6-chloro- pyrimidin-4-yl)- 3-chloro- phenoxy]-2- methyl- propionyloxy- methyl}- piperidine-1- carboxylic acid tert-butyl ester | Method 17, 1H. Starting materials: 2-bromoisobutyryl bromide. | ¹H NMR (Me-d₃- OD) 7.55 (1H, d), 6.97 (1H, d), 6.93 (1H, s), 6.89 (1H, dd), 4.07-4.00 (4H, m), 2.77- 2.65 (2H, m), 1.86-1.76 (1H, m), 1.67 (6H,s), 1.59-1.52 (2H, m), 1.45 (9H, s), 1.11-1.05 (2H, m) | MS: [M + H]⁺ 539 |
| 83 | 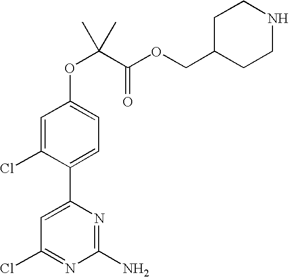 | 2-[4-(2-Amino- 6-chloro- pyrimidin-4- yl)-3-chloro- phenoxy]-2- methyl-prop- ionic acid piperidin-4- ylmethyl ester | Method 17, 1H, HCl/ether. Starting materials: 2- bromoisobutyryl bromide. | ¹H NMR (Me-d₃- OD) 7.45 (1H, d), 6.89 (1H, s) 6.88 (1H, d), 6.80 (1H, dd), 4.01 (1H, d), 3.98 (1H, d), 3.30- 3.21 (2H, m), 2.92- 2.79 (2H, m), 1.93- 1.82 (1H, m), 1.74- 1.66 (2H, m), 1.57 (6H, s), 1.38-1.28 (2H, m) | MS: [M + H]⁺ 439 |

The following compounds are commercially available.

| Example | | | | |
|---|---|---|---|---|
| No. | | | | |
| AT | | Chemical | | Catalogue |
| number | Chemical structure | name | Supplier | Number |
| 84 | 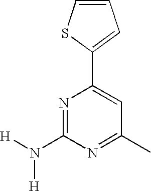 | 4-(2-thienyl)-2- pyrimidinamine | BIONET-INTER | 8R-0605 |
| 85 | 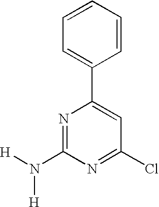 | 4-chloro-6- phenyl- pyrimidin-2- ylamine | ASINEX | BAS 0489257 |
| 86 | 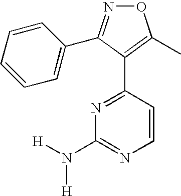 | 4-(3-phenyl-5- methyl- isoxazol-4-yl)- 2-pyrimidin- amine | MAYBRIDGE | SPB 05204 |

### Biological Activity

### Example 87

### Isothermal Titration Calorimetry

The ability of the compounds of the invention to bind to human Hsp90 proteins was determined using isothermal titration calorimetry.

Isothermal titration calorimetry (ITC) experiments were performed with a VP-ITC titration calorimeter (Microcal Inc., Northampton, Mass., USA). Cloning, expression, and purification of the Human Hsp90α N-terminal domain were performed according to published methods (Jez, J. M. et al, Chem. Biol. 2003 April; 10(4):361-8.) Solutions of the human Hsp90α N-terminal domain and compound were prepared in a buffer comprising 25 mM Tris, 100 mM NaCl, 1 mM MgCl₂, 1 mM TCEP, 5% DMSO, pH 7.4. All solutions were filtered and degassed prior to a titration being carried out. The enthalpy change resulting from each injection of ligand was obtained through integration of the calorimetric signal. Data were analysed using Origin 7.0 (Microcal Software Inc., Northampton, Mass.). Heats of dilution were estimated using the final injections of each individual titration and subtracted before data fitting. Different ITC experimental formats were employed in order to obtain compound dissociation constants (Kd's) over a wide range of affinities. For weakly binding compounds a low c-value ITC method was used (Turnbull W. B. & Daranas A. H. J. Am. Chem. Soc. 2003 Dec. 3; 125(48):14859-66) in which the protein was present at 10-20 μM in the calorimetric cell and the compound concentration was 1-20 mM in the injection syringe. In this type of experiment the stoichiometry parameter (N) was locked at 1 for data fitting. For Kd's in the 20-0.004 μM range the experiment was configured such that the binding site concentration divided by the Kd (c-value) was between 5 and 1000. For the majority of these experiments the protein concentration in the calorimetric cell was in the range 4-100 μM and the ligand concentration in the injection syringe ranged from 50-1500 μM. In rare cases where compound solubility was limiting, the compound solution was placed in the calorimetric cell and titrated with protein from the injection syringe, maintaining a c-value between 5 and 1000.

The compounds of examples 3, 4, 21, 27, 29, 30, 34, 46, 47, 48, 49, 50, 51, 53, 54, 55, 56, 57, 59, 61, 64, 65, 66, 67, 68, 69, 70, 71, 72, 73, 75, 76, 77, 78, 79, 80, 81, 82 and 83 have been tested in the ITC assay and have all been found to have K_{d} values of less than 1 μM.

The compounds of examples 1, 33, 44, 45, 52, 60, 63 and 74 have K_{d} values of less than 10 μM.

The compounds of examples 58 and 62 have K_{d} values of less than 30 μM.

### Example 88

### Anti-Proliferative Activity

The anti-proliferative activities of compounds of the invention can be determined by measuring the ability of the compounds to inhibition of cell growth in a number of cell lines such as the human colon cancer cell line HCT116. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535 nM ex/590 nM em. In the case of the non-proliferating cell assay cells are maintained at confluence for 96 hour prior to the addition of inhibitor compounds for a further 72 hours. The number of viable cells is determined by Alamar Blue assay as before. Cell lines can be obtained from the ECACC (European Collection of cell Cultures).

### Pharmaceutical Formulations

### Example 89

### (i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

### (ii) Capsule Formulation

A capsule formulation is prepared by mixing 100 mg of a compound of the formula (I) with 100 mg lactose and filling the resulting mixture into standard opaque hard gelatin capsules.

### (iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

### v) Injectable Formulation III

A formulation for i.v. delivery by injection or infusion can be prepared by dissolving the compound of formula (I) (e.g. in a salt form) in water at 20 mg/ml. The vial is then sealed and sterilised by autoclaving.

### vi) Injectable Formulation IV

A formulation for i.v. delivery by injection or infusion can be prepared by dissolving the compound of formula (I) (e.g. in a salt form) in water containing a buffer (e.g. 0.2 M acetate pH 4.6) at 20 mg/ml. The vial is then sealed and sterilised by autoclaving.

### (vii) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

### viii) Lyophilised Formulation

Aliquots of formulated compound of formula (I) are put into 50 ml vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1.-43. (canceled)

44. A compound of the formula (IIa): 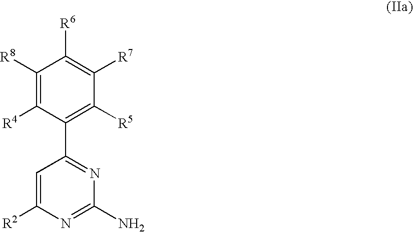
or a salt, tautomer, solvate or N-oxide thereof; wherein:
R² and R⁴ each are chlorine, and R⁵ to R⁸ each are hydrogen; or
R² is C₁₋₆ alkylthio; R⁴ and R⁵ each are methoxy; and R⁶, R⁷ and R⁸ each are hydrogen; or
R² is chlorine; R⁴ is chlorine; R⁶ is hydrogen or chlorine; R⁷ and R⁸ each are hydrogen; and R⁵ is a group R^{10cc} where R^{10cc} is selected from:
halogen;
CO₂R¹⁴ wherein R¹⁴ is hydrogen or C₁₋₆ alkyl;
C₁₋₆ alkyl optionally substituted by hydroxy, cyano or C₁₋₂ alkoxy;
C₁₋₆ alkoxy optionally substituted by hydroxy, cyano or C₁₋₂ alkoxy; or
a group [sol], CH₂[sol], C(O)[sol], OCH₂CH₂[sol] or OCH₂CH₂CH₂[sol] where [sol] is selected from: hydroxy, amino, C₁₋₄ alkylamino, di-C₁₋₄ alkylamino, 2-hydroxy-ethoxy, 2-methoxy-ethoxy, 2-amino-ethoxy, 2-amino-propoxy, 2-amino-2-methylpropoxy, 2-hydroxy-ethylamino, 2-hydroxy-propylamino, 2-hydroxy-2-methyl-propylamino, a group —O—(CH₂)ₚ(CR¹⁷R¹⁸)_{q}C(O)R¹⁶ 1-piperazino, 4-methyl-1-piperazino, 4-morpholino, 1-piperidino, 1-pyrrolidino, 1-imidazolyl, 4-methylsulphonyl-1-piperidino, 2-dimethylamino-1-hydroxyethyl, 1-dimethylamino-2-hydroxyethyl, 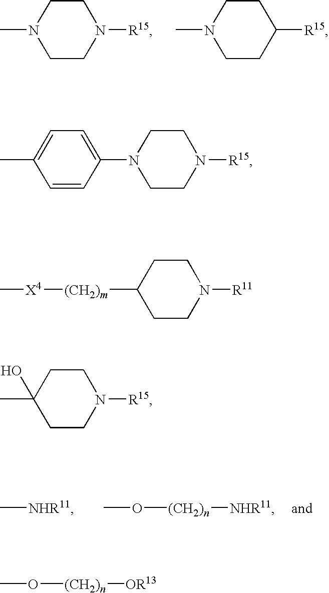
wherein p is 0, 1 or 2 and q is 0 or 1, provided that the sum of p and q is 1, 2 or 3:
R¹⁶ is OH; NH₂; NHMe; or C₁₋₄ alkoxy optionally substituted by a group R²¹ wherein R²¹ is selected from C₃₋₆ cycloalkyl, piperidine-4-yl, N—C₁₋₄ alkanoylpiperidin-4-yl, N—C₁₋₄alkoxycarbonyl-piperidin-4-yl, N—C₁₋₆ alkylpiperazine, piperazine, morpholine and tetrahydropyran;
R¹⁷ and R¹⁸ are each independently selected from hydrogen and methyl;
X⁴ is NH or O, m is 0 or 1, n is 1, 2 or 3;
R¹¹ is hydrogen, COR¹², C(O)OR¹² or R¹²;
R¹² is C₁₋₆ alkyl, C₃₋₆ cycloalkyl, aryl, aryl-C₁₋₆ alkyl or CH₂R¹⁵;
R¹⁵ is selected from hydrogen, C₁₋₆ alkyl, C₃₋₆ cycloalkyl, hydroxy-C₁₋₆ alkyl, piperidine, N—C₁₋₆ alkylpiperazine, piperazine, morpholine, COR¹³ and C(O)OR¹³; and
R¹³ is C₁₋₆ alkyl; or
R² is chlorine; R⁴ is chlorine; R⁵, R⁷ and R⁸ each are hydrogen; and R⁶ is a group R^{10cc}; or
R² is chlorine; R⁴ is chlorine or methoxy; R⁶ is chlorine; R⁵ and R⁸ each are hydrogen; and R⁷ is a group R^{10cc}; or
R² is chlorine; R⁴ is chlorine; R⁵, R⁶ and R⁷ each are hydrogen; and R⁸ is a group R^{10cc}.

45. A compound according to claim 44 wherein R² and R⁴ each are chlorine, and R⁵ to R⁸ each are hydrogen.

46. A compound according to claim 44 wherein R² is C₁₋₆ alkylthio; R⁴ and R⁵ each are methoxy; and R⁶, R⁷ and R⁸ each are hydrogen.

47. A compound according to claim 44 wherein R² is chlorine; R⁴ is chlorine; R⁶ is hydrogen or chlorine; R⁷ and R⁸ each are hydrogen; and R⁵ is a group R^{10cc}.

48. A compound according to claim 47 wherein R⁶ is hydrogen.

49. A compound according to claim 48 wherein R^{10cc} is:
fluorine;
chlorine;
C₁₋₆ alkyl optionally substituted by hydroxy, cyano or C₁₋₂ alkoxy;
C₁₋₆ alkoxy optionally substituted by hydroxy, cyano or C₁₋₂ alkoxy; or
a group [sol], CH₂[sol], OCH₂CH₂[sol] or OCH₂CH₂CH₂[sol] where [sol] is selected from: hydroxy, amino, C₁₋₄ alkylamino, di-C₁₋₄ alkylamino, 2-hydroxy-ethoxy, 2-methoxy-ethoxy, a group —O—(CH₂)ₚ(CR¹⁷R¹⁸)_{q}C(O)R¹⁶, 1-piperazino, 4-methyl-1-piperazino, 4-morpholino, 1-piperidino, 1-pyrrolidino, 1-imidazolyl and 4-methylsulphonyl-1-piperidino and NHR¹¹;
wherein p is 0, 1 or 2 and q is 0 or 1, provided that the sum of p and q is 1, 2 or 3;
R¹⁶ is OH, NH₂, NHMe or C₁₋₄ alkoxy;
R¹¹ is hydrogen, COR¹², C(O)OR¹² or R¹²;
R¹² is C₁₋₆ alkyl or CH₂R¹⁵;
R¹⁵ is selected from hydrogen, C₁₋₆ alkyl, hydroxy-C₁₋₆ alkyl, piperidine, N—C₁₋₄ alkylpiperazine, piperazine, morpholine, COR¹³ and C(O)OR¹³; and
R¹³ is C₁₋₄ alkyl.

50. A compound according to claim 47 wherein R⁶ is chlorine.

51. A compound according to claim 44 wherein R² is chlorine; R⁴ is chlorine; R⁵, R⁷ and R⁸ each are hydrogen; and R⁶ is a group R^{10cc}.

52. A compound according to claim 51 wherein R^{10cc} is:
chlorine;
C₁₋₆ alkyl optionally substituted by hydroxy, cyano or C₁₋₂ alkoxy;
C₁₋₆ alkoxy optionally substituted by hydroxy, cyano or C₁₋₂ alkoxy; or
a group [sol], CH₂[sol], C(O)[sol], OCH₂CH₂[sol] or OCH₂CH₂CH₂[sol] where [sol] is selected from: hydroxy, amino, C₁₋₄ alkylamino, di-C₁₋₄ alkylamino, 2-hydroxy-ethoxy, 2-methoxy-ethoxy, 2-amino-ethoxy, 2-amino-propoxy, 2-amino-2-methylpropoxy, 2-hydroxy-ethylamino, 2-hydroxy-propylamino, 2-hydroxy-2-methyl-propylamino, a group —O—(CH₂)ₚ(CR⁷R⁸)_{q}C(O)R¹⁶, 1-piperazino, 4-methyl-1-piperazino, 4-morpholino, 1-piperidino, 1-pyrrolidino, 1-imidazolyl, 4-methylsulphonyl-1-piperidino, 2-dimethylamino-1-hydroxyethyl, 1-dimethylamino-2-hydroxyethyl, NHR¹¹, and —O—(CH₂)ₙ—OR¹³
wherein p is 0, 1 or 2 and q is 0 or 1, provided that the sum of p and q is 1, 2 or 3:
R¹⁶ is OH; NH₂; NHMe; or C₁₋₄ alkoxy optionally substituted by a group R²¹ wherein R²¹ is selected from C₃₋₆ cycloalkyl, piperidine-4-yl, N—C₁₋₄ alkanoylpiperidin-4-yl, N—C₁₋₄alkoxycarbonyl-piperidin-4-yl, N—C₁₋₆ alkylpiperazine, piperazine, morpholine and tetrahydropyran;
R¹⁷ and R¹⁸ are each independently selected from hydrogen and methyl;
X⁴ is NH or O, m is 0 or 1, n is 1, 2 or 3;
R¹¹ is hydrogen, COR¹², C(O)OR¹² or R¹²;
R¹² is C₁₋₆ alkyl, C₃₋₆ cycloalkyl, or CH₂R⁵;
R¹⁵ is selected from hydrogen, C₁₋₆ alkyl, C₃₋₆ cycloalkyl, hydroxy-C₁₋₆ alkyl, piperidine, N—C₁₋₆ alkylpiperazine, piperazine, morpholine, COR¹³ and C(O)OR¹³; and
R¹³ is C₁₋₆ alkyl.

53. A compound according to claim 52 wherein R^{10cc} is hydroxy; chlorine; C₁₋₄ alkoxy; or a group —O—(CH₂)ₚ(CR¹⁷R¹⁸)_{q}C(O)R¹⁶.

54. A compound according to claim 53 wherein R^{10cc} is hydroxy, methoxy, ethoxy, isopropoxy, chlorine, or —O—(CH₂)ₚ(CR¹⁷R¹⁸)_{q}C(O)R¹⁶ where R¹⁶ is selected from NH₂; and C₁₋₂ alkoxy optionally substituted by a piperidine-4-yl or N—C₁₋₄ alkanoylpiperidin-4-yl group.

55. A compound according to claim 44 wherein R² is chlorine; R⁴ is chlorine or methoxy; R⁶ is chlorine; R⁵ and R⁸ each are hydrogen; and R⁷ is a group R^{10cc}.

56. A compound according to claim 55 wherein R⁴ is chlorine.

57. A compound according to claim 55 wherein R^{10cc} is chlorine;
C₁₋₆ alkyl optionally substituted by hydroxy, cyano or C₁₋₂ alkoxy;
C₁₋₆ alkoxy optionally substituted by hydroxy, cyano or C₁₋₂ alkoxy; or
a group [sol], CH₂[sol], C(O)[sol], OCH₂CH₂[sol] or OCH₂CH₂CH₂[sol] where [sol] is selected from: hydroxy, amino, C₁₋₄ alkylamino, di-C₁₋₄ alkylamino, 2-hydroxy-ethoxy, 2-methoxy-ethoxy, 2-amino-ethoxy, 2-amino-propoxy, 2-amino-2-methylpropoxy, 2-hydroxy-ethylamino, 2-hydroxy-propylamino, 2-hydroxy-2-methyl-propylamino, a group —O—(CH₂)ₚ(CR¹⁷R¹⁸)_{q}C(O)R¹⁶, 1-piperazino, 4-methyl-1-piperazino, 4-morpholino, 1-piperidino, 1-pyrrolidino, 1-imidazolyl, 4-methylsulphonyl-1-piperidino, 2-dimethylamino-1-hydroxyethyl, 1-dimethylamino-2-hydroxyethyl, NHR¹¹, and —O—(CH₂)ₙ—OR¹³
wherein p is 0, 1 or 2 and q is 0 or 1, provided that the sum of p and q is 1, 2 or 3:
R¹⁶ is OH; NH₂; NHMe; or C₁₋₄ alkoxy optionally substituted by a group R²¹ wherein R²¹ is selected from C₃₋₆ cycloalkyl, piperidine-4-yl, N—C₁₋₄ alkanoylpiperidin-4-yl, N—C₁₋₄alkoxycarbonyl-piperidin-4-yl, N—C₁₋₆ alkylpiperazine, piperazine, morpholine and tetrahydropyran;
R¹⁷ and R¹⁸ are each independently selected from hydrogen and methyl;
X⁴ is NH or O, m is 0 or 1, n is 1, 2 or 3;
R11 is hydrogen, COR¹², C(O)OR¹²or R¹²; R¹² is C₁₋₆ alkyl, C₃₋₆ cycloalkyl, or CH₂R¹⁵;
R¹⁵ is selected from hydrogen, C₁₋₆ alkyl, C₃₋₆ cycloalkyl, hydroxy-C₁₋₆ alkyl, piperidine, N—C₁₋₆ alkylpiperazine, piperazine, morpholine, COR¹³ and C(O)OR¹³; and
R¹³ is C₁₋₆ alkyl.

58. A compound according to claim 57 wherein R^{10cc} is hydroxy; chlorine; C₁₋₄ alkoxy; or a group —O—(CH₂)ₚ(CR¹⁷R¹⁸)_{q}C(O)R¹⁶.

59. A compound according to claim 44 wherein R² is chlorine; R⁴ is chlorine; R⁵, R⁶ and R⁷ each are hydrogen; and R⁸ is a group R^{10cc}.

60. A compound according to claim 59 wherein R⁸ is C₁₋₆ alkoxy or C₁₋₆ alkyl.

61. A compound according to claim 60 wherein R⁸ is methoxy or C₁₋₅ alkyl.

62. A compound of the formula (Ib): 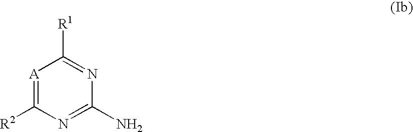
or salts, tautomers, solvates or N-oxides thereof; wherein:
A is N or a group CR³;
R¹ is a monocyclic or bicyclic carbocyclic or heterocyclic ring of 5 to 10 ring members of which up to two ring members may be heteroatoms selected from N, O and S and the remainder are carbon atoms, the carbocyclic or heterocyclic ring being optionally substituted by one or more substituent groups independently selected from R¹;
R² is selected from:
hydrogen halogen;
trifluoromethyl;
cyano;
amino;
mono- and di-C₁₋₄ hydrocarbylamino;
an acyclic C₁₋₁₀ hydrocarbyl group optionally substituted by one or more substituents R¹¹ and wherein one or more carbon atoms of the acyclic C¹⁻¹⁰ hydrocarbyl group may optionally be replaced by O, S, SO, SO₂, NR^{c}, X¹C(X²), C(X²)X¹ or X¹C(X²)X¹;
a group R^{d}-R^{c} wherein R^{d} is O, CO, X¹C(X²), C(X²)X¹, X¹C(X²)X¹, S, SO, SO₂, NR^{c}, SO₂NR^{c} or NR^{c}SO₂; and R^{e} is selected from hydrogen, carbocyclic and heterocyclic groups having from 3 to 12 ring members, and a C₁₋₁₀ hydrocarbyl group optionally substituted by one or more substituents R¹¹, and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; provided that R^{d}-R^{e} is not hydroxy;
R³ is selected from R² and monocyclic carbocyclic and heterocyclic groups having 3 to 7 ring members, wherein the monocyclic carbocyclic and heterocyclic groups are optionally substituted by one or more substituent groups independently selected from R¹⁰;
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₁₂ hydrocarbyl group (such as a C₁₋₁₀ hydrocarbyl group) optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; wherein the carbocyclic and heterocyclic groups of R¹⁰ may be unsubstituted or substituted by one or more further groups selected from R¹⁰, which further groups are not themselves further substituted;
R^{c} is selected from R^{b}, hydrogen and C₁₋₄ hydrocarbyl; and
X¹ is O, S or NR^{c} and X² is ═O, S or ═NR^{c}; and
R¹¹ is selected from hydroxy, oxo, halogen, cyano, nitro, carboxy, amino, mono- or di-C₁₋₄ hydrocarbylamino, carbocyclic and heterocyclic groups having from 3 to 12 ring members; wherein the carbocyclic and heterocyclic groups of R¹¹ may be unsubstituted or substituted by one or more further groups selected from R¹⁰; provided that the compound is other than:
(a-i) the compounds 4-chloro-6-phenyl-pyrimidin-2-ylamine, 4-(5-methyl-3-phenyl-isoxazol-4-yl)-pyrimidin-2-ylamine, 4-(2-thienyl)-2-pyrimidinamine, 2-amino-4-phenyl-6-methyl-1,3,5-triazine; 2-amino-4-(carboxymethyl)-6-phenyl-1,3,5-triazine; 2-amino-4-cyano-6-phenyl-1,3,5-triazine; 2-amino-4-phenylamino-6-phenyl-1,3,5-triazine; 2-amino-4-(carboxymethyl)-6-phenyl-1,3,5-triazine;
(a-ii) a compound wherein R² is an optionally substituted quinolinylamino group and R¹ is 4-fluorophenyl or 4-chlorophenyl;
(a-iii) a compound wherein A is CR³ wherein R³ is cyano or halogen;
(a-iv) a compound wherein R¹ is unsubstituted phenyl and R² is an acyclic hydrocarbyl group linked to an optionally substituted pyridyl group;
(a-v) a compound wherein R¹ is unsubstituted 3-pyridyl and R² is other than chlorine, amino or methyl;
(a-vi) a compound wherein R¹ is optionally substituted naphthyl or indolyl;
(a-vii) a compound wherein A is N, R² is amino and R¹ is a 3-phenoxyphenyl or 3-phenylsulphanylphenyl group;
(a-viii) a compound wherein A is CR³, R¹ is optionally substituted furanyl and R² contains a pyridylalkyl moiety;
(a-ix) a compound wherein A is CR³, R² is methyl and R¹ is an optionally substituted bicyclic group;
(a-x) a compound wherein A is CR³, R² is alkyl and R¹ is an indolyl group;
(a-xi) a compound wherein A is CR³, R² is methyl and R¹ is selected from 3-fluorophenyl, 3-chlorophenyl, 3-methoxyphenyl, 3-n-triphenyl, 3-chloro-4-fluorophenyl, 3,5-difluorophenyl, 4-amino-5-chloro-2-methoxphenyl and 3-trifluoromethylphenyl;
(a-xii) a compound wherein A is CR³, R² is isopropyl and R¹ is 4-amino-5-chloro-2-methoxphenyl;
(a-xiii) a compound wherein R¹ is optionally substituted dihydroquinolinyl;
(a-xiv) a compound wherein R¹ is a 2-hydroxy-5-aroyl-phenyl or 2-hydroxy-5-heteroaroyl-phenyl group;
(a-xv) a compound wherein A is CH, R² is hydrogen, and R¹ is other than a 3-pyridyl group substituted at the 2-position thereof with an aryl or heteroaryl group, and wherein the 4-, 5- and 6-positions of the 3-pyridyl group are optionally substituted;
(a-xvi) the compound irsogladine (6-(2,5-dichloro-phenyl)-[1,3,5]triazine-2,4-diamine;
(a-xvii) a compound wherein A is N and R² is a substituted or unsubstituted amino group, alkanoylamino group or a saturated 5- or 6-membered heterocyclic group;
(b-i) 4-chloro-6-(4-fluorophenyl)pyrimidinyl-2-amine and 4-chloro-6-(4-chlorophenyl)pyrimidinyl-2-amine;
(b-ii) 2,4-diamino-4-phenyltriazine;
(b-iii) 2-benzoylamino-6-amino-4-phenyltriazine;
(b-iv) 2-amino-4-n-butylamino-6-phenyl-[1,3,5]-triazine;
(b-v) 6-{4-[ethyl-(2-methoxy-ethyl)-amino]-2-methyl-phenyl}-[1,3,5]triazine-2,4-diamine;
(b-vi) a compound wherein A is N and R² is C₁₋₄ alkoxy or C₁₋₂ alkoxy-C₁₋₂ alkoxy;
(b-vii) 4-methylsulphanyl-6-(4-phenoxy-phenyl)-[1,3,5]triazin-2-ylamine;
(b-viii) a compound wherein R² is methanesulphinyl;
(b-ix) a compound wherein R¹ is furanyl;
(b-x) a compound wherein R¹ is a phenyl group bearing a hydroxy or benzyloxy substituent at the 2-position thereof and an optionally substituted aryl, heteroaryl, amide, ester, aroyl or heteroaroyl group at the 5-position thereof;
(b-xi) a compound wherein R¹ is a phenyl group bearing a hydroxy or benzyloxy substituent at the 4-position thereof and an optionally substituted aryl or heteroaryl group at the 3-position thereof;
(b-xii) a compound wherein R¹ is a 2,5-disubstituted phenyl group bearing an alkyl, halogen or alkoxy substituent at the 2-position thereof and a halogen or alkoxy substituent at the 5-position thereof;
(b-xiii) a compound wherein R² is chlorine and R¹ is a dimethoxyphenyl or trimethoxyphenyl group; and
(b-xiv) the compounds:
4-chloro-6-(2,3,5-trichlorophenyl)-2-pyrimidinamine;
4-chloro-6-(2,4-difluorophenyl)-2-pyrimidinamine;
4-(4-amino-5-chloro-2-methoxyphenyl)-6-methyl-2-pyrimidinamine;
4-chloro-6-(2-methylphenyl)-2-pyrimidinamine;
4-chloro-6-(2-methoxyphenyl)-2-pyrimidinamine;
4-(2-chlorophenyl)-6-ethoxy-2-pyrimidinamine.

63. A compound according to claim 44 in the form of a salt, solvate or N-oxide.

64. A method for alleviating or reducing the incidence of a disease state or condition mediated by Hsp90, which method comprises administering to a subject in need thereof a compound having the formula (I): 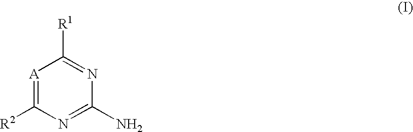
or salts, tautomers, solvates or N-oxides thereof; wherein:
A is N or a group CR³;
R¹ is a monocyclic or bicyclic carbocyclic or heterocyclic ring of 5 to 10 ring members of which up to two ring members may be heteroatoms selected from N, O and S and the remainder are carbon atoms, the carbocyclic or heterocyclic ring being optionally substituted by one or more substituent groups independently selected from R¹⁰;
R² is selected from:
hydrogen
halogen;
trifluoromethyl;
cyano;
amino;
mono- and di-C₁₋₄ hydrocarbylamino;
an acyclic C₁₋₁₀ hydrocarbyl group optionally substituted by one or more substituents independently selected from R¹¹ and wherein one or more carbon atoms of the acyclic C₁₋₁₀ hydrocarbyl group may optionally be replaced by O, S, SO, SO₂, NR^{c}, X¹C(X¹), C(X¹)X¹ or X¹C(X²)X¹;
a group R^{d}-R^{e} wherein R^{d} is O, CO, X¹C(X²), C(X²)X¹, X¹C(X²)X¹, S, SO, SO₂, NR^{c}, SO₂NR^{c} or NR^{c}SO₂; and R^{e} is selected from hydrogen, carbocyclic and heterocyclic groups having from 3 to 12 ring members, and a C₁₋₁₀ hydrocarbyl group optionally substituted by one or more substituents R¹¹, and wherein one or more carbon atoms of the C¹⁻¹⁰ hydrocarbyl group may optionally be replaced by O, S, SO, SO₂, NR^{c}, X¹C(X²), C(X²)X¹ or X¹C(X²)X¹; provided that R^{d}-R^{e} is not hydroxy;
R³ is selected from R² and monocyclic carbocyclic and heterocyclic groups having 3 to 7 ring members, wherein the monocyclic carbocyclic and heterocyclic groups are optionally substituted by one or more substituent groups independently selected from R¹⁰;
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₁₂ hydrocarbyl group (such as a C₁₋₁₀ hydrocarbyl group) optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; wherein the carbocyclic and heterocyclic groups of R¹⁰ may be unsubstituted or substituted by one or more further groups selected from R¹⁰, which further groups are not themselves further substituted;
R^{c} is selected from R^{b}, hydrogen and C₁₋₄ hydrocarbyl; and
X¹ is O, S or NR^{c} and X² is ═O, —S or ═NR^{c}; and
R¹¹ is selected from hydroxy, oxo, halogen, cyano, nitro, carboxy, amino, mono- or di-C₁₋₄ hydrocarbylamino, carbocyclic and heterocyclic groups having from 3 to 12 ring members; wherein the carbocyclic and heterocyclic groups of R¹¹ may be unsubstituted or substituted by one or more further groups selected from R¹⁰.

65. A method for treating (or alleviating or reducing the incidence of) a disease or condition comprising or arising from abnormal cell growth in a mammal, which method comprises administering to the mammal a compound of the formula (I): 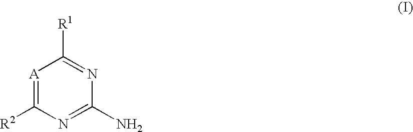
or salts, tautomers, solvates or N-oxides thereof; wherein:
A is N or a group CR³;
R¹ is a monocyclic or bicyclic carbocyclic or heterocyclic ring of 5 to 10 ring members of which up to two ring members may be heteroatoms selected from N, O and S and the remainder are carbon atoms, the carbocyclic or heterocyclic ring being optionally substituted by one or more substituent groups independently selected from R¹⁰;
R² is selected from:
hydrogen
halogen;
trifluoromethyl;
cyano;
amino;
mono- and di-C₁₋₄ hydrocarbylamino;
an acyclic C₁₋₁₀ hydrocarbyl group optionally substituted by one or more substituents independently selected from R¹¹ and wherein one or more carbon atoms of the acyclic C₁₋₁₀ hydrocarbyl group may optionally be replaced by O, S, SO, SO₂, NR^{c}, X¹C(X²), C(X²)X¹ or X¹C(X²)X¹;
a group R^{d}-R^{e} wherein R^{d} is O, CO, X¹C(X²), C(X²)X¹, X¹C(X²)X¹, S, SO, SO₂, NR^{c}, SO₂NR^{c} or NR^{c}SO₂; and R^{e} is selected from hydrogen, carbocyclic and heterocyclic groups having from 3 to 12 ring members, and a C₁₋₁₀ hydrocarbyl group optionally substituted by one or more substituents R¹¹, and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; provided that R^{d}-R^{e} is not hydroxy;
R³ is selected from R² and monocyclic carbocyclic and heterocyclic groups having 3 to 7 ring members, wherein the monocyclic carbocyclic and heterocyclic groups are optionally substituted by one or more substituent groups independently selected from R¹⁰;
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₁₂ hydrocarbyl group (such as a C₁₋₁₀ hydrocarbyl group) optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; wherein the carbocyclic and heterocyclic groups of R¹⁰ may be unsubstituted or substituted by one or more further groups selected from R¹⁰, which further groups are not themselves further substituted;
R^{c} is selected from R^{b}, hydrogen and C₁₋₄ hydrocarbyl; and
X¹ is O, S or NR^{c} and X² is ═O, ═S or ═NR^{c}; and
R¹¹ is selected from hydroxy, oxo, halogen, cyano, nitro, carboxy, amino, mono- or di-C₁₋₄ hydrocarbylamino, carbocyclic and heterocyclic groups having from 3 to 12 ring members; wherein the carbocyclic and heterocyclic groups of R¹¹ may be unsubstituted or substituted by one or more further groups selected from R¹⁰.

66. A method for the diagnosis and treatment of a disease state or condition mediated by Hsp90, which method comprises (i) screening a patient to determine whether a disease or condition from which the patient is or may be suffering is one which would be susceptible to treatment with a compound having activity against Hsp90; and (ii) where it is indicated that the disease or condition from which the patient is thus susceptible, thereafter administering to the patient a compound of the formula (I): 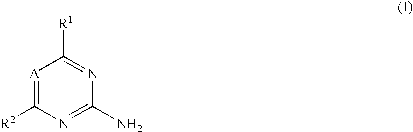
or salts, tautomers, solvates or N-oxides thereof; wherein:
A is N or a group CR³;
R¹ is a monocyclic or bicyclic carbocyclic or heterocyclic ring of 5 to 10 ring members of which up to two ring members may be heteroatoms selected from N, O and S and the remainder are carbon atoms, the carbocyclic or heterocyclic ring being optionally substituted by one or more substituent groups independently selected from R¹⁰;
R² is selected from:
hydrogen
halogen;
trifluoromethyl;
cyano;
amino;
mono- and di-C₁₋₄ hydrocarbylamino;
an acyclic C₁₋₁₀ hydrocarbyl group optionally substituted by one or more substituents independently selected from R¹¹ and wherein one or more carbon atoms of the acyclic C¹⁻¹⁰ hydrocarbyl group may optionally be replaced by O, S, SO, SO₂, NR^{c}, X¹C(X²), C(X²)X¹ or X¹C(X²)X¹;
a group R^{d}-R^{e} wherein R^{d} is O, CO, X¹C(X²), C(X²)X¹, X¹C(X²)X¹, S, SO, SO₂, NR^{c}, SO₂NR^{c} or NR^{c}SO₂; and R^{e} is selected from hydrogen, carbocyclic and heterocyclic groups having from 3 to 12 ring members, and a C₁₋₁₀ hydrocarbyl group optionally substituted by one or more substituents R¹¹, and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; provided that R^{d}-R^{e} is not hydroxy;
R³ is selected from R² and monocyclic carbocyclic and heterocyclic groups having 3 to 7 ring members, wherein the monocyclic carbocyclic and heterocyclic groups are optionally substituted by one or more substituent groups independently selected from R¹⁰;
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₁₂ hydrocarbyl group (such as a C₁₋₁₀ hydrocarbyl group) optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; wherein the carbocyclic and heterocyclic groups of R¹⁰ may be unsubstituted or substituted by one or more further groups selected from R¹⁰, which further groups are not themselves further substituted;
R^{c} is selected from R^{b}, hydrogen and C₁₋₄ hydrocarbyl; and
X¹ is O, S or NR^{c} and X² is ═O , ═S or ═NR^{c}; and
R¹¹ is selected from hydroxy, oxo, halogen, cyano, nitro, carboxy, amino, mono- or di-C₁₋₄ hydrocarbylamino, carbocyclic and heterocyclic groups having from 3 to 12 ring members; wherein the carbocyclic and heterocyclic groups of R¹¹ may be unsubstituted or substituted by one or more further groups selected from R¹⁰.

67. A pharmaceutical composition comprising a compound of the formula (Ia): 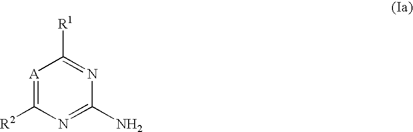
or salts, tautomers, solvates or N-oxides thereof; wherein:
A is N or a group CR³;
R¹ is a monocyclic or bicyclic carbocyclic or heterocyclic ring of 5 to 10 ring members of which up to two ring members may be heteroatoms selected from N, O and S and the remainder are carbon atoms, the carbocyclic or heterocyclic ring being optionally substituted by one or more substituent groups independently selected from R¹⁰;
R² is selected from:
hydrogen
halogen;
trifluoromethyl;
cyano;
amino;
mono- and di-C₁₋₄ hydrocarbylamino;
an acyclic C₁₋₁₀ hydrocarbyl group optionally substituted by one or more substituents R¹¹ and wherein one or more carbon atoms of the acyclic C₁₋₁₀ hydrocarbyl group may optionally be replaced by O, S, SO, SO₂, NR^{c}, X¹C(X²), C(X²)X or X¹C(X²)X¹;
a group R^{d}-R^{e} wherein R^{d} is O, CO, X¹C(X²), C(X²)X¹, X¹C(X²)X¹, S, SO, SO₂, NR^{c}, SO₂NR^{c} or NR^{c}SO₂; and R^{e} is selected from hydrogen, carbocyclic and heterocyclic groups having from 3 to 12 ring members, and a C₁₋₁₀ hydrocarbyl group optionally substituted by one or more substituents R¹¹, and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; provided that R^{d}-R^{e} is not hydroxy;
R³ is selected from R² and monocyclic carbocyclic and heterocyclic groups having 3 to 7 ring members, wherein the monocyclic carbocyclic and heterocyclic groups are optionally substituted by one or more substituent groups independently selected from R¹⁰;
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₁₂ hydrocarbyl group (such as a C₁₋₁₀ hydrocarbyl group) optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₁₀ hydrocarbyl group may optionally be replaced by O, S, SO, SO₂, NR^{c}, X¹C(X²), C(X²)X¹ or X¹C(X²)X¹; wherein the carbocyclic and heterocyclic groups of R¹⁰ may be unsubstituted or substituted by one or more further groups selected from R¹⁰, which further groups are not themselves further substituted;
R^{c} is selected from R^{b}, hydrogen and C₁₋₄ hydrocarbyl; and
X¹ is O, S or NR^{c} and X² is ═O, ═S or ═NR^{c}; and
R¹¹ is selected from hydroxy, oxo, halogen, cyano, nitro, carboxy, amino, mono- or di-C₁₋₄ hydrocarbylamino, carbocyclic and heterocyclic groups having from 3 to 12 ring members; wherein the carbocyclic and heterocyclic groups of R¹¹ may be unsubstituted or substituted by one or more further groups selected from R¹⁰; provided that the compound is other than:
(a-i) the compounds 4-chloro-6-phenyl-pyrimidin-2-ylamine, 4-(5-methyl-3-phenyl-isoxazol-4-yl)-pyrimidin-2-ylamine, 4-(2-thienyl)-2-pyrimidinamine, 2-amino-4-phenyl-6-methyl-1,3,5-triazine; 2-amino-4-(carboxymethyl)-6-phenyl-1,3,5-triazine; 2-amino-4-cyano-6-phenyl-1,3,5-triazine; 2-amino-4-phenylamino-6-phenyl-1,3,5-triazine; 2-amino-4-(carboxymethyl)-6-phenyl-1,3,5-triazine;
(a-ii) a compound wherein R² is an optionally substituted quinolinylamino group and R¹ is 4-fluorophenyl or 4-chlorophenyl;
(a-iii) a compound wherein A is CR³ wherein R³ is cyano or halogen;
(a-iv) a compound wherein R¹ is unsubstituted phenyl and R² is an acyclic hydrocarbyl group linked to an optionally substituted pyridyl group;
(a-v) a compound wherein R¹ is unsubstituted 3-pyridyl and R² is other than chlorine, amino or methyl;
(a-vi) a compound wherein R¹ is optionally substituted naphthyl, indolyl, quinolinyl or isoquinolinyl;
(a-vii) a compound wherein A is N, R² is amino and R¹ is a 3-phenoxyphenyl or 3-phenylsulphanylphenyl group;
(a-viii) a compound wherein A is CR³, R¹ is optionally substituted furanyl and R² contains a pyridylalkyl moiety;
(a-ix) a compound wherein A is CR³, R² is an optionally substituted alkyl group and R¹ is an optionally substituted bicyclic group;
(a-x) a compound wherein A is CR³, R² is alkyl and R¹ is an indolyl group;
(a-xi) a compound wherein A is CR³, R² is methyl and R¹ is selected from 5-chlorothiophen-2-yl, 3-fluorophenyl, 3-chlorophenyl, 3-methoxyphenyl, 3-n-triphenyl, 3-chloro-4-fluorophenyl, 3,5-difluorophenyl, 3,5-dichlorophenyl, 4-amino-5-chloro-2-methoxphenyl and 3-trifluoromethylphenyl;
(a-xii) a compound wherein A is CR³, R² is isopropyl and R¹ is 4-amino-5-chloro-2-methoxphenyl;
(a-xiii) a compound wherein R¹ is optionally substituted quinolinyl or dihydroquinolinyl;
(a-xiv) a compound wherein R¹ is a 2-hydroxy-5-aroyl-phenyl or 2-hydroxy-5-heteroaroyl-phenyl group;
(a-xv) a compound wherein A is CH, R² is hydrogen, and R¹ is other than a 3-pyridyl group substituted at the 2-position thereof with an aryl or heteroaryl group, and wherein the 4-, 5- and 6-positions of the 3-pyridyl group are optionally substituted;
(a-xvi) the compound irsogladine (6-(2,5-dichloro-phenyl)-[1,3,5]triazine-2,4-diamine; and
(a-xvii) a compound wherein A is N and R² is a substituted or unsubstituted amino group, alkanoylamino group or a saturated 5- or 6-membered heterocyclic group;
and a pharmaceutically acceptable carrier.

68. A compound according to claim 44 in the form of a pharmaceutical composition additionally comprising a pharmaceutically acceptable carrier.

69. A method of preparing a compound as defined in claim 44, which process comprises
the reaction of a compound of the formula (X): 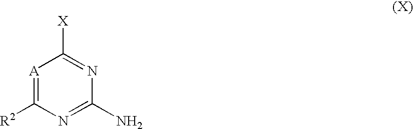
or an N-protected derivative thereof, with a compound of the formula R¹—Y; wherein R¹ is the moiety: 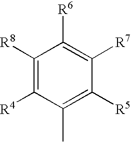
or is as defined in any one of the preceding claims, and one of X and Y is selected from chlorine, bromine, iodine and trifluoro-methanesulphonate; and the other of X and Y is a boronate residue such as a boronic acid group or a boronate ester, under Suzuki coupling conditions, and thereafter optionally removing any protecting groups present and optionally converting one compound of the formula (I) into another compound of the formula (I); or
the reaction of a compound of the formula (XI): 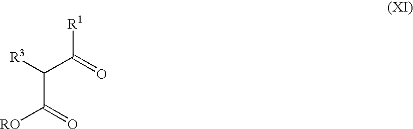
wherein OR is an alkoxy group such as methoxy; with guanidine or a salt thereof to give a compound of the formula (XII): 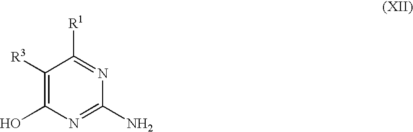
and thereafter replacing the hydroxy group in formula (XII) with a chlorine atom by reaction with a chlorinating agent to give a compound of the formula (XIII): 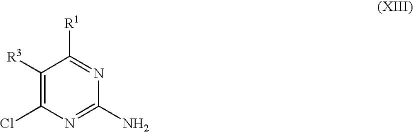
or
(iii) the reaction of an enamine compound of formula (XV): 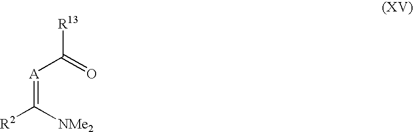
with guanidine or a salt thereof (e.g. the hydrochloride) to give a compound of the formula (I) in which R² is as defined in claim 54 provided that R² is a group that does not interefere with the reaction of the enamine compound; or
(iv) the conversion of one compound of the formula (I) into another compound of the formula (I).

70. A method for treating (or alleviating or reducing the incidence of) a disease or condition comprising or arising from abnormal cell growth in a mammal, which method comprises administering to the mammal a compound of the formula (IIa) as defined in claim 44, or a salt, tautomer, solvate or N-oxide thereof.

71. A method for treating (or alleviating or reducing the incidence of) a disease or condition comprising or arising from abnormal cell growth in a mammal, which method comprises administering to the mammal a compound of the formula (Ib) as defined in claim 62, or a salt, tautomer, solvate or N-oxide thereof

72. A compound according to claim 62 in the form of a pharmaceutical composition additionally comprising a pharmaceutically acceptable carrier.

73. A method for treating (or alleviating or reducing the incidence of) a disease or condition comprising or arising from abnormal cell growth in a mammal, which method comprises administering to the mammal a compound of the formula (Ia) as defined in claim 67, or a salt, tautomer, solvate or N-oxide thereof.
